SEQUENCE LISTING (164 sequences)

SEQ ID NO: 1 - protein, 112 aa, Simian immunodeficiency virus
MSDPRERIPPGNSGEETIEEAFEWLNRTVEGINRAAVNHLPRELIFQVWQRSWEYWHDEMGMSESYTKYRYLCLIQKALFMHCKKGCRCLGEGHGAGGWRTGPPPPPPPGLA

SEQ ID NO: 2 - protein, 113 aa, Human immunodeficiency virus 2
MTDPRERVPPGNSGEETIGEAFEWLERTIEALNREAVNHLPRELIFQVWQRSWRYWHDEQGMSASYTKYRYLCLMQKAIFTHFKRGCTCWGEDMGREGLEDQGPPPPPPPGLV

SEQ ID NO: 3 - DNA, 339 bp, Simian immunodeficiency virus
  source 1..339: mol_type = genomic DNA
atgtcagatcccagggagagaatcccacctggaaacagtggagaagagacaataggagaggccttcgaatggctaaacagaacagtagaggagataaacagagaggcagtaaaccacctaccaagggagctgattttccaggtttggcaaaggtcttgggaatactggcatgatgaacaagggatgtcacaaagctatgtaaaatacagatacttgtgtttaatgcaaaaggctttatttatgcattgcaagaaaggctgtagatgtctaggggaaggacacggggcaggaggatggagaccaggacctcctcctcctccccctccaggactagcatga

SEQ ID NO: 4 - DNA, 342 bp, Human immunodeficiency virus 2
  source 1..342: mol_type = genomic DNA
atgacagaccccagagaaagggtaccgccaggaaacagtggagaagagaccattggagaggccttcgagtggctagagaggaccatagaagccttaaacagggaggcagtgaaccatctgccccgagagctcattttccaggtgtggcaaaggtcctggagatattggcatgatgaacaagggatgtcagcaagctacacaaagtatagatatttgtgcctaatgcaaaaagctatatttacacatttcaagagagggtgcacttgctggggggaggacatgggccgggaaggattggaagaccaaggacctccccctcctccccctccaggtctagtctaa

SEQ ID NO: 5 - RNA, 119 nt, synthetic construct
  source 1..119: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gttcctcttctgtagcagctgttttagagctagaaatagcaagttaaaataaggctagtccgttatcaacttgaaaaagtggcaccgagtcggtgctagcatcagagctgctacagaagagcgcggttctatctagttacgcgttaaaccaactagaattt

SEQ ID NO: 7 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gttcctcttctgtagcagctgttttagagctagaaatagcaagttaaaataaggctagtccgttatcaacttgaaaaagtggcaccgagtcggtgctagcatcagggctgctacagaagagcgcggttctatctagttacgcgttaaaccaactagaattt

SEQ ID NO: 8 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gttcctcttctgtagcagctgttttagagctagaaatagcaagttaaaataaggctagtccgttatcaacttgaaaaagtggcaccgagtcggtgctagcattagggctgctacagaagagcgcggttctatctagttacgcgttaaaccaactagaattt

SEQ ID NO: 9 - RNA, 171 nt, synthetic construct
  source 1..171: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 168: mod_base = OTHER; note = Nucleotide with phosphorothioate linkage
  modified_base 169: mod_base = OTHER; note = 2'-O-methyl modified nucleotide with phosphorothioate linkage
  modified_base 170: mod_base = OTHER; note = 2'-O-methyl modified nucleotide with phosphorothioate linkage
  modified_base 171: mod_base = OTHER; note = 2'-O-methyl modified nucleotide
catggtgcacctgactcctggttttagagctagaaatagcaagttaaaataaggctagtccgttatcaacttgaaaaagtggcaccgagtcggtgcagacttctcttcaggagtcaggtgcacagaataaacgcggttctatctagttacgcgttaaaccaactagaattt

SEQ ID NO: 10 - RNA, 188 nt, synthetic construct
  source 1..188: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 185: mod_base = OTHER; note = Nucleotide with phosphorothioate linkage
  modified_base 186: mod_base = OTHER; note = 2'-O-methyl modified nucleotide with phosphorothioate linkage
  modified_base 187: mod_base = OTHER; note = 2'-O-methyl modified nucleotide with phosphorothioate linkage
  modified_base 188: mod_base = OTHER; note = 2'-O-methyl modified nucleotide
gcagctcaggttctgggagagttttagagctagaaatagcaagttaaaataaggctagtccgttatcaacttgaaaaagtggcaccgagtcggtgctaccgtacaccactgagaccgcggtggttgaccagacaaacctcccagaacccgcggttctatctagttacgcgttaaaccaactagaattt

SEQ ID NO: 11 - RNA, 191 nt, synthetic construct
  source 1..191: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 188: mod_base = OTHER; note = Nucleotide with phosphorothioate linkage
  modified_base 189: mod_base = OTHER; note = 2'-O-methyl modified nucleotide with phosphorothioate linkage
  modified_base 190: mod_base = OTHER; note = 2'-O-methyl modified nucleotide with phosphorothioate linkage
  modified_base 191: mod_base = OTHER; note = 2'-O-methyl modified nucleotide
gatggagccagagaggatccgttttagagctagaaatagcaagttaaaataaggctagtccgttatcaacttgaaaaagtggcaccgagtcggtgcgtctggtcaaccaccgcggtctcagtggtgtacggtacaaaccttcctctctggccgcggttctatctagttacgcgttaaaccaactagaattt

SEQ ID NO: 12 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gagtagcgcgagcacagctagttttagagctagaaatagcaagttaaaataaggctagtccgttatcaacttgaaaaagtggcaccgagtcggtgctccgtggcctgagctgtgctcgcgccgcggttctatctagttacgcgttaaaccaactagaattt

SEQ ID NO: 13 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gagtagcgcgagcacagctagttttagagctagaaatagcaagttaaaataaggctagtccgttatcaacttgaaaaagtggcaccgagtcggtgctccgtggcgtgagctgtgctcgcgccgcggttctatctagttacgcgttaaaccaactagaattt

SEQ ID NO: 14 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gagtagcgcgagcacagctagttttagagctagaaatagcaagttaaaataaggctagtccgttatcaacttgaaaaagtggcaccgagtcggtgctccgtggggtgagctgtgctcgcgccgcggttctatctagttacgcgttaaaccaactagaattt

SEQ ID NO: 15 - RNA, 161 nt, synthetic construct
  source 1..161: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 158: mod_base = OTHER; note = Nucleotide with phosphorothioate linkage
  modified_base 159: mod_base = OTHER; note = 2'-O-methyl modified nucleotide with phosphorothioate linkage
  modified_base 160: mod_base = OTHER; note = 2'-O-methyl modified nucleotide with phosphorothioate linkage
  modified_base 161: mod_base = OTHER; note = 2'-O-methyl modified nucleotide
gagtagcgcgagcacagctagttttagagctagaaatagcaagttaaaataaggctagtccgttatcaacttgaaaaagtggcaccgagtcggtgctccgtggggtaggctgtgctcgcgccgcggttctatctagttacgcgttaaaccaactagaattt

SEQ ID NO: 16 - RNA, 150 nt, synthetic construct
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 146: mod_base = OTHER; note = Nucleotide with phosphorothioate linkage
  modified_base 147: mod_base = OTHER; note = 2'-O-methyl modified nucleotide with phosphorothioate linkage
  modified_base 148: mod_base = OTHER; note = 2'-O-methyl modified nucleotide with phosphorothioate linkage
  modified_base 149: mod_base = OTHER; note = 2'-O-methyl modified nucleotide
gctgtgtttgcgtctctcccgttttagagctagaaatagcaagttaaaataaggctagtccgttatcaacttgaaaaagtggcaccgagtcggtgcatcctgacgacggagaccgccgtcgtcgacaagccagagacgcaaacatttttt

SEQ ID NO: 17 - RNA, 149 nt, synthetic construct
  source 1..149: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 145: mod_base = OTHER; note = Nucleotide with phosphorothioate linkage
  modified_base 146: mod_base = OTHER; note = 2'-O-methyl modified nucleotide with phosphorothioate linkage
  modified_base 147: mod_base = OTHER; note = 2'-O-methyl modified nucleotide with phosphorothioate linkage
  modified_base 148: mod_base = OTHER; note = 2'-O-methyl modified nucleotide
gtatggaaaatgagagctgcgttttagagctagaaatagcaagttaaaataaggctagtccgttatcaacttgaaaaagtggcaccgagtcggtgcttgtcgacgacggcggtctccgtcgtcaggatcatgctctcattttctttttt

SEQ ID NO: 18 - RNA, 131 nt, synthetic construct
  source 1..131: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 127: mod_base = OTHER; note = Nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide
gtcatcttagtcattacctggttttagagctagaaatagcaagttaaaataaggctagtccgttatcaacttgaaaaagtggcaccgagtcggtgcaacgaacaccagccgtaatgactaagatgtttttt

SEQ ID NO: 19 - RNA, 130 nt, synthetic construct
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
gcattgagatagtgtggggagttttagagctagaaatagcaagttaaaataaggctagtccgttatcaacttgaaaaagtggcaccgagtcggtgcttgggggcccattctccacactatctcatttttt

SEQ ID NO: 20 - RNA, 130 nt, synthetic construct
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
gcattgagatagtgtggggagttttagagctagaaatagcaagttaaaataaggctagtccgttatcaacttgaaaaagtggcaccgagtcggtgcttgggggcccgttctccacactatctcatttttt

SEQ ID NO: 21 - RNA, 130 nt, synthetic construct
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
gcattgagatagtgtggggagttttagagctagaaatagcaagttaaaataaggctagtccgttatcaacttgaaaaagtggcaccgagtcggtgcttgggggccggttgtccacactatctcatttttt

SEQ ID NO: 22 - RNA, 130 nt, synthetic construct
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
ctaacagttgcttttatcacgttttagagctagaaatagcaagttaaaataaggctagtccgttatcaacttgaaaaagtggcaccgagtcggtgcttcctggagcctgccataaaagcaactgtttttt

SEQ ID NO: 23 - RNA, 130 nt, synthetic construct
  source 1..130: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
ctaacagttgcttttatcacgttttagagctagaaatagcaagttaaaataaggctagtccgttatcaacttgaaaaagtggcaccgagtcggtgcttcctggagggtgccataaaagcaactgtttttt

SEQ ID NO: 24 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gagttctgtaattcagcatagttttagagctagaaatagcaagttaaaataaggctagtccgttatcaacttgaaaaagtggcaccgagtcggtgctttt

SEQ ID NO: 25 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
ccttgataccaacctgcccagttttagagctagaaatagcaagttaaaataaggctagtccgttatcaacttgaaaaagtggcaccgagtcggtgctttt

SEQ ID NO: 26 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gtaacggcagacttctcttcgttttagagctagaaatagcaagttaaaataaggctagtccgttatcaacttgaaaaagtggcaccgagtcggtgctttt

SEQ ID NO: 27 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
agtggaggcgtcgcgctggcgttttagagctagaaatagcaagttaaaataaggctagtccgttatcaacttgaaaaagtggcaccgagtcggtgctttt

SEQ ID NO: 28 - RNA, 100 nt, synthetic construct
  source 1..100: mol_type = other RNA
  modified_base 1: mod_base = OTHER; note = 2'-O-methyl modified thymine with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 8: mod_base = OTHER; note = Thymine
  modified_base 9: mod_base = OTHER; note = Thymine
  modified_base 20: mod_base = OTHER; note = Thymine
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
tcaaccattaagcaaaacatgttttagagctagaaatagcaagttaaaataaggctagtccgttatcaacttgaaaaagtggcaccgagtcggtgctttt

SEQ ID NO: 29 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
ggaatgactgaatcggaacagttttagagctagaaatagcaagttaaaataaggctagtccgttatcaacttgaaaaagtggcaccgagtcggtgctttt

SEQ ID NO: 30 - DNA, 24 bp, synthetic construct
  source 1..24: mol_type = other DNA
tctgggtgttatgagttccttggg

SEQ ID NO: 31 - DNA, 22 bp, synthetic construct
  source 1..22: mol_type = other DNA
aacatgaatcgctgtgctctgc

SEQ ID NO: 32 - DNA, 23 bp, synthetic construct
  source 1..23: mol_type = other DNA
ctgagggtttgaagtccaactcc

SEQ ID NO: 33 - DNA, 26 bp, synthetic construct
  source 1..26: mol_type = other DNA
cctgttacttatccccttcctatgac

SEQ ID NO: 34 - DNA, 24 bp, synthetic construct
  source 1..24: mol_type = other DNA
cctccagcctgaagtcctagaatg

SEQ ID NO: 35 - DNA, 22 bp, synthetic construct
  source 1..22: mol_type = other DNA
gcgaaagagcggaagagaaacc

SEQ ID NO: 36 - DNA, 23 bp, synthetic construct
  source 1..23: mol_type = other DNA
gccaaaagtttccatcaagcctc

SEQ ID NO: 37 - DNA, 24 bp, synthetic construct
  source 1..24: mol_type = other DNA
cagtcttccttggtgccttatcag

SEQ ID NO: 38 - DNA, 45 bp, synthetic construct
  source 1..45: mol_type = other DNA
ctacacgacgctcttccgatctgggggttctcaatgttactgtgg

SEQ ID NO: 39 - DNA, 44 bp, synthetic construct
  source 1..44: mol_type = other DNA
agacgtgtgctcttccgatctgaaggcaacagctgttcataacc

SEQ ID NO: 40 - DNA, 43 bp, synthetic construct
  source 1..43: mol_type = other DNA
ctacacgacgctcttccgatctagggctgggcataaaagtcag

SEQ ID NO: 41 - DNA, 43 bp, synthetic construct
  source 1..43: mol_type = other DNA
agacgtgtgctcttccgatcttgcccagtttctattggtctcc

SEQ ID NO: 42 - DNA, 45 bp, synthetic construct
  source 1..45: mol_type = other DNA
ctacacgacgctcttccgatctcatttcctggagccatctctctc

SEQ ID NO: 43 - DNA, 44 bp, synthetic construct
  source 1..44: mol_type = other DNA
agacgtgtgctcttccgatcttcttgggaagtgtaaggaagctg

SEQ ID NO: 44 - DNA, 45 bp, synthetic construct
  source 1..45: mol_type = other DNA
ctacacgacgctcttccgatctctttaaaagccaggacggtcacc

SEQ ID NO: 45 - DNA, 44 bp, synthetic construct
  source 1..44: mol_type = other DNA
agacgtgtgctcttccgatctccgagtagcagatgaccatgaca

SEQ ID NO: 46 - DNA, 44 bp, synthetic construct
  source 1..44: mol_type = other DNA
ctacacgacgctcttccgatctcgcgtttaatataagtggaggc

SEQ ID NO: 47 - DNA, 43 bp, synthetic construct
  source 1..43: mol_type = other DNA
agacgtgtgctcttccgatctggagaacttggagaagggaagt

SEQ ID NO: 48 - DNA, 44 bp, synthetic construct
  source 1..44: mol_type = other DNA
ctacacgacgctcttccgatctcggctgacaaaagaagtcctgg

SEQ ID NO: 49 - DNA, 43 bp, synthetic construct
  source 1..43: mol_type = other DNA
agacgtgtgctcttccgatctctattggtcaaggcaaggctgg

SEQ ID NO: 50 - DNA, 42 bp, synthetic construct
  source 1..42: mol_type = other DNA
ctacacgacgctcttccgatctagagagccttccgaaagagg

SEQ ID NO: 51 - DNA, 44 bp, synthetic construct
  source 1..44: mol_type = other DNA
agacgtgtgctcttccgatctggccagaaaagagatatggcatc

SEQ ID NO: 52 - DNA, 20 bp, synthetic construct
  source 1..20: mol_type = other DNA
taccgtacaccactgagacc

SEQ ID NO: 53 - DNA, 21 bp, synthetic construct
  source 1..21: mol_type = other DNA
tcttgggaagtgtaaggaagc

SEQ ID NO: 54 - DNA, 23 bp, synthetic construct
  source 1..23: mol_type = other DNA
ctcccagaacctgagctgctctg

SEQ ID NO: 55 - DNA, 19 bp, synthetic construct
  source 1..19: mol_type = other DNA
ctagccattgtgagctggc

SEQ ID NO: 56 - DNA, 20 bp, synthetic construct
  source 1..20: mol_type = other DNA
acccaatacctcatggatgc

SEQ ID NO: 57 - DNA, 20 bp, synthetic construct
  source 1..20: mol_type = other DNA
ccctggatgtggccatgagg

SEQ ID NO: 58 - DNA, 5822 bp, synthetic construct
  source 1..5822: mol_type = other DNA
tttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcagatctcacgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcggtaccacatgtaagcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcatggctgactaattttttttatttatgcagaggccgaggccggatcctctagcccatgggggccccctcaggggatccacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcagatctgcggccgcactagtgctagactgccatgtcgagggattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataa

SEQ ID NO: 59 - DNA, 4205 bp, synthetic construct
  source 1..4205: mol_type = other DNA
gtgttggaattgtgggaaggagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgacgccaactgctcccccagaggacccagctgtggatctgctaaagaactacatgcagttgggcaagcagcagagagaaagcagagagaagccttacaaggaggtgacagaggatttgctgcacctcaattctctctttggaggagaccagtagtcactgctcatattgaaggacagcctgtagaagtattattggatacaggggctgatgattctattgtaacaggaatagagttaggtccacattataccccaaaaatagtaggaggaataggaggttttattaatactaaagaatacaaaaatgtaaaaatagaagttttaggcaaaaggattaaagggacaatcatgacaggggacactccgattaacatttttggtaggaatttgctaacagctctggggatgtctctaaatcttcccatagctaaggtagagcctgtaaaagtcaccttaaagccaggaaaggttggaccaaaattgaagcagtggccattatcaaaagaaaagatagttgcattaagagaaatctgtgaaaagatggaaaaggatggtcagttggaggaagctcccccgaccaatccatacaacacccccacatttgccataaagaaaaaagataagaacaaatggagaatgctgatagattttagggaactaaatagggtcactcaggactttacagaagtccaattaggaataccacaccctgcaggactagcaaaaaggaaaaggattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagctaaaggaactcttaaatagcatagggttctctaccccagaagagaaattccaaaaagatcccccatttcaatggatggggtacgaattgtggccgacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataagataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagttagactattagcacatgtaatacagaaaataggaaaggaagcaatagtgatctggggacaggtcccaaaattccacttaccagttgagagggatgtatgggaacagtggtggacagactattggcaggtaacctggataccggagtgggattttatctcaacgccaccactagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattagaagcatttctcatggcattgacagactcagggccaaagacaaatattatagtagattcacaatatgttatgggaataataacaggatgccctacagaatcagagagcaggctagttaaccaaataatagaagaaatgattaaaaagtcagaaatttatgtagcatgggtaccagcacacaaaggtataggaggaaaccaagaaatagaccacctagttagtcaggggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaagctatacatgggcaggtaaattcagatctagggacttggcaaatggactgtacccatctagaaggaaaaatagtcatagttgcagtacatgtagctagtggattcatagaagcagaagtaattccacaagagacaggaagacagacagcactatttctgttaaaattggcaggcagatggcctattacacatctacacacagataatggtgctaactttgcctcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagaaatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttccccctacaggaaggaagccatttagaagtacaagggtattggcatttgacaccagaaagagggtggctcagtacttatgcagtgaggataacatggtactcaaggaacttttggacagatgtaacaccagactatgcagacattttactgcatagcacttatttcccttgctttacagcgggagaagtgagaagggccatcaggggagaacaactgctgtcttgctgcaagttcccgagagcacataggtaccaggtaccaagcctacagtacttagcactaaaagtagtaagcgatgtcagatcccagggagagaatcccacctggaaacagtggagaagagacaataggagaggccttcgaatggctaaacagaacagtagaggagataaacagagaggcagtaaaccacctaccaagggagctgattttccaggtttggcaaaggtcttgggaatactggcatgatgaacaagggatgtcacaaagctatgtaaaatacagatacttgtgtttaatgcaaaaggctttatttatgcattgcaagaaaggctgtagatgtctaggggaaggacacggggcaggaggatggagaccaggacctcctcctcctccccctccaggactagcataaatggaagaaagacctccggaaaatgaaggcccacaaagggaaccatgggatgaatgggtagtggaggttctggaagaattgaaagaagaagctttaaaacattttgatcctcgcttgctaactgcacttggtaatcatatctataatagacatggagacacccttgagggagcaggagaactcattagaatcctccaacgagcgctcttcatgcattttagaggcggatgcaaccactccagaatcggccaacctgggggaggaaatcctctctcaactataccgccctcttga

SEQ ID NO: 60 - RNA, 161 nt, synthetic construct; identical to SEQ ID NO: 7
  source 1..161: mol_type = other RNA

SEQ ID NO: 61 - RNA, 100 nt, synthetic construct; identical to SEQ ID NO: 24
  source 1..100: mol_type = other RNA

SEQ ID NO: 62 - RNA, 171 nt, synthetic construct; identical to SEQ ID NO: 9
  source 1..171: mol_type = other RNA

SEQ ID NO: 63 - RNA, 100 nt, synthetic construct; identical to SEQ ID NO: 25
  source 1..100: mol_type = other RNA

SEQ ID NO: 64 - RNA, 100 nt, synthetic construct; identical to SEQ ID NO: 26
  source 1..100: mol_type = other RNA

SEQ ID NO: 65 - RNA, 189 nt, synthetic construct
  source 1..189: mol_type = other RNA
agatgactatctttaatgtcgttttagagctagaaatagcaagttaaaataaggctagtccgttatcaacttgaaaaagtggcaccgagtcggtgcattacacctgcagctctcattttccttatattaaagatagtcatcccaccatacgcggttctatctagttacgcgttaaaccaactagaattt

SEQ ID NO: 66 - RNA, 100 nt, synthetic construct
  source 1..100: mol_type = other RNA
caggacggtcacctttgggggttttagagctagaaatagcaagttaaaataaggctagtccgttatcaacttgaaaaagtggcaccgagtcggtgctttt

SEQ ID NO: 67 - DNA, 6354 bp, synthetic construct
  source 1..6354: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 68 - DNA, 6396 bp, synthetic construct
  source 1..6396: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggactaa

SEQ ID NO: 69 - DNA, 72 bp, synthetic construct
  source 1..72: mol_type = other DNA
tatagcatccaagctgctacagaagaggaacctcaaaacgataatgtgagttctgtaattcagcatatggat

SEQ ID NO: 70 - protein, 15 aa, synthetic construct
YSIQAATEEEPQNDN

SEQ ID NO: 71 - DNA, 72 bp, synthetic construct
  source 1..72: mol_type = other DNA
tatagcatcagggctgctacagaagaggaacctcaaaacgataatgtgagttctgtaattcagcatatggat

SEQ ID NO: 72 - protein, 15 aa, synthetic construct
YSIRAATEEEPQNDN

SEQ ID NO: 73 - DNA, 20 bp, unidentified
  source 1..20: mol_type = unassigned DNA
catccaagctgctacagaag

SEQ ID NO: 74 - DNA, 20 bp, synthetic construct
  source 1..20: mol_type = other DNA
catccaagctgctacagaag

SEQ ID NO: 75 - DNA, 33 bp, synthetic construct
  source 1..33: mol_type = other DNA
tatagcatccaagctgctacagaagaggaacct

SEQ ID NO: 76 - protein, 11 aa, synthetic construct
YSIQAATEEEP

SEQ ID NO: 77 - DNA, 33 bp, unidentified; identical to SEQ ID NO: 75
  source 1..33: mol_type = unassigned DNA

SEQ ID NO: 78 - DNA, 33 bp, synthetic construct
  source 1..33: mol_type = other DNA
tatagcatcagagctgctacagaagaggaacct

SEQ ID NO: 79 - DNA, 33 bp, synthetic construct
  source 1..33: mol_type = other DNA
tatagcatcagggctgctacagaagaggaacct

SEQ ID NO: 80 - DNA, 33 bp, synthetic construct
  source 1..33: mol_type = other DNA
tatagcattagggctgctacagaagaggaacct

SEQ ID NO: 81 - DNA, 33 bp, synthetic construct
  source 1..33: mol_type = other DNA
cgctccgtggccttagctgtgctcgcgctactc

SEQ ID NO: 82 - protein, 11 aa, synthetic construct
RSVALAVLALL

SEQ ID NO: 83 - DNA, 33 bp, unidentified; identical to SEQ ID NO: 81
  source 1..33: mol_type = unassigned DNA

SEQ ID NO: 84 - DNA, 33 bp, synthetic construct
  source 1..33: mol_type = other DNA
cgctccgtggcctgagctgtgctcgcgctactc

SEQ ID NO: 85 - DNA, 33 bp, synthetic construct
  source 1..33: mol_type = other DNA
cgctccgtggcgtgagctgtgctcgcgctactc

SEQ ID NO: 86 - DNA, 33 bp, synthetic construct
  source 1..33: mol_type = other DNA
cgctccgtggggtgagctgtgctcgcgctactc

SEQ ID NO: 87 - DNA, 33 bp, synthetic construct
  source 1..33: mol_type = other DNA
cgctccgtggggtaggctgtgctcgcgctactc

SEQ ID NO: 88 - DNA, 33 bp, synthetic construct
  source 1..33: mol_type = other DNA
cttgggggccccttccccacactatctcaatgc

SEQ ID NO: 89 - DNA, 33 bp, unidentified; identical to SEQ ID NO: 88
  source 1..33: mol_type = unassigned DNA

SEQ ID NO: 90 - DNA, 33 bp, synthetic construct
  source 1..33: mol_type = other DNA
cttgggggcccattctccacactatctcaatgc

SEQ ID NO: 91 - DNA, 33 bp, synthetic construct
  source 1..33: mol_type = other DNA
cttgggggcccgttctccacactatctcaatgc

SEQ ID NO: 92 - DNA, 33 bp, synthetic construct
  source 1..33: mol_type = other DNA
cttgggggccggttgtccacactatctcaatgc

SEQ ID NO: 93 - DNA, 33 bp, synthetic construct
  source 1..33: mol_type = other DNA
cttcctggagcctgtgataaaagcaactgttag

SEQ ID NO: 94 - DNA, 33 bp, unidentified; identical to SEQ ID NO: 93
  source 1..33: mol_type = unassigned DNA

SEQ ID NO: 95 - DNA, 33 bp, synthetic construct
  source 1..33: mol_type = other DNA
cttcctggagcctgccataaaagcaactgttag

SEQ ID NO: 96 - DNA, 33 bp, synthetic construct
  source 1..33: mol_type = other DNA
cttcctggagggtgccataaaagcaactgttag

SEQ ID NO: 97 - DNA, 120 bp, synthetic construct
  source 1..120: mol_type = other DNA
gcatccaagctgctacagaagaggaacctcaaaacgataatgtgagttctgtaattcagcatatggatttgtagtacacatcagatatcttctccgtctttgtctcccacttcttctcaa

SEQ ID NO: 98 - DNA, 120 bp, synthetic construct
  source 1..120: mol_type = other DNA
gcatcagggctgctacagaagaggaacctcaaaacgataatgtgagttctgtaattcagcatatggatttgtagtacacatcagatatcttctccgtctttgtctcccacttcttctcaa

SEQ ID NO: 99 - DNA, 120 bp, synthetic construct
  source 1..120: mol_type = other DNA
gcatcagggctgctacagaagaggaacctcaaaacgataatgtgagttctgtaattcagctacagaagaggaacctcaaaacgataatgtgagttctgtaattcagcatatggatttgta

SEQ ID NO: 100 - DNA, 120 bp, synthetic construct
  source 1..120: mol_type = other DNA
gcatcagggctgctacagaagaggaacctcaaaacgataatgtgagttctgtaattcagggctgctacagaagaggaacctcaaaacgataatgtgagttctgtaattcagcatatggat

SEQ ID NO: 101 - DNA, 120 bp, synthetic construct
  source 1..120: mol_type = other DNA
gcatcagggctgctacagaagaggaacctcaaaacgataatgtgagttctgtaattcagaagaggaacctcaaaacgataatgtgagttctgtaattcagcatatggatttgtagtacac

SEQ ID NO: 102 - DNA, 120 bp, synthetic construct
  source 1..120: mol_type = other DNA
gcatccaagctgctacagaagaggaacctcaaaacgataatgtgagttctgtaattcagaagaggaacctcaaaacgataatgtgagttctgtaattcagcatatggatttgtagtacac

SEQ ID NO: 103 - DNA, 120 bp, synthetic construct
  source 1..120: mol_type = other DNA
gcatccaagctgctacagaagaggaacctcaaaacgataatgtgagttctgtaattcagctacagaagaggaacctcaaaacgataatgtgagttctgtaattcagcatatggatttgta

SEQ ID NO: 104 - DNA, 120 bp, synthetic construct
  source 1..120: mol_type = other DNA
gcatccaagctgctacagaagaggaacctcaaaacgataatgtgagttctgtaattcagggctgctacagaagaggaacctcaaaacgataatgtgagttctgtaattcagcatatggat

SEQ ID NO: 105 - DNA, 120 bp, synthetic construct
  source 1..120: mol_type = other DNA
atgtgagttctgctacagaagaggaacctcaaaacgataatgtgagttctgtaattcagcatatggatttgtagtacacatcagatatcttctccgtctttgtctcccacttcttctcaa

SEQ ID NO: 106 - DNA, 120 bp, synthetic construct
  source 1..120: mol_type = other DNA
gcatccaagctgctacagaagaggaccctcaaaacgataatgtgagttctgtaattcagcatatggatttgtagtacacatcagatatcttctccgtctttgtctcccacttcttctcaa

SEQ ID NO: 107 - DNA, 120 bp, synthetic construct
  source 1..120: mol_type = other DNA
catcaggggctgctacagaagaggaacctcaaaacgataatgtgagttctgtaattcagcatatggatttgtagtacacatcagatatcttctccgtctttgtctcccacttcttctcaa

SEQ ID NO: 108 - DNA, 120 bp, synthetic construct
  source 1..120: mol_type = other DNA
gcatcagggctgctacagaagaggaccctcaaaacgataatgtgagttctgtaattcagcatatggatttgtagtacacatcagatatcttctccgtctttgtctcccacttcttctcaa

SEQ ID NO: 109 - DNA, 120 bp, synthetic construct
  source 1..120: mol_type = other DNA
gcatcagggctgctacagaagaggaacctcaaaacgataatgtgagttctgtaattcagaggaacctcaaaacgataatgtgagttctgtaattcagcatatggatttgtagtacacatc

SEQ ID NO: 110 - DNA, 120 bp, synthetic construct
  source 1..120: mol_type = other DNA
gcatccaagctgctacagaagaggaacctcaaaacgataatgtgagttctgtaattcagaggaacctcaaaacgataatgtgagttctgtaattcagcatatggatttgtagtacacatc

SEQ ID NO: 111 - DNA, 120 bp, synthetic construct
  source 1..120: mol_type = other DNA
gcatcagggctgctacagaagaggaacctcaaaacgataatgtgagttctgtaattcagctgctacagaagaggaacctcaaaacgataatgtgagttctgtaattcagcatatggattt

SEQ ID NO: 112 - DNA, 120 bp, synthetic construct
  source 1..120: mol_type = other DNA
aatgtgagttctgtacagaagaggaacctcaaaacgataatgtgagttctgtaattcagcatatggatttgtagtacacatcagatatcttctccgtctttgtctcccacttcttctcaa

SEQ ID NO: 113 - DNA, 120 bp, synthetic construct
  source 1..120: mol_type = other DNA
agttcagggctgctacagaagaggaacctcaaaacgataatgtgagttctgtaattcagcatatggatttgtagtacacatcagatatcttctccgtctttgtctcccacttcttctcaa

SEQ ID NO: 114 - DNA, 120 bp, synthetic construct
  source 1..120: mol_type = other DNA
cgataatgtgagctacagaagaggaacctcaaaacgataatgtgagttctgtaattcagcatatggatttgtagtacacatcagatatcttctccgtctttgtctcccacttcttctcaa

SEQ ID NO: 115 - DNA, 119 bp, synthetic construct
  source 1..119: mol_type = other DNA
gcatcagggctgctacagaagaggaacctcaaaacgataatgtgagttctgtaatcagggctgctacagaagaggaacctcaaaacgataatgtgagttctgtaattcagcatatggat

SEQ ID NO: 116 - DNA, 120 bp, synthetic construct
  source 1..120: mol_type = other DNA
gcatccaagctgctacagaagaggaacctcaaaacgataatgtgagttctgtaattcagctgctacagaagaggaacctcaaaacgataatgtgagttctgtaattcagcatatggattt

SEQ ID NO: 117 - DNA, 120 bp, synthetic construct
  source 1..120: mol_type = other DNA
cgataatgtgagttctgtaagaggaacctcaaaacgataatgtgagttctgtaattcagcatatggatttgtagtacacatcagatatcttctccgtctttgtctcccacttcttctcaa

SEQ ID NO: 118 - DNA, 120 bp, synthetic construct
  source 1..120: mol_type = other DNA
acgataatgtgagttctgaagaggaacctcaaaacgataatgtgagttctgtaattcagcatatggatttgtagtacacatcagatatcttctccgtctttgtctcccacttcttctcaa

SEQ ID NO: 119 - DNA, 120 bp, synthetic construct
  source 1..120: mol_type = other DNA
ctcaaaacgataatgtgagttctgtacctcaaaacgataatgtgagttctgtaattcagcatatggatttgtagtacacatcagatatcttctccgtctttgtctcccacttcttctcaa

SEQ ID NO: 120 - DNA, 120 bp, synthetic construct
  source 1..120: mol_type = other DNA
tcaaaacgataatgtgagttctgtaacctcaaaacgataatgtgagttctgtaattcagcatatggatttgtagtacacatcagatatcttctccgtctttgtctcccacttcttctcaa

SEQ ID NO: 121 - DNA, 120 bp, synthetic construct
  source 1..120: mol_type = other DNA
gcatcagggctgctacagaagaggaacctcaaaacgataatgtgagttctgtagggctgctacagaagaggaacctcaaaacgataatgtgagttctgtaattcagcatatggatttgta

SEQ ID NO: 122 - DNA, 120 bp, synthetic construct
  source 1..120: mol_type = other DNA
gtatcagggctgctacagaagaggaacctcaaaacgataatgtgagttctgtaattcagcatatggatttgtagtacacatcagatatcttctccgtctttgtctcccacttcttctcaa

SEQ ID NO: 123 - DNA, 120 bp, synthetic construct
  source 1..120: mol_type = other DNA
agaggaacctcaaaacgataatgtgagttcaaaacgataatgtgagttctgtaattcagcatatggatttgtagtacacatcagatatcttctccgtctttgtctcccacttcttctcaa

SEQ ID NO: 124 - DNA, 120 bp, synthetic construct
  source 1..120: mol_type = other DNA
gagttctggctgctacagaagaggaacctcaaaacgataatgtgagttctgtaattcagcatatggatttgtagtacacatcagatatcttctccgtctttgtctcccacttcttctcaa

SEQ ID NO: 125 - DNA, 120 bp, synthetic construct
  source 1..120: mol_type = other DNA
gcatcagtgctgctacagaagaggaacctcaaaacgataatgtgagttctgtaattcagcatatggatttgtagtacacatcagatatcttctccgtctttgtctcccacttcttctcaa

SEQ ID NO: 126 - DNA, 120 bp, synthetic construct
  source 1..120: mol_type = other DNA
ataatgtgagttctacagaagaggaacctcaaaacgataatgtgagttctgtaattcagcatatggatttgtagtacacatcagatatcttctccgtctttgtctcccacttcttctcaa

SEQ ID NO: 127 - DNA, 119 bp, synthetic construct
  source 1..119: mol_type = other DNA
gcatccaagctgctacagaagaggaacctcaaaacgataatgtgagttctgtaatcagggctgctacagaagaggaacctcaaaacgataatgtgagttctgtaattcagcatatggat

SEQ ID NO: 128 - DNA, 120 bp, synthetic construct
  source 1..120: mol_type = other DNA
tcaaaacgataatgtgagaagaggaacctcaaaacgataatgtgagttctgtaattcagcatatggatttgtagtacacatcagatatcttctccgtctttgtctcccacttcttctcaa

SEQ ID NO: 129 - DNA, 120 bp, synthetic construct
  source 1..120: mol_type = other DNA
gcatccaatctgctacagaagaggaacctcaaaacgataatgtgagttctgtaattcagcatatggatttgtagtacacatcagatatcttctccgtctttgtctcccacttcttctcaa

SEQ ID NO: 130 - DNA, 120 bp, synthetic construct
  source 1..120: mol_type = other DNA
acgataatgtgagttctgtagaggaacctcaaaacgataatgtgagttctgtaattcagcatatggatttgtagtacacatcagatatcttctccgtctttgtctcccacttcttctcaa

SEQ ID NO: 131 - DNA, 120 bp, synthetic construct
  source 1..120: mol_type = other DNA
acgataatgtgagttcagaagaggaacctcaaaacgataatgtgagttctgtaattcagcatatggatttgtagtacacatcagatatcttctccgtctttgtctcccacttcttctcaa

SEQ ID NO: 132 - DNA, 120 bp, synthetic construct
  source 1..120: mol_type = other DNA
aaaacgataatgtgagttctgaggaacctcaaaacgataatgtgagttctgtaattcagcatatggatttgtagtacacatcagatatcttctccgtctttgtctcccacttcttctcaa

SEQ ID NO: 133 - DNA, 120 bp, synthetic construct
  source 1..120: mol_type = other DNA
tcatcagggctgctacagaagaggaacctcaaaacgataatgtgagttctgtaattcagcatatggatttgtagtacacatcagatatcttctccgtctttgtctcccacttcttctcaa

SEQ ID NO: 134 - DNA, 120 bp, synthetic construct
  source 1..120: mol_type = other DNA
ataatgtgagttctgtagaagaggaacctcaaaacgataatgtgagttctgtaattcagcatatggatttgtagtacacatcagatatcttctccgtctttgtctcccacttcttctcaa

SEQ ID NO: 135 - DNA, 120 bp, synthetic construct
  source 1..120: mol_type = other DNA
gaacctcaaaacgataatgtgaggaacctcaaaacgataatgtgagttctgtaattcagcatatggatttgtagtacacatcagatatcttctccgtctttgtctcccacttcttctcaa

SEQ ID NO: 136 - DNA, 120 bp, synthetic construct
  source 1..120: mol_type = other DNA
cctcaaaacgataatgtgaagaggaacctcaaaacgataatgtgagttctgtaattcagcatatggatttgtagtacacatcagatatcttctccgtctttgtctcccacttcttctcaa

SEQ ID NO: 137 - DNA, 119 bp, synthetic construct; identical to SEQ ID NO: 115
  source 1..119: mol_type = other DNA

SEQ ID NO: 138 - DNA, 120 bp, synthetic construct
  source 1..120: mol_type = other DNA
taatcagggctgctacagaagaggaacctcaaaacgataatgtgagttctgtaattcagcatatggatttgtagtacacatcagatatcttctccgtctttgtctcccacttcttctcaa

SEQ ID NO: 139 - DNA, 120 bp, synthetic construct; identical to SEQ ID NO: 103
  source 1..120: mol_type = other DNA

SEQ ID NO: 140 - DNA, 120 bp, synthetic construct; identical to SEQ ID NO: 113
  source 1..120: mol_type = other DNA

SEQ ID NO: 141 - DNA, 120 bp, synthetic construct
  source 1..120: mol_type = other DNA
aggaacctcaaaacgataatgtgagttctcaaaacgataatgtgagttctgtaattcagcatatggatttgtagtacacatcagatatcttctccgtctttgtctcccacttcttctcaa

SEQ ID NO: 142 - DNA, 120 bp, synthetic construct
  source 1..120: mol_type = other DNA
tcaaaacgataatgtgagttctgtaacctcaaaaccataatgtgagttctgtaattcagcatatggatttgtagtacacatcagatatcttctccgtctttgtctcccacttcttctcaa

SEQ ID NO: 143 - DNA, 120 bp, synthetic construct
  source 1..120: mol_type = other DNA
tcaaaacgataatgtgagttctggaacctcaaaacgataatgtgagttctgtaattcagcatatggatttgtagtacacatcagatatcttctccgtctttgtctcccacttcttctcaa

SEQ ID NO: 144 - DNA, 120 bp, synthetic construct
  source 1..120: mol_type = other DNA
atgtgagggctgctacagaagaggaacctcaaaacgataatgtgagttctgtaattcagcatatggatttgtagtacacatcagatatcttctccgtctttgtctcccacttcttctcaa

SEQ ID NO: 145 - DNA, 120 bp, synthetic construct
  source 1..120: mol_type = other DNA
agttctgggctgctacagaagaggaacctcaaaacgataatgtgagttctgtaattcagcatatggatttgtagtacacatcagatatcttctccgtctttgtctcccacttcttctcaa

SEQ ID NO: 146 - DNA, 120 bp, synthetic construct
  source 1..120: mol_type = other DNA
gcatcagggctgctacagaagaggaacctcaaaacgataatgtgagttctgtaattcagcagaagaggaacctcaaaacgataatgtgagttctgtaattcagcatatggatttgtagta

SEQ ID NO: 147 - DNA, 120 bp, synthetic construct
  source 1..120: mol_type = other DNA
gcatcagggctgctacagaagaggaacctcaaaacgataatgtgagttctgtaatacagaagaggaacctcaaaacgataatgtgagttctgtaattcagcatatggatttgtagtacac

SEQ ID NO: 148 - DNA, 120 bp, synthetic construct
  source 1..120: mol_type = other DNA
tgagttctgctgctacagaagaggaacctcaaaacgataatgtgagttctgtaattcagcatatggatttgtagtacacatcagatatcttctccgtctttgtctcccacttcttctcaa

SEQ ID NO: 149 - DNA, 60 bp, unidentified
  source 1..60: mol_type = unassigned DNA
ctcaaacagacaccatggtgcacctgactcctgaggagaagtctgccgttactgccctgt

SEQ ID NO: 150 - DNA, 60 bp, synthetic construct
  source 1..60: mol_type = other DNA
ctcaaacagacaccatggtgcacctgactcctgaagagaagtctgccgttactgccctgt

SEQ ID NO: 151 - DNA, 60 bp, synthetic construct; identical to SEQ ID NO: 149
  source 1..60: mol_type = other DNA

SEQ ID NO: 152 - DNA, 60 bp, synthetic construct
  source 1..60: mol_type = other DNA
ctcaaacagacaccatggtgcacctgactccctgaagagaagtctgccgttactgccctg

SEQ ID NO: 153 - DNA, 60 bp, synthetic construct
  source 1..60: mol_type = other DNA
ctcaaacagacaccctggtgcacctgactcctgaagagaagtctgccgttactgccctgt

SEQ ID NO: 154 - DNA, 57 bp, synthetic construct
  source 1..57: mol_type = other DNA
ctcaaacagacaccatggtgcacctgactcctgaagagaagtctgcaccgactcggt

SEQ ID NO: 155 - DNA, 60 bp, synthetic construct
  source 1..60: mol_type = other DNA
ctcaaacagacaccctggtgcacctgactcctgaggagaagtctgccgttactgccctgt

SEQ ID NO: 156 - DNA, 60 bp, synthetic construct
  source 1..60: mol_type = other DNA
ctcaaacagacaccatggtgcacctgactcctgaagagaactgaggagaagtctgccgtt

SEQ ID NO: 157 - DNA, 60 bp, synthetic construct
  source 1..60: mol_type = other DNA
ctcaaacagacaccatggtgcacctgactcctgaagagaagtcaggaagtctgccgttac

SEQ ID NO: 158 - DNA, 59 bp, synthetic construct
  source 1..59: mol_type = other DNA
ctcaaacagacaccatggtgcacctgactcctaagagaagtctgccgttactgccctgt

SEQ ID NO: 159 - DNA, 60 bp, unidentified
  source 1..60: mol_type = unassigned DNA
ttatcgttttgaggttcctcttctgtagcagcttggatgctataagccaagaaacaaaga

SEQ ID NO: 160 - DNA, 60 bp, synthetic construct; identical to SEQ ID NO: 159
  source 1..60: mol_type = other DNA

SEQ ID NO: 161 - DNA, 60 bp, synthetic construct
  source 1..60: mol_type = other DNA
ttatcgttttgaggttcctcttctgtagcagccctaatgctataagccaagaaacaaaga

SEQ ID NO: 162 - DNA, 60 bp, synthetic construct
  source 1..60: mol_type = other DNA
ttatcgttttgagggtcctcttctgtagcagcttggatgctataagccaagaaacaaaga

SEQ ID NO: 163 - DNA, 60 bp, synthetic construct
  source 1..60: mol_type = other DNA
ttatcgttttgagggtcctcttctgtagcagccctaatgctataagccaagaaacaaaga

SEQ ID NO: 164 - DNA, 120 bp, synthetic construct; identical to SEQ ID NO: 101
  source 1..120: mol_type = other DNA

(19) United States
(12) Patent Application Publication
(10) Pub. No.: US 2026/0193679 A1
(43) Pub. Date: Jul. 9, 2026

(54) SYSTEMS AND METHODS FOR MODIFYING A POLYNUCLEOTIDE

(71) Applicant: The Children's Medical Center Corporation, Boston, MA (US)

(72) Inventors: Daniel E. Bauer, Boston, MA (US); Sébastien Levesque, Boston, MA (US)

(73) Assignee: The Children's Medical Center Corporation, Boston, MA (US)

(21) Appl. No.: 19/417,758

(22) Filed: Dec. 12, 2025

Related U.S. Application Data
(63) Continuation of application No. PCT/US2024/033736, filed on Jun. 13, 2024.
(60) Provisional application No. 63/521,012, filed on Jun. 14, 2023.
(60) Provisional application No. 63/544,549, filed on Oct. 17, 2023.

(51) Int. Cl. C12N 15/90; C07K 14/005; C12N 9/12; C12N 9/22; C12N 15/11

(52) U.S. Cl. CPC C12N 15/907; C07K 14/005; C12N 9/127; C12N 9/1276; C12N 9/226; C12N 15/11; C12Y 207/07048; C12Y 207/07049; C07K 2319/00; C12N 2310/20; C12N 2740/15022; C12N 2740/16022

(57) ABSTRACT

The present disclosure features systems and methods for modifying a target polynucleotide.

### CROSS-REFERENCE TO RELATED APPLICATIONS

This application is a continuation under 35 U.S.C. § 111 (a) of PCT International Patent Application No. PCT/US2024/033736, filed Jun. 13, 2024, designating the United States and published in English, claims priority to and the benefit of U.S. Provisional Application Nos. 63/521,012, filed Jun. 14, 2023, and 63/544,549, filed Oct. 17, 2023, each of which is incorporated herein by reference in its entirety.

### STATEMENT OF RIGHTS TO INVENTIONS MADE UNDER FEDERALLY SPONSORED RESEARCH

This invention was made with government support under grant No. HL 170629 awarded by the National Institutes of Health. The government has certain rights in the invention.

### SEQUENCE LISTING

The instant application contains a Sequence Listing which has been submitted electronically as an XML file and is hereby incorporated by reference in its entirety. The Sequence Listing, created Jun. 27, 2024, is named “167705-033202PCT_SL.xml” and is 275,926 bytes in size.

### BACKGROUND OF THE INVENTION

Reverse transcriptases (RTs) are enzymes that can generate a complementary strand of DNA (cDNA) from RNA. Classical retroviral RTs have been improved by protein engineering. Engineered RTs fused to a CRISPR/Cas9 nickase have recently shown great potential as tools to manipulate eukaryotic genomes. However, efficiency of these RT-dependent gene editors remains low and variable, particularly in hematopoietic stem and progenitor cells. Therefore, there is a need for improved systems and methods for RT-dependent gene editing systems with increased efficiency.

### SUMMARY OF THE INVENTION

As described below, the present disclosure features systems, compositions, and methods for enhancing gene editing technologies that involve the use of a reverse transcriptase (e.g., Prime editing, gene writing, retrotransposition, and retrons).

In an aspect, the present disclosure provides a method for enhancing prime editing in a hematopoietic stem and progenitor cell (HSPC). The method involves contacting the HSPC with the following components under conditions permissive for genome editing, the components including
a programmable DNA binding protein;
a DNA nickase;
a polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide or a primer binding sequence that binds the nicked single stranded DNA and a template sequence to specify the edit to be introduced by the polymerase; and
a VPX polypeptide or a polynucleotide encoding a VPX polypeptide.

In another aspect, the present disclosure provides a method for enhancing prime editing in a hematopoietic stem and progenitor cell (HSPC). The method involves contacting the HSPC with the following components under conditions permissive for genome editing, the components including
a programmable DNA binding protein;
a DNA nickase;
a polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide; and
exogenous deoxynucleosides or deoxynucleotides.

In another aspect, the present disclosure provides a method of modifying an HSPC genome. The method involves contacting the HSPC with the following components under conditions permissive for genome editing, the components including
a programmable DNA binding protein;
a DNA nickase;
a polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide; and
a VPX polypeptide or a polynucleotide encoding a VPX polypeptide.

In another aspect, the present disclosure provides a method of modifying an HSPC genome. The method involves contacting the HSPC with the following components under conditions permissive for genome editing, the components including
a programmable DNA binding protein;
a DNA nickase;
a polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide; and
exogenous deoxynucleosides or deoxynucleotides.

In another aspect, the present disclosure provides an HSPC. The HSPC includes:
a programmable DNA binding protein;
a DNA nickase;
a polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide; and
a VPX polypeptide or a polynucleotide encoding a VPX polypeptide.

In another aspect, the present disclosure provides a method for enhancing prime editing in a hematopoietic stem and progenitor cell (HSPC). The method involves contacting the HSPC with the following components under conditions permissive for genome editing, the components including
a programmable DNA binding protein;
a DNA nickase;
a DNA polymerase;
a prime editing guide RNA including a gRNA target sequence that binds target DNA, a primer binding sequence that primes DNA synthesis, and a template containing an edited RNA sequence, where the guide RNA is capable of directing the fusion protein to a target sequence in a polynucleotide; and
a VPX polypeptide or a polynucleotide encoding a VPX polypeptide,
where the template is designed to evade mismatch repair.

In another aspect, the present disclosure provides a method for enhancing prime editing in a hematopoietic stem and progenitor cell (HSPC), the method comprising contacting the HSPC with the following components under conditions permissive for genome editing, the components comprising
a programmable DNA binding protein;
a DNA nickase;
a DNA polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide;
a VPX polypeptide or a polynucleotide encoding a VPX polypeptide; and
a mismatch repair inhibitor.

In another aspect, the present disclosure provides a method for enhancing prime editing in a human hematopoietic stem and progenitor cell (HSPC). The method involves contacting the HSPC with the following components under conditions permissive for genome editing, the components including
a programmable DNA binding protein;
a DNA nickase;
a reverse transcriptase;
a pair of prime editing guide RNAs each including a gRNA target sequence that binds target DNA, a primer binding sequence that primes DNA synthesis, and a template containing an edited RNA sequence, where the guide RNA is capable of directing the fusion protein to a target sequence in a polynucleotide;
a VPX polypeptide or a polynucleotide encoding a VPX polypeptide,
where the template in each prime editing guide RNA is complementary to the template in the other prime editing guide RNA.

In any of the above aspects, or embodiments thereof, the programmable DNA binding protein includes a Cas9 having nickase activity.

In any of the above aspects, or embodiments thereof, the method further involves contacting the HSPC with exogenous deoxynucleosides or deoxynucleotides.

In any of the above aspects, or embodiments thereof, the method further involves contacting the HSPC with a VPX polypeptide or a polynucleotide encoding a VPX polypeptide.

In any of the above aspects, or embodiments thereof, the VPX polypeptide is encapsulated in a virus-like particle (VLP) or is encoded by an mRNA.

In any of the above aspects, or embodiments thereof, the method further involves contacting the HSPC with deoxynucleosides or deoxynucleotides.

In any of the above aspects, or embodiments thereof, the polymerase is a DNA-dependent or an RNA-dependent DNA polymerase. In any of the above aspects, or embodiments thereof, the RNA-dependent polymerase is a reverse transcriptase. In any of the above aspects, or embodiments thereof, the reverse transcriptase includes a mutation that increases dNTPs affinity. In any of the above aspects, or embodiments thereof, the mutation is V223M.

In any of the above aspects, or embodiments thereof, the polymerase is an RNA-dependent RNA polymerase. In any of the above aspects, or embodiments thereof, the RNA-dependent RNA polymerase is nsp12.

In any of the above aspects, or embodiments thereof, the HSPC is a human HSPC.

In any of the above aspects, or embodiments thereof, the mismatch repair inhibitor is a dominant negative human MutL homolog (MLH1) peptide.

In any of the above aspects, or embodiments thereof, the pair of prime editing guide RNAs is designed to prevent mismatches when a genome of the HSPC is edited by the components.

In any of the above aspects, or embodiments thereof, the template is designed to evade mismatch repair.

Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant an edited cell, a gene editing system, a polypeptide, polynucleotide, or small molecule.

By “ameliorate” is meant decrease, suppress, attenuate, diminish, arrest, or stabilize the development or progression of a disease. In some embodiments, the disease is characterized by a deleterious alteration in the genome of a cell relative to a reference genome that does not comprise said deleterious alteration.

By “alteration” is meant a change in the structure, expression levels or activity of a polynucleotide or polypeptide as detected by standard art known methods such as those described herein. In some embodiments, the alteration is an insertion, deletion, or chromosomal translocation. In some embodiments, the alteration is a change in the sequence of a polynucleotide relative to a reference sequence. In some embodiments, the alteration can be an increase or a decrease in activity, for example. As used herein, an alteration includes a 10% change in expression levels, a 25% change, a 40% change, and a 50% or greater change in expression levels.

By “analog” is meant a molecule that is not identical to a reference molecule, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. In embodiments, a Cas9 nuclease has nickase activity. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torques I (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected. In some embodiments, the analyte is the presence of an edit. In some embodiments, the efficiency of editing is characterized. Means of characterizing editing include TIDE and BEAT.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In particular embodiments, the disease is associated with a deleterious genetic alteration. Examples of diseases include neoplasias, hemoglobinopathies (e.g., sickle cell disease, beta-thalassemia, alpha-thalassemia), enzymopathies (e.g., pyruvate kinase deficiency), membranopathies (e.g., hereditary spherocytosis), inherited bone marrow-failure disorders (e.g., Fanconi anemia, dyskeratosis congenita, severe congenital neutropenia, Diamond-Blackfan anemia, congenital amegakaryocytic thrombocytopenia), disorders of leukocyte function (e.g., chronic granulomatous disease), inherited immunity disorders (e.g., severe combined immunodeficiency), disorders of the complement system (e.g., atypical hemolytic uremic syndrome), inherited metabolic disorders (e.g., phenylketonuria), inherited lung diseases (e.g., cystic fibrosis), inherited liver disorders (e.g., hereditary tyrosinemia), bleeding disorders (e.g., hemophilia), chronic infectious diseases (e.g., HIV infection), inherited neurological conditions (e.g., Huntington's disease), lysosomal storage disorders (e.g., Mucopolysaccharidosis Type 1), disorders where germline or somatic mutations are associated with disease, disorders in which genetic modification could achieve a therapeutic effect, or any disease treatable by correction of a pathogenic gene variant by an RT-dependent gene editing technology, including those variants listed in the ClinVar database (www.ncbi.nlm.nih.gov/clinvar/).

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “gene editing system” is meant the polypeptides, polynucleotides, and other reagents involved in introducing an alteration to a polynucleotide sequence.

By “guide polynucleotide” is meant a polynucleotide or polynucleotide complex that is specific for a target sequence and that can form a complex with a polynucleotide programmable nucleotide binding domain protein (e.g., Cas9 or Cpf1). In an embodiment, the guide polynucleotide is a guide RNA (gRNA). In another embodiment, a gRNA can exist as a complex of two or more RNAs, or as a single RNA molecule.

By “heterologous,” or “exogenous” is meant a polynucleotide or polypeptide that has been experimentally incorporated to a polynucleotide or polypeptide sequence to which the polynucleotide or polypeptide is not normally found in nature; and/or that has been recombinantly expressed in a cell.

By “Reverse Transcriptase (RT)-dependent gene editing system” is meant any gene editing system that uses reverse transcription from an RNA template to synthesize DNA to alter a target polynucleotide. In embodiments, an RT-dependent gene editing system is used to correct, replace, or insert a new polynucleotide sequence relative to a reference polynucleotide sequence. Exemplary RT-dependent gene editing systems include systems involving prime editing, technologies utilizing retron or engineered retron gene editing, gene editing using a retrotransposon, or Gene Writing™.

By “increase” is meant to alter positively relative to a reference. An increase may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By a “mismatch repair inhibitor” is meant any agent that reduces the activity of the mismatch repair pathway (MMR). The MMR pathway is the main pathway responsible for repair of base-base mismatches and insertion and/or deletion loops that are formed during DNA replication. The canonical human MMR pathway has two major components, both heterodimers, with names originating from their lower organism orthologs, MutS and MutL. Inhibitors of the MMR pathway include any agent which inhibits MutS and/or MutL, such as a dominant negative human MutL homolog (MLH1) peptide.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; I S, T; (f) Q, N; and (g) E, D. In various embodiments, conservative amino acid substitutions can be made to the amino acid sequence of the proteins and polypeptides disclosed herein.

By “prime editing” is meant the use of a programmable DNA binding protein, a DNA nickase (e.g., a CAS domain), a reverse transcriptase, a nucleic acid template including a priming sequence, and a reverse transcriptase template sequence, to introduce an alteration in a polynucleotide relative to a reference polynucleotide. In an embodiment, prime editing includes the use of a fusion protein comprising a CAS domain fused to a reverse transcriptase, a prime editing guide RNA (pegRNA) and a single guide RNA to introduce an alteration in a polynucleotide relative to a reference polynucleotide. In an embodiment, a prime editing target polynucleotide comprises a double stranded DNA molecule having two complementary strands: a first strand that may be referred to as a “target strand” or a “non-edit strand”, and a second strand that may be referred to as a “non-target strand,” or an “edit strand.” Exemplary prime editing systems are described, for example, in U.S. Pat. No. 11,447,770, which is incorporated herein by reference in its entirety.

By “nucleic acid programmable DNA binding protein (napDNAbp)” is meant a protein that forms a complex with (e.g., binds or associates with) a nucleic acid molecule capable of guiding the protein to a specific target sequence. In one embodiment, the napDNABP forms a complex with a guide RNA, gRNAs can exist as a complex of two or more RNAs, or as a single RNA molecule. gRNAs that exist as a single RNA molecule may be referred to as single-guide RNAs (sgRNAs), though “gRNA” is used interchangeabley to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a traerRNA, and comprises a stem-loop structure. See, for example, Jinek et al., Science 337:816-821 (2012), the entire contents of which is incorporated herein by reference.

By “reduce” is meant to alter negatively relative to a reference. A reduction may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In embodiments, a standard or control condition involves the use of an RT-dependent gene editing system in the absence of VPX or exogenous deoxynucleosides.

By “reference sequence” is met a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In some embodiments, the reference sequence is the sequence of a reference genome. In some embodiments, a reference sequence is the sequence of a polynucleotide, gene, or genome prior to editing with a gene editing system described herein.

By “retron” is meant a polynucleotide encoding an RNA comprising msr and msd regions and a reverse transcriptase capable of reverse transcribing the msd into msDNA. In an embodiment, a retron refers to an endogenous bacterial element that generates a single strand of DNA (ssDNA) from a structured noncoding RNA transcript. See Lampson et al., Cytogenet Genome Res. 110 (104): 491-499 (2005) hereby incorporated by reference in its entirety.

By “specifically binds” is meant a polypeptide or polynucleotide that recognizes and binds a target polypeptide or polynucleotide, but which does not substantially recognize and bind other molecules in a sample, for example, a biological sample.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than about 500 mM NaCl and 50 mM trisodium citrate, or about less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In another embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “target site” refers to a nucleotide sequence or nucleobase of interest that is modified. In embodiments, the target site exists within a larger polynucleotide molecule (e.g., DNA, gene, genome).

“Transposons” as used herein are polynucleotides, or fragments thereof, capable of moving within a genome. Transposons are also known as “jumping gene”. There are at least two different classes of transposons: class 1, or retrotransposons, that mobilize via an RNA intermediate and a “copy-and-paste” mechanism, and class II, or DNA transposons, that mobilize via excision integration, or a “cut-and-paste” mechanism (Ivics Nat Methods 6, 415-422 (2009)). Bacterial, lower eukaryotic (e.g. yeast) and invertebrate transposons appear to be largely species specific, and cannot be used for efficient transposition of DNA in vertebrate cells. “Sleeping Beauty” (Ivics et al., Cell 1997 Nov. 14; 91 (4): 501-10), was the first active transposon that was artificially reconstructed by sequence shuffling of inactive TEs from fish. This made it possible to successfully achieve DNA integration by transposition into vertebrate cells, including human cells. Sleeping Beauty is a class II DNA transposon belonging to the Tcl/mariner family of transposons (Ni Genomics Proteomics 2008). In the meantime, additional functional transposons have been identified or reconstructed from different species, including Drosophila, frog and even human genomes, that all have been shown to allow DNA transposition into vertebrate and also human host cell genomes. Each of these transposons have advantages and disadvantages that are related to transposition efficiency, stability of expression, genetic payload capacity etc. Exemplary class II transposases that have been created include Sleeping Beauty, Piggy Bac, Frog Prince, Himarl, Passport, Minos, hAT, Toll, Tol2, AciDs, PIF, Harbinger, Harbinger3-DR, and Hsmarl.

By “transposase” is meant an enzyme that binds the end of a transposon and catalyzes its movement into another part of a genome.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

By “Viral Protein X (VPX) polypeptide” or “Vpx polypeptide” is meant a protein or fragment thereof having at least 85% amino acid sequence identity to the amino acid sequence of GenBank Accession Nos. P89156 or P18099.1 and having SAM domain and HD domain-containing protein 1 (SAMHD1) binding activity.

>sp|P89156|P89156_SIVCZ Vpx protein [Simian
immunodeficiency virus]
(SEQ ID NO: 1)
MSDPRERIPPGNSGEETIEEAFEWLNRTVEGINRAAVNHLPRELIFQVWQ
RSWEYWHDEMGMSESYTKYRYLCLIQKALFMHCKKGCRCLGEGHGAGGWR
TGPPPPPPPGLA
>sp|P18099.1|VPX_HV2BE [Human immunodeficiency
virus 2]
(SEQ ID NO: 2)
MTDPRERVPPGNSGEETIGEAFEWLERTIEALNREAVNHLPRELIFQVWQ
RSWRYWHDEQGMSASYTKYRYLCLMQKAIFTHFKRGCTCWGEDMGREGLE
DQGPPPPPPPGLV

By “VPX” polynucleotide is meant any nucleic acid molecule encoding a VPX polypeptide or fragment thereof. Exemplary full length sequences of VPX polynucleotides are found below.

VPX (SIV)
(SEQ ID NO: 3)
ATGTCAGATCCCAGGGAGAGAATCCCACCTGGAAACAGTGGAGAAGAGACA
ATAGGAGAGGCCTTCGAATGGCTAAACAGAACAGTAGAGGAGATAAACAGA
GAGGCAGTAAACCACCTACCAAGGGAGCTGATTTTCCAGGTTTGGCAAAGG
TCTTGGGAATACTGGCATGATGAACAAGGGATGTCACAAAGCTATGTAAAA
TACAGATACTTGTGTTTAATGCAAAAGGCTTTATTTATGCATTGCAAGAAA
GGCTGTAGATGTCTAGGGGAAGGACACGGGGCAGGAGGATGGAGACCAGGA
CCTCCTCCTCCTCCCCCTCCAGGACTAGCATGA
VPX (HIV2)
(SEQ ID NO: 4)
ATGACAGACCCCAGAGAAAGGGTACCGCCAGGAAACAGTGGAGAAGAGACC
ATTGGAGAGGCCTTCGAGTGGCTAGAGAGGACCATAGAAGCCTTAAACAGG
GAGGCAGTGAACCATCTGCCCCGAGAGCTCATTTTCCAGGTGTGGCAAAGG
TCCTGGAGATATTGGCATGATGAACAAGGGATGTCAGCAAGCTACACAAAG
TATAGATATTTGTGCCTAATGCAAAAAGCTATATTTACACATTTCAAGAGA
GGGTGCACTTGCTGGGGGGAGGACATGGGCCGGGAAGGATTGGAAGACCAA
GGACCTCCCCCTCCTCCCCCTCCAGGTCTAGTCTAA

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about. The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A and FIG. 1B provide a schematic and charts showing that VPX virus-like particles (VLPs) enhance prime editing in human hematopoietic stem and progenitor cells (HSPCs). FIG. 1A provides a schematic representation of the installation of the Q118R mutation at ATP1A1 using the prime editing system 3 (PE3). FIG. 1B shows Prime Editing (PE) quantification as determined by Base Editing Analysis Tool (BEAT) analysis from Sanger sequences. HSPCs were thawed and cultured for 24 hours before electroporation. 5×10⁵ HSPCs were electroporated with 2000 ng PE2 mRNA+200 pmol synthetic ATPIA1-Q118R epegRNA+100 pmol synthetic nick sgRNA using pulse code DS-130. HSPCs were then cultured for 72 hours in the presence or absence of 5×VPX (SIV) VLPs. 1× lentiBOOST™ was used in all conditions. DMEM supplemented with 10% FBS and 1% Pen/Strep was used as a vehicle control for VPX VLPs. Genomic DNA was harvested 3 days post-nucleofection. Figure discloses SEQ ID NOS 69-74 and 74, respectively, in order of appearance.

FIGS. 2A and 2B provide bar graphs showing that VPX virus-like particles (VLPs) and the addition of deoxynucleosides (dNs) enhance prime editing in HSPCs. FIGS. 2A and 2B provide PE and indels quantification as determined by BEAT and Tracking of indels by decomposition (TIDE) analysis from Sanger sequences, and cell viability as determined by trypan blue staining 24 and 72 hours after nucleofection. Cells were thawed and cultured with or without 100 μM each dNs. After 24 hours of culture, 5×10⁵ HSPCs were electroporated with 2000 ng PE2 mRNA+200 pmol synthetic ATPIA1-Q118R epegRNA+100 pmol synthetic nick sgRNA using pulse code DS-130. HSPCs were then cultured for 72 hours in the presence or absence of 100 μM dNs and VPX (SIV) VLPs. 1× lentiBOOST™ was used in all conditions. DMEM supplemented with 10% FBS and 1% Pen/Strep was used as a vehicle control for VPX VLPs. Genomic DNA was harvested 3 days post-nucleofection. FIG. 2B was carried out under the same conditions as in FIG. 2A, but using 1.25×10⁵ cells per nucleofection, and different concentrations of dNs before and after nucleofection.

FIG. 3 provides bar graphs showing that the addition of deoxynucleosides (dNs) enhances prime editing in resting and activated CD3⁺ T cells. PE and indels quantification were determined by BEAT and TIDE analysis from Sanger sequences.

FIGS. 4A and 4B are schematics showing components of Prime Editing (FIG. 4A) and the Prime Editing process (FIG. 4B).

FIGS. 5A-5F provide schematics and plots showing that Vpx and deoxynucleoside (dN) supplementation enhances prime editing in hematopoietic stem and progenitor cells. (FIG. 5A) Schematic representation of the strategies used for the modulation of nucleotide metabolism to increase the concentration of dNTPs available for reverse transcription and prime editing. (FIG. 5B) PE and indels quantification as determined by BEAT and TIDE analysis from Sanger sequencing. 2.5×10⁵ HSPCs were electroporated with PE3max RNAs targeting ATP1A1 and treated with 50 μM of the indicated dNs. Genomic DNA was harvested 3 days post-nucleofection. Data are plotted as mean±SEM from n=3 independent biological replicates performed with CD34⁺ HSPCs from three different donors. (FIG. 5C) TwinPE and indel quantification as determined by CRISPResso2 analysis from amplicon sequencing. HSPCs were cultured (stimulated) or not (quiescent) with cytokines for 24 hours prior to electroporation. 5×10⁵ quiescent or 2.5×10⁵ stimulated HSPCs were electroporated with Vpx mRNA and TwinPEmax RNAs targeting AAVS1 or CCR5 and cultured in the presence or absence of 50 μM each dNs. Genomic DNA was harvested 3 days post-nucleofection. Data are plotted as mean±SEM from n=3-4 independent biological replicates performed with CD34⁺ HSPCs from three to four different donors. (FIG. 5D) Same as in (c) using PEmax or PEmax-V223M mRNA. PE and indel quantification as determined by CRISPResso2 analysis from amplicon sequencing for TwinPE at AAVS1, and by BEAT and TIDE analysis from Sanger sequencing for PE at ATP1A1 and RNF2. Data are plotted as mean±SEM from n=3 independent biological replicates performed with CD34⁺ HSPCs from three different donors. For each of FIGS. 5B-5D, plots are organized as pairs of bars, with each left bar corresponding to PE and each right bar corresponding to indels. (FIG. 5E) Schematic representation of the ddPCR assay designed to measure PE-mediated reverse transcription. (FIG. 5F) Percentage of allele harboring the PE flap relative to a genomic reference as determined by ddPCR. Data are plotted as mean±SEM from n=3 independent biological replicates performed with CD34⁺ HSPCs from three different donors. Each data point represents the average of a technical triplicate. Donor 2, circle. Donor 3, diamond. Donor 4, square. Donor 5, triangle (up). Donor 6, empty diamond. Donor 7, triangle (down).

FIGS. 6A-6D provide schematics and plots showing that modulation of nucleotide metabolism positively interacts with MMR evasion. (FIG. 6A) PE and indel quantification as determined by CRISPResso2 analysis from amplicon sequencing. The MMR evasion strategy based on the installation of additional silent mutations and the epegRNAs used to target the ATP1A1 locus are illustrated. Point mutations are illustrated in orange. 2.5×10⁵ HSPCs were electroporated with PE2max RNAs targeting ATP1A1 and cultured in the presence or absence of 50 μM each dN and 25× in-house Vpx VLPs. Genomic DNA was harvested 3 days post-nucleofection. Data are plotted as mean±SEM from n=3 independent biological replicates performed with CD34⁺ HSPCs from three different donors. (FIG. 6B) Same as in (FIG. 6A), but HSPCs were electroporated with Vpx mRNA and PE2max RNAs targeting B2M. Data are plotted as mean±SEM from n=3 independent biological replicates performed with CD34⁺ HSPCs from three different donors. (FIG. 6C) Same as in (FIG. 6B), but HSPCs were electroporated with Vpx mRNA and PE3max RNAs targeting the HBG1/HBG2 promoters. Data are plotted as mean±SEM from n=3 independent biological replicates performed with CD34⁺ HSPCs from three different donors. (FIG. 6D) Same as in (FIG. 6B), but HSPCs were electroporated with Vpx mRNA and PE2max RNAs targeting the +58 BCL11A enhancer. Data are plotted as mean±SEM from n=3 independent biological replicates performed with CD34⁺ HSPCs from three different donors. Donor 2, circle. Donor 3, diamond. Donor 6, empty diamond. Donor 7, triangle (down). Figure discloses SEQ ID NOS 75-96, respectively, in order of appearance.

FIGS. 7A-7B provide plots showing that modulation of nucleotide metabolism has minimal impact on prime editing in cancer cell lines. (FIG. 7A) PE and indels quantification as determined by BEAT analysis from Sanger sequencing. K562 cells were electroporated with PE3max vectors targeting ATP1A1 and the indicated Vpx or SAMHD1 vector and cultured with the indicated concentration of each dN for 72 hours. An empty pUC19 vector was used as a negative control to normalize the concentration of DNA in all nucleofections. Genomic DNA was harvested 3 days post-nucleofection. n=2 independent biological replicates performed at different times. (FIG. 7B) Same as in (FIG. 7A) with Jurkat cells.

FIGS. 8A-8D provides allele plots showing that staggered PE3 nicks generate tandem duplications at ATP1A1 in HSPCs. CRISPResso2 amplicon sequencing allele plots after PE3 at ATP1A1. 2.5×10⁵ HSPCs were electroporated with PE3max RNAs targeting ATP1A1 (Q118R_v2, +3 TTG to CCT) and cultured in the presence or absence of 5×Vpx VLPs (GPP) and 50 μM each dN. Genomic DNA was harvested 3 days post-nucleofection. Representative allele plots are from one of three independent biological replicates performed with CD34⁺ HSPCs from three different donors with equivalent results. Insertions are illustrated in squares, and they represent tandem duplications of the genomic sequence found between the two nicking sites. Figure discloses SEQ ID NOS 97, 97-101, 98, 102-120, 98, 121-129, 98, 97, 99, 101, 98, 100, 103, 107, 105, 102, 104, 108, 110, 106, 109, 113, 117, 114, 130-131, 98, 98, 112, 132-133, 111, 128, 134, 126, 135, 98, 136-138, 113, 98, 97, 99, 139, 164, 98, 100, 105, 102, 104, 109, 107-108, 110, 140, 114, 132, 130, 111, 141, 131, 137, 117, 128, 142, 116, 126, 124, 112, 133, 106, 143-145, 98, 122, 98, 97, 99, 101, 98, 100, 103, 105, 104, 107, 108, 102, 109, 113, 111, 117, 144, 112, 110, 135, 146, 98, 98, 98, 133, 137, 121, 143, 132, 134, 145 and 147-148, respectively, in order of appearance.

FIGS. 9A-9C. provide plots showing that modulation of nucleotide metabolism modestly improves PE at HBB. (FIG. 9A) PE and indels quantification as determined by CRISPResso2 analysis from amplicon sequencing. 2.5×10⁵ HSPCs were electroporated with PE3/PE3bmax RNAs targeting HBB and cultured in the presence or absence of 5×Vpx VLPs (GPP) and 50 μM each dNs. Genomic DNA was harvested 3 days post-nucleofection. Data are plotted as mean±SEM from n=3 independent biological replicates performed with CD34⁺ HSPCs from one donor homozygous for the rs713040 allele (donor 2). Each biological replicate is illustrated with the same shade of grey. (FIG. 9B) Same as in (FIG. 9A) but HSPCs were electroporated with PE2max or PE4max (PE2max mRNA+hMLHldn mRNA) RNAs targeting HBB and cultured in the presence or absence of 50 μM each dNs. Where indicated, an equimolar ratio of Vpx mRNA was co-delivered during electroporation. The mRNA molarity was normalized between all conditions with EGFP mRNA. Data are plotted as mean±SEM from n=3 independent biological replicates performed with CD34⁺ HSPCs from three different donors. Donor 2, circle. Donor 6, empty diamond. Donor 7, triangle (down). (FIG. 9C) Representative CRISPResso2 amplicon sequencing allele plots from the experiments shown in (FIG. 9A) and (FIG. 9B), Representative allele plots are from one of three independent biological replicates performed with CD34⁺ HSPCs from three different donors with equivalent results. Reads with >0.2% frequency are shown, and the frequency of indels is indicated for each sample. Figure discloses SEQ ID NOS 149-158, 149, 151, 150, 155, 153, 149, 151, 150, 149, 151 and 150, respectively in order of appearance.

FIGS. 10A-10C provide a schematic and plots showing that modulation of nucleotide metabolism positively interacts with MMR-evading epegRNAs in quiescent HSPCs. (FIG. 10A) Schematic representation of the RNAs used to co-deliver Vpx with PE3max components. The timeline for prime editing in quiescent CD34⁺ HSPCs is illustrated on the right. (FIG. 10B) PE and indels quantification as determined by BEAT and TIDE analysis from Sanger sequencing. 5×10⁵ quiescent HSPCs were electroporated directly after thawing with Vpx mRNA and PE3max RNAs targeting HBB or B2M and cultured in the presence or absence of 50 μM each dN. Genomic DNA was harvested 3 days post-nucleofection. Data are plotted as mean±SEM from n=3 independent biological replicates performed with CD34⁺ HSPCs from three different donors. (FIG. 10C) Same as in (FIG. 10B) for prime editing at ATP1A1 with MMR-evading substitutions. Donor 2, circle. Donor 6, empty diamond. Donor 7, triangle (down).

FIG. 11 provides a plot showing that omitting the nicking sgRNA abrogates indels and tandem duplications at ATP1A1 in HSPCs. CRISPResso2 amplicon sequencing allele plots from FIG. 6A. 2.5×10⁵ HSPCs were electroporated with PE2max RNAs targeting ATP1A1 (Q118R_v3, +3 TTGG to CCTA) and cultured in the presence or absence of 25× in-house Vpx VLPs and 50 μM each dN. Genomic DNA was harvested 3 days post-nucleofection. Representative allele plots are from one of three independent biological replicates performed with CD34⁺ HSPCs from three different donors with equivalent results. Reads with >0.2% frequency are shown, and the frequency of indels is indicated for each sample. Figure discloses SEQ ID NOS 159-162, 159-163, 159-163, 159, 161, 160, 163 and 162, respectively, in order of appearance.

FIGS. 12A-12D provide schematics, plots, and a graph showing a comparison of quantification accuracy between Sanger and amplicon sequencing at ATP1A1 and B2M (related to FIGS. 6 and 7). (FIG. 12A) PE and indel quantification as determined by BEAT and TIDE analysis from Sanger sequencing (See FIG. 6A for comparison with amplicon sequencing). The MMR evasion strategy based on the installation of additional silent mutations and the epegRNAs used to target the ATP1A1 locus are illustrated. Point mutations are illustrated in orange. As described in FIG. 6, 2.5×10⁵ HSPCs were electroporated with PE2max RNAs targeting ATP1A1 and cultured in the presence or absence of 50 μM each dN and 25× in-house Vpx VLPs. Genomic DNA was harvested 3 days post-nucleofection. Data are from two out of three independent biological replicates performed with both sequencing methods. (FIG. 12B) Same as in (FIG. 12A), but HSPCs were electroporated with Vpx mRNA and PE2max RNAs targeting B2M (See FIG. 6B for comparison with amplicon sequencing). Data are plotted as mean±SEM from n=3 independent biological replicates performed with CD34⁺ HSPCs from three different donors. (FIG. 12C) PE and indel quantification as determined by BEAT and TIDE analysis from Sanger sequencing (See FIG. 7A for comparison with amplicon sequencing). As described in FIG. 7, 2.5×10⁵ HSPCs were electroporated with PE3/PE3bmax RNAs targeting HBB and cultured in the presence or absence of 5×Vpx VLPs (GPP) and 50 μM each dNs. Genomic DNA was harvested 3 days post-nucleofection. Data are plotted as mean±SEM from n=3 independent biological replicates performed with CD34⁺ HSPCs from one donor homozygous for the rs713040 allele (donor 2). Each biological replicate is illustrated with the same shade of grey. (FIG. 12D) Correlation of Sanger and amplicon sequencing quantifications at ATP141 (PE2max), B2M (PE2max), and HBB (PE3max and PE3bmax) for the 96 samples shown in (FIGS. 12B-12D). Figure discloses SEQ ID NOS 75-87, respectively, in order of appearance.

### DETAILED DESCRIPTION OF THE INVENTION

The present disclosure features methods for enhancing the efficiency of Reverse transcriptase (RT)-dependent gene editing systems and related compositions, and methods of using such systems for editing a polynucleotide (e.g., DNA, gene, genome).

Prime editing, Gene Writing, retrotransposition, and retrons are gene editing technologies that involve reverse transcription from an RNA template to synthesize DNA that is used to correct, replace, or insert (“write”) new genetic sequences into the genomes of living cells (e.g., hematopoietic stem and progenitor cells (HSPCs)). As detailed below, the invention is based, at least in part, on the discovery that methods of genome editing that involve reverse transcriptase are made more efficient by providing supplemental (i.e., exogenous) nucleotides directly to cells (e.g., hematopoietic stem and progenitor cells (HSPCs)) or indirectly by modulating nucleotide metabolism, for example, by inhibiting a cellular factor that degrades nucleotides, such as Sterile Alpha Motif Domain- and HD Domain-Containing Protein 1 (SAMHD1). In embodiments, VPX is used to inhibit SAMHD1.

The invention is also based, at least in part, on the discovery that deoxynucleoside supplementation and Vpx-mediated degradation of SAMHD1 improve prime editing efficiency in HSPCs, especially when coupled with editing approaches that evade mismatch repair. Therapeutic prime editing of hematopoietic stem and progenitor cells (HSPCs) is useful for treating or preventing blood disorders. Since quiescent cells (e.g., HSPCs) have low nucleotide levels and resist retroviral infection, modulation of nucleotide metabolism enhances the efficiency of reverse transcription mediated prime editing in HSPCs.

### Sterile Alpha Motif Domain- and HD Domain-Containing Protein 1 (SAMHD1)

SAMHD1 is a deoxynucleoside triphosphohydrolase that restricts the replication of lentiviruses in myeloid cells by hydrolyzing the cellular deoxynucleotide triphosphates to a level below that which is required for reverse transcription. Lentiviruses are retroviruses that include, for example, Human immunodeficiency virus type 2 (HIV-2), Simian Aids retrovirus SRV-1, and simian immunodeficiency viruses (SIVs). HIV-2 and some SIVs encode an accessory protein viral protein X (Vpx) that counteracts SAMHD1. Vpx recruits SAMHD1 to a cullin4A-RING E3 ubiquitin ligase (CRL4), which targets the enzyme for proteasomal degradation.

Accordingly, the disclosure provides for the use of VPX to enhance RT-dependent gene editing (e.g., Prime editing, gene writing, retrotransposition, and retrons) efficiency, alone or in combination with exogenous deoxynucleosides.

SAMHD1 expression markedly increases in hematopoietic stem and progenitor cells (HSPCs) after cytokine culture. Accordingly, the present disclosure also provides for the use of VPX to enhance RT-dependent gene editing (e.g., Prime editing, gene writing, retrotransposition, and retrons) efficiency, alone or in combination with exogenous deoxynucleosides, in HSPCs.

### Viral Protein X (VPX)

Viral Protein X (VPX) is an accessory gene found in HIV-2 and certain lineages of SIV. VPX is known to antagonize SAM domain and HD domain-containing protein 1 (SAMHD1) by inducing its ubiquitin-proteasome-dependent degradation. SAMHD1 is a gene that was found to restrict HIV-1 from infecting monocyte-derived macrophages (MDM) by hydrolyzing the cellular deoxynucleotide triphosphates (dNTP), reducing their level to below that required for the synthesis of the viral genomic DNA. As a result, VPX has been found to prevent the SAMHD1-mediated decrease in dNTP. In some embodiments, the systems and methods disclosed herein include a VPX polypeptide or polynucleotide encoding a VPX polypeptide. In some embodiments, the VPX polypeptide is an HIV-2 VPX polypeptide, an SIV VPX polypeptide, or variants or fragments thereof having activity in mediating the degradation of SAMHD1.

### Virus-Like Particles (VLPs)

Viral-like particles (VLPs) are nanoscale structures made up of assembled viral proteins that lack viral genetic material and are therefore non-infectious. VLPs are dispersed nanomaterials that can be produced in a variety of systems, including mammals, plants, insects, and bacteria. VLPs can be exploited as carriers for the delivery of bio- and nanomaterials, such as drugs, vaccines, quantum dots and imaging substances by virtue of the cavity within their structure. VLPs are made by the self-assembly of viral structural proteins. While VLPs are commonly produced using proteins(s) from a single virus type, chimeric VLPs can also be created by the assembly of structural proteins from different viruses.

Structural proteins from viruses, such as, but not limited to, lentivirus, gammaretroviruses (such as murine leukemia virus (MLV)), alpharetroviruses (such as Rous sarcoma virus (RSV)), human immunodeficiency virus (HIV), adeno-associated virus, Hepatitis B virus (HBV), Hepatitis C virus (HCV) and bacteriophages may be used to produce VLPs. In some embodiments, the VPX polypeptide is encapsulated by a VLP. In some embodiments, the VLP comprises structural proteins from lentivirus, gammaretroviruses, and/or alpharetroviruses.

VLPs may include envelope glycoproteins or other targeting proteins used to target the VLP to a specific cell type or to aid in membrane fusion. In some embodiments, the envelope glycoprotein or other targeting protein is VSV-G, BeEV-Rless (Bernadin O, Amirache F, Girard-Gagnepain A, Moirangthem R D, Lévy C, Ma K, Costa C, Nègre D, Reimann C, Fenard D, Cieslak A, Asnafi V, Sadek H, Mhaidly R, Cavazzana M, Lagresle-Peyrou C, Cosset F L, André I, Verhoeyen E. Baboon envelope LVs efficiently transduced human adult, fetal, and progenitor T cells and corrected SCID-X1 T-cell deficiency. Blood Adv. 2019 Feb. 12; 3 (3): 461-475; Girard-Gagnepain A, Amirache F, Costa C, Lévy C, Frecha C, Fusil F, Nègre D, Lavillette D, Cosset F L, Verhoeyen E. Baboon envelope pseudotyped LVs outperform VSV-G-LVs for gene transfer into early-cytokine-stimulated and resting HSCs. Blood. 2014 Aug. 21; 124 (8): 1221-31.), DSG2 (Wang H, Germond A, Li C, Gil S, Kim J, Kiem H P, Lieber A. In vivo HSC transduction in rhesus macaques with an HDAd5/3+ vector targeting desmoglein 2 and transiently overexpressing cxcr4. Blood Adv. 2022 Aug. 9; 6 (15): 4360-4372.), and/or scFV (Hamilton J R, Chen E, Perez B S, Sandoval Espinoza C R, Kang M H, Trinidad M, Doudna J A. Programmable enveloped delivery vehicles for human genome engineering in vivo [Internet]. bioRxiv. 2023 [cited 2023 Apr. 25]. p. 2022.08.24.505004. Available from: www.biorxiv.org/content/biorxiv/early/2023/04/02/2022.08.24.505004.).

### Prime Editing

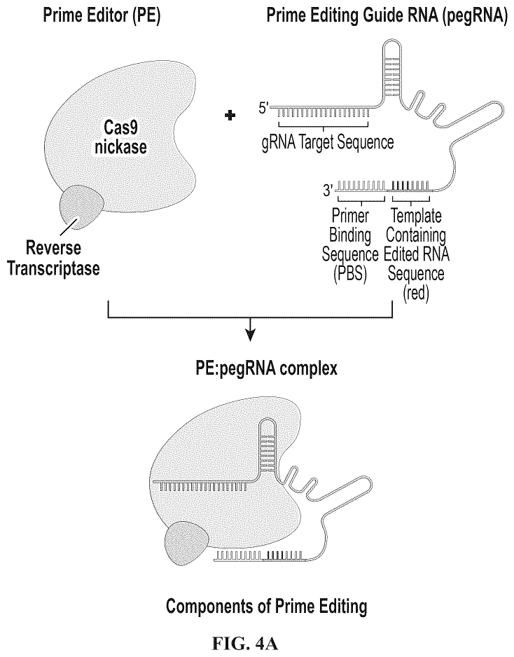

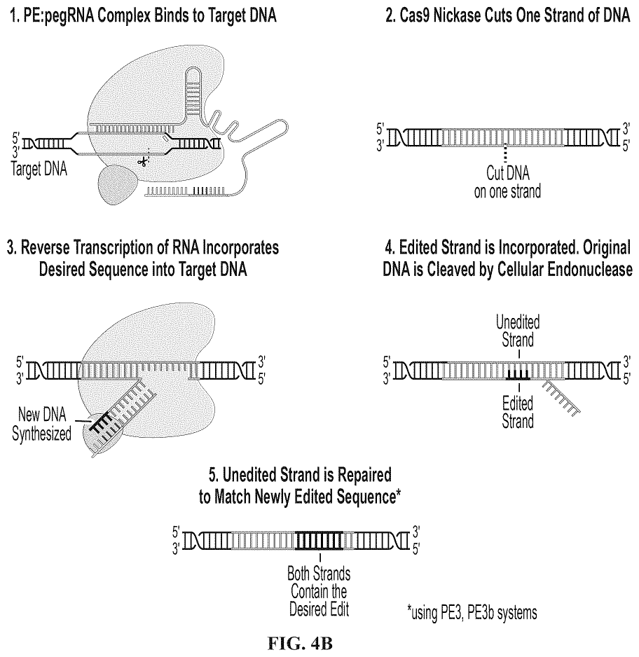

Prime editing is a gene editing method that can alter a target polynucleotide, for example, by targeted insertions, deletions, and base swapping in a precise way. Components of a CRISPR Prime Editing System (shown in FIG. 4A, https://www.synthego.com/guide/crispr-methods/prime-editing) include a prime editor, which comprises a Cas9 nickase that is fused to a reverse transcriptase, prime editing guide RNA, which includes a gRNA target sequence, primer binding sequence (PBS) and template containing edited RNA sequence in the context of a pegRNA having a particular tertiary structure (FIG. 4A).

While recent advances have improved the technology, efficient prime editing remains challenging particularly in primary hematopoietic cells including hematopoietic stem and progenitor cells (HSPCs)). One key difference between cancer cell lines and primary hematopoietic cells is the concentration of nucleotides available for reverse transcription. Nondividing cells typically have orders of magnitude lower nucleotide levels as compared to dividing cells.

### Genome Editing

Prime editing is just one form of gene editing. Therapeutic gene editing is a major focus of biomedical, biologic, and agricultural research, embracing the interface between basic and clinical science. A large number of different recessive hereditary human disease syndromes are caused by inheritance of biallelic inactivating point mutations of disease genes. The development of novel “gene editing” tools provides the ability to manipulate the DNA sequence of a cell at a specific chromosomal locus, without introducing mutations at other sites of the genome. This technology effectively enables the researcher to manipulate the genome of a subject's cells in vitro or in vivo, such as by effecting a reversion of a deleterious genotype, or adding protective or enhancing polynucleotide sequences.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule is introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. Importantly, if the donor DNA molecule differs slightly in sequence from the chromosomal sequence, HR-mediated DSB repair will introduce the donor sequence into the chromosome, resulting in gene conversion/gene correction of the chromosomal locus. In the context of therapeutic gene targeting, the altered sequence chosen would be an active or functional fragment (e.g., wild type, normal) of the disease gene of interest. By targeting the nuclease to a genomic site that contains the disease-causing point mutation, the concept is to use DSB formation to stimulate HR and to thereby replace the mutant disease sequence with wild-type sequence (gene correction). The advantage of the HR pathway is that it has the potential to generate seamlessly a wild-type copy of the gene in place of the previous mutant allele.

Current genome editing tools use the induction of double strand breaks (DSBs) to enhance gene manipulation of cells. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ.

Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ~18 bp sequences in the genome.

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339 (6121): 823-6). Unlike other gene therapy methods, which add a functional, or partially functional, copy of a gene to a patient's cells but retain the original dysfunctional copy of the gene, this system can remove the defect. Genetic correction using engineered nucleases has been demonstrated in tissue culture cells and rodent models of rare diseases.

CRISPR has been used in a wide range of organisms including bakers yeast (S. cerevisiae), zebra fish, nematodes (C. elegans), plants, mice, and several other organisms. Additionally CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (.about.30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337 (6096): 816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

gRNA

As an RNA guided protein, Cas9 utilizes a short RNA to direct the recognition of DNA targets. Though Cas9 preferentially interrogates DNA sequences containing a PAM sequence NGG it can bind here without a protospacer target. However, the Cas9-gRNA complex uses a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

In one approach, one or more cells of a subject are altered to express a wild-type form of a protein using a CRISPR-Cas system. Cas9 can be used to target a polynucleotide comprising a mutation. Upon target recognition, Cas9 induces double strand breaks in the target gene. Homology-directed repair (HDR) at the double-strand break site can allow insertion of a desired wild-type polynucleotide sequence.

The following US patents and patent publications relating to editing systems are incorporated herein by reference in their entirety: U.S. Pat. No. 8,697,359, 20140170753, 20140179006, 20140179770, 20140186843, 20140186958, 20140189896, 20140227787, 20140242664, 20140248702, 20140256046, 20140273230, 20140273233, 20140273234, 20140295556, 20140295557, 20140310830, 20140356956, 20140356959, 20140357530, 20150020223, 20150031132, 20150031133, 20150031134, 20150044191, 20150044192, 20150045546, 20150050699, 20150056705, 20150071898, 20150071899, 20150071903, 20150079681, 20150159172, 20150165054, 20150166980, and 20150184139.

### Prime Editing Process

Prime editing involves the use of a Cas endonuclease and a single guide (sg) RNA to edit sequences without generating a double-stranded break, prime editing uses Cas9 nickase—a variant of Cas9 that nicks the DNA rather than generating double-strand breaks—and a reverse transcriptase. This combination of a Cas9 and reverse transcriptase is referred to as a prime editor (PE). The reverse transcriptase may be untethered from the Cas9 nickase, as shown, for example, by Liu B et al. Nat Biotechnol. 2022 September; 40 (9): 1388-1393 and Grünewald J et al. Nat Biotechnol. 2023 March; 41 (3): 337-343.

At present, multiple versions of prime editors exist. PE1, which is the first version developed, is capable of generating insertions, deletions, and base transversions. PE2 contains certain modifications relative to PE1 that led to improved binding and thermostability. PE3 and PE3b include the ability to mend the mismatch sequences that occur with prime editing. See, for example, Huang et al., Front Bioeng Biotechnol. 2023; 11:1039315. PE3 installs another nick on the opposing strand as the nick to which the RT product 3′ flap is appended. PE3b is a subtype of PE3 in which the second nick can only occur after the initial steps of prime editing have occurred. The most recent versions, PE4 and PE5, are versions of PE2 and PE3 respectively in which DNA mismatch repair is inhibited, typically with dominant negative MLH1, to increase the efficiency of prime edit repairs. See, for example, Chen P J et al., Cell. 2021 Oct. 28; 184 (22): 5635-5652.e29. A variety of twin flap forms of prime editing repair also exist, such as twinPE and Prime-Del. See, for example, Anzalone A V et al., Nat Biotechnol. 2022 May; 40 (5): 731-740; Choi J et al., Nat Biotechnol. 2022 February; 40 (2): 218-226.

The guide RNA, called prime editing guide RNA (pegRNA), is substantially larger than standard sgRNAs commonly used for CRISPR gene editing (>100 nt vs. 20 nt). The pegRNA is a sgRNA with a primer binding sequence (PBS) and the template containing the desired RNA sequence added at the 3′ end. At present, pegRNAs are created using plasmids, using in-vitro transcription, and by chemical synthesis. Together, the prime editor and the pegRNA form a PE: pegRNA complex, which is used to mediate genome editing within a cell.

The prime editing process is shown in FIG. 4B (www.synthego.com/guide/crispr-methods/prime-editing). The PE:pegRNA complex binds to the target DNA, and Cas9 nicks only one strand, generating a flap. The Primer Binding Sequence, located on the pegRNA, binds to the DNA flap and the edited RNA sequence is reverse transcribed using the reverse transcriptase. The edited strand is incorporated into the DNA at the end of the nicked flap, and the target DNA is repaired with the new reverse transcribed DNA. The original DNA segment is removed by a cellular endonuclease. This leaves one strand edited, and one strand unedited. In newer PE systems, PE3 and PE3b, the unedited strand can be corrected to match the newly edited strand by using an additional standard guide RNA. In this case, the unedited strand is nicked by a Cas9 nickase and the newly edited strand is used as a template to repair the nick, thus completing the edit.

Prime editing is described, for example, in the following references: Anzalone A V, et al. Search-and-replace genome editing without double-strand breaks or donor DNA. Nature. 2019; Zhao D, Li J, Li S, Xin X, Hu M, Price M A, et al. Glycosylase base editors enable C-to-A and C-to-G base changes. Nat. Biotechnol. 2021; 39:35-40; Kurt I C, Zhou R, Iyer S, Garcia S P, Miller B R, Langner L M, et al. CRISPR C-to-G base editors for inducing targeted DNA transversions in human cells. Nat. Biotechnol. 2021; 39:41-6; Chen L, Park J E, Paa P, Rajakumar P D, Prekop H-T, Chew Y T, et al. Programmable C: G to G: C genome editing with CRISPR-Cas9-directed base excision repair proteins. Nat. Commun. 2021; 12:1384; and Liu Y, Li X, He S, Huang S, Li C, Chen Y, et al. Efficient generation of mouse models with the prime editing system. Cell Discov. 2020; 6:1-4.

### Prime Editor

The term “prime editor (PE)” refers to the polypeptide or polypeptide components involved in prime editing. A DNA binding domain that comprises a Cas9 nickase, a Cpf1 nickase, or another CRISPR-Cas nickase. In some embodiments, the DNA binding domain (e.g., a nucleic acid guided DNA binding domain) is a Cas protein domain. In some embodiments, the Cas protein is a Cas9; e.g., Cas9 nuclease; e.g., dCas9, Cas9 nickase.

In some embodiments, the CAS domain is fused to a reverse transcriptase domain (RT domain).

In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

In some embodiments, a prime editor comprises a Cas polypeptide (i.e., a DNA binding domain) and a reverse transcriptase polypeptide (i.e., a DNA polymerase domain) that are derived from different species. For example, a prime editor may comprise a S. pyogenes Cas9 polypeptide and a Moloney murine leukemia virus (M-MLV) reverse transcriptase polypeptide. In some embodiments, the prime editor comprises a fusion polypeptide that comprises a comprises a Cas polypeptide (i.e., a DNA binding domain) and a reverse transcriptase polypeptide (i.e., a DNA polymerase domain) that are derived from different species. For example, a prime editor may comprise a S. pyogenes Cas9 polypeptide and a Moloney murine leukemia virus (M-MLV) reverse transcriptase (RT) polypeptide.

### Prime Editing Guide RNAs (PEgRNAs)

The term “prime editing guide RNA”, or “PEgRNA”, refers to a guide polynucleotide that comprises one or more intended nucleotide edits for incorporation into a double stranded target polynucleotide, e.g., double stranded target DNA. In some embodiments, the PEgRNA associates with and directs a prime editor to incorporate the one or more intended nucleotide edits into the double stranded target DNA, e.g., a target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the double stranded target DNA, e.g., a target gene. Intended nucleotide edit may refer to the edit on the editing template as compared to the sequence on the target strand of the double stranded target DNA, e.g., a target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the double stranded target DNA, e.g., a target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the double stranded target DNA, e.g., a target gene, wherein the extended nucleotide sequence may be referred to as an extension arm.

In certain embodiments, the extension arm comprises a primer binding site sequence (PBS) that can initiate target-primed DNA synthesis. In some embodiments, the PBS is complementary or substantially complementary to a free 3′ end on the edit strand of the double stranded target DNA, e.g., a target gene at a nick site generated by the prime editor. In some embodiments, the extension arm further comprises an editing template that comprises one or more intended nucleotide edits to be incorporated in the double stranded target DNA, e.g., a target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the double stranded target DNA, e.g., a target gene. In some embodiments, the editing template comprises substantial or partial complementarity to the editing target sequence except at the position of the intended nucleotide edits to be incorporated into the double stranded target DNA, e.g., a target gene.

In some embodiments, a PEgRNA includes only RNA nucleotides and forms an RNA polynucleotide. In some embodiments, a PERNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PERNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PERNA, the 3′ portion of the PEgRNA, or in the middle of the gRNA core. In some embodiments, a PERNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5 ‘end of a spacer. In some embodiments, the gRNA core of a PEgRNA may be located at the 3’ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PERNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PERNA comprises, from 5′ to 3′: an editing target, a PBS, a spacer, and a gRNA core.

An intended nucleotide edit in an editing template of a PEgRNA may comprise various types of alterations as compared to the double stranded target DNA, e.g., a target gene sequence. In some embodiments, the nucleotide edit is a single nucleotide substitution as compared to the double stranded target DNA, e.g., a target gene sequence. In some embodiments, the nucleotide edit is a deletion as compared to the double stranded target DNA, e.g., a target gene sequence. In some embodiments, the nucleotide edit is an insertion as compared to the double stranded target DNA, e.g., a target gene sequence. In some embodiments, the editing template comprises one to ten intended nucleotide edits as compared to the double stranded target DNA, e.g., a target gene sequence. In some embodiments, the editing template comprises one or more intended nucleotide edits as compared to the double stranded target DNA, e.g., a target gene sequence. In some embodiments, the editing template comprises two or more intended nucleotide edits as compared to the double stranded target DNA, e.g., a target gene sequence. In some embodiments, the editing template comprises three or more intended nucleotide edits as compared to the double stranded target DNA, e.g., a target gene sequence. In some embodiments, the editing template comprises four or more, five or more, or six or more intended nucleotide edits as compared to the double stranded target DNA, e.g., a target gene sequence. In some embodiments, the editing template comprises two single nucleotide substitutions, insertions, deletions, or any combination thereof, as compared to the double stranded target DNA, e.g., a target gene sequence. In some embodiments, the editing template comprises three single nucleotide substitutions, insertions, deletions, or any combination thereof, as compared to the double stranded target DNA, e.g., a target gene sequence. In some embodiments, the editing template comprises four, five, or six single nucleotide substitutions, insertions, deletions, or any combination thereof, as compared to the double stranded target DNA, e.g., a target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PERNA may comprise one or more intended nucleotide edits, compared to the double stranded target DNA, e.g., a target gene, to be edited. Position of the intended nucleotide edit(s) relevant to other components of the PEgRNA, or to particular nucleotides (e.g., mutations) in the double stranded target DNA, e.g., a target gene, may vary. In some embodiments, the nucleotide edit is in a region of the PEgRNA corresponding to or homologous to the protospacer sequence. In some embodiments, the nucleotide edit is in a region of the PERNA corresponding to a region of the double stranded target DNA outside of the protospacer sequence.

In some embodiments, the position of a nucleotide edit incorporation in the double stranded target DNA, e.g., a target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3 ‘most nucleotide of the PAM sequence. In some embodiments, position of an intended nucleotide edit in the editing template may be referred to by aligning the editing template with the partially complementary edit strand of the double stranded target DNA, e.g., a target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5’ most nucleotide of the PAM sequence in the edit strand of the double stranded target DNA, e.g., a target gene. By 0 nucleotide upstream or downstream of a reference position, it is meant that the intended nucleotide is immediately upstream or downstream of the reference position. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the editing template is at a position corresponding to 6 nucleotides upstream of the 5 ‘most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5’ most nucleotide of the PAM sequence in the edit strand of the double stranded target DNA, e.g., a target gene. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence. In some embodiments, a nucleotide edit is incorporated at a position corresponding to 6 nucleotides downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to in the PERNA, positions of the one or more intended nucleotide edits may be referred to relevant to components of the PERNA. For example, an intended nucleotide edit may be 5′ or 3′ to the PBS. In some embodiments, a PERNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the double stranded target DNA, e.g., a target gene may also be referred to based on the nicking position generated by a prime editor based on sequence homology and complementarity. For example, in embodiments, the distance between the nucleotide edit to be incorporated into the double stranded target DNA, e.g., a target gene, and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5 ‘most position of the nucleotide edit and the 5’ most position of the PAM sequence.

In some embodiments, the editing template extends beyond a nucleotide edit to be incorporated to the double stranded target DNA, e.g., a target gene, sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 base pairs 3′ to the nucleotide edit to be incorporated to the double stranded target DNA, e.g., a target gene, sequence. In some embodiments, the editing template comprises at least 4 to 30 base pairs 3′ to the nucleotide edit to be incorporated to the double stranded target DNA, e.g., a target gene, sequence. In some embodiments, the editing template comprises at least 4 to 25 base pairs 3′ to the nucleotide edit to be incorporated to the double stranded target DNA, e.g., a target gene, sequence. In some embodiments, the editing template comprises at least 4 to 20 base pairs 3′ to the nucleotide edit to be incorporated to the double stranded target DNA, e.g., a target gene, sequence. In some embodiments, the editing template comprises at least 4 to 30 base pairs 5′ to the nucleotide edit to be incorporated to the double stranded target DNA, e.g., a target gene, sequence. In some embodiments, the editing template comprises at least 4 to 25 base pairs 5′ to the nucleotide edit to be incorporated to the double stranded target DNA, e.g., a target gene, sequence. In some embodiments, the editing template comprises at least 4 to 20 base pairs 5′ to the nucleotide edit to be incorporated to the double stranded target DNA, e.g., a target gene, sequence.

The editing template of a PEgRNA may encode a new single stranded DNA (e.g. by reverse transcription) to replace a target sequence in the double stranded target DNA, e.g., a target gene. In some embodiments, the editing target sequence in the edit strand of the double stranded target DNA, e.g., a target gene is replaced by the newly synthesized strand, and the nucleotide edit(s) are incorporated in the region of the double stranded target DNA, e.g., a target gene. In some embodiments, the newly synthesized DNA strand replaces the editing target sequence in the double stranded target DNA, e.g., a target gene, wherein the editing target sequence (or the endogenous sequence complementary to the editing target sequence on the target strand of the target gene) comprises a mutation compared to a wild-type sequence of the same gene, wherein incorporation of the one or more intended nucleotide edits corrects the mutation.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may use different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Casl2b-based prime editor.

### Engineered Retrons

Compositions described herein are also useful in enhancing the activity of the ret gene product, which has reverse transcriptase activity. Retron elements may be about 2 kb long. They contain a single operon controlling the synthesis of an RNA transcript carrying three loci, msr, msd, and ret, that are involved in msDNA synthesis. The retron operon carries a promoter sequence P that controls the synthesis of an RNA transcript carrying the three loci, msr, msd, and ret. The ret gene product, a reverse transcriptase, processes the msd/msr portion of the RNA transcript (non coding RNA or “ncRNA”) into msDNA. Accordingly, the DNA portion of msDNA is encoded by the msd gene, the RNA portion is encoded by the msr gene, while the product of the ret gene is a reverse transcriptase similar to the RTs produced by retroviruses and other types of retroelements. The ret gene product is responsible for processing the msd/msr portion of the RNA transcript (ncRNA) into msDNA.

Internal base pairing creates various stem-loop/hairpin secondary structures in the msDNA. The retron ncRNA folds into a characteristic secondary structure that contains a conserved guanosine residue at the end of a stem loop. Synthesis of DNA by the retron-encoded reverse transcriptase (RT) results in the DNA/RNA chimera which is composed of small single-stranded DNA linked to small single-stranded RNA. The RNA strand is joined to the 5′ end of the DNA chain via a 2′-5′ phosphodiester linkage that occurs from the 2′ position of the conserved internal guanosine residue. The RT recognizes this secondary structure and uses a conserved guanosine residue in the msr as a priming site to reverse transcribe the msd sequence and produce a hybrid ssRNA-ssDNA molecule referred to as msDNA. The 5′ and 3′ ends of ncRNA are referred to generally as the a1 and a2 complementary regions and can hybridize to one another to form a stem or duplex region referred to as the “a1/a2 stem” or the “a1/a2 duplex” of the ncRNA. The use of retrons in CRISPR gene editing arrays is described, for example, in U.S. patent application Ser. No. 16/499,889, which is incorporated herein by reference in its entirety.

In certain embodiments, sequences of the msr gene, msd gene, and ret gene used in the engineered retron may be derived from a bacterial retron operon. Representative retrons are available such as those from gram-negative bacteria including, without limitation, myxobacteria retrons such as Myxococcus xanthus retrons (e.g., Mx65, Mx162) and Stigmatella auwntiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81, Vc95, Vc137); Vibrio parahaemolyticus (e.g., Vc96); and Nannocysids eredens retrons (e.g., Ne144). Retron msr gene, msd gene, and ret gene nucleic acid sequences as well as retron reverse transcriptase protein sequences may be derived from any source. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties. Any of these retron sequences or a variant thereof comprising a sequence can include variant nucleotides, added nucleotides, or fewer nucleotides. For example, the retrons can have at least about 80-100/6 sequence identity thereto, including any percent identity within this range, such as 81, 82, 83, 84, 85, 86, 87, 88, 89, 90, 91, 92, 93, 94, 95, 96, 97, 98, or 99% sequence identity to any of the retron sequences described herein (including those defined by accession number), and can be used to construct an engineered retron or vector system comprising an engineered retron, as described herein.

In some embodiments, the retron construct is modified with respect to the native retron to include a heterologous sequence of interest. In this context, the retrons can be engineered with heterologous sequences for use in a variety of applications. For example, heterologous sequences can be added to retron constructs to provide a cell with a nucleic acid encoding a protein or regulatory RNA of interest, a donor polynucleotide suitable for use in gene editing, e.g., by homology directed repair (HDR) or recombination-mediated genetic engineering (recombineering). Such heterologous sequences may be inserted, for example, into the msr gene or the msd gene such that the heterologous sequence is transcribed by the retron reverse transcriptase as part of the msDNA product. The ret gene product is responsible for processing the msd/msr portion of the RNA transcript into msDNA.

In some embodiments, programmable nucleases (e.g., CRISPR nucleases, proteins containing zinc finger domains (ZFP), or proteins containing TALE domains (e.g., TALEN)) combined with retrons for use as gene editors. In some embodiments, the retrons include (1) a guide RNA (e.g., a CRISPR crRNA-tracrRNA) linked or inserted into an a1 or a2 complementary region of the ncRNA; and (2) a heterologous sequence inserted into the ncRNA msd gene. In some embodiments, the heterologous sequence can comprise a single strand DNA product of reverse transcription which comprises a nucleotide sequence having a sequence modification (e.g., a desired one or more mutations, insertion, deletion, or inversion) that is flanked by regions of homology to a target genomic site. Retron-based genome editing systems are known in the art and described, for example, in US20190330619A1, PCT/US2022/079220, each of which is incorporated herein by reference in their entirety.

### Retrotransposons

Retrotransposons can contain transposable elements that are active participants in reorganizing their resident genomes. Broadly, retrotransposons can refer to DNA sequences that are transcribed into RNA and translated into protein and have the ability to reverse-transcribe themselves back into DNA. Approximately 45% of the human genome is comprised of sequences that result from transposition events. Retrotransposition occasionally generates target site deletions or adds non-retrotransposon DNA to the genome by processes termed 5′- and 3′-transduction. Recombination between non-homologous retrotransposons causes deletions, duplications or rearrangements of gene sequence. Ongoing retrotransposition can generate novel splice sites, polyadenylation signals and promoters, and so builds new transcription modules. The use of retrotransposons in methods of genomic integration may be found, for example, in U.S. patent application Ser. No. 17/687,395.

The systems herein may comprise one or more “CRISPR-associated transposases” (also used interchangeably with Cas-associated transposases, CRISPR-associated transposase proteins, or CAST system herein) or functional fragments thereof. CRISPR-associated transposases may include any transposases or transposase subunity that can be directed to or recruited to a region of a target polynucleotide by sequence-specific binding of a CRISPR-Cas complex to the target polynucleotide. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g. by a linker) to one or more components in a CRISPR-Cas system, e.g., Cas protein, guide molecule etc.). In embodiments, the term transposase refers to an enzyme, which is a component of a functional nucleic acid-protein complex capable of transposition and which mediates transposition.

Generally, retrotransposons may be grouped into two classes, the retrovirus-like long terminal repeat (LTR) retrotransposons, and the non-LTR elements such as human LINE-1 elements, Neurospora TAD elements (Kinsey, 1990, Genetics 126:317-326), I factors from Drosophila (Bucheton et al., 1984, Cell 38:153-163), and R2Bm from Bombyx mori (Luan et al., 1993, Cell 72:595-605). These two types of retrotransposons are structurally different and also retrotranspose using radically different mechanisms.

Human LINE-1 elements are capable of retrotransposition in human cells as well as cells of other animal species and can be manipulated in a versatile manner to achieve efficient delivery and integration of a genetic cargo into the genome of a cell. Such LINE-1 elements have a variety of uses in human and animal genetics including, but not limited to, uses in diagnosis and treatment of genetic disorders and in cancer. The LINE-1 elements of the disclosure are also useful for the treatment of various phenotypic effects of various diseases. For example, LINE-1 elements may be used for transfer of DNA encoding anti-tumorigenic gene products into cancer cells. Other uses of the LINE-1 elements of the disclosure will become apparent to the skilled artisan upon a reading of the present specification.

In general, a human LINE-1 element comprises a 5′ UTR with an internal promoter, two non-overlapping reading frames (ORF1 and ORF2), a 200 bp 3′ UTR and a 3′ poly A tail. The LINE-1 retrotransposon can also comprise an endonuclease domain at the LINE-1 ORF2 N-terminus. The finding that LINE-1 encodes an endonuclease demonstrates that the element is capable of autonomous retrotransposition. LINE-1 is a modular protein that contains non-overlapping functional domains which mediate its reverse transcription and integration. In some embodiments, the sequence specificity of the LINE-1 endonuclease itself can be altered or the LINE-1 endonuclease can be replaced with another site-specific endonuclease.

The LINE-1 retrotransposon may be manipulated using recombinant DNA technology to comprise and/or be contiguous with, other DNA elements which render the retrotransposon suitable for insertion of substantial lengths (up to 1 kb, or greater than 1 kb) of heterologous or homologous DNA into the genome of a cell. The LINE-1 retrotransposon may also be manipulated using the same type of technology such that insertion of the DNA into the genome of a cell is site-directed (site into which such DNA is inserted is known). Alternatively, the LINE-1 retrotransposon may be manipulated such that the insertion site of the DNA is random. The retrotransposon may also be manipulated to effect insertion of a desired DNA sequence into regions of DNA which are normally transcriptionally silent, wherein the DNA sequence is expressed in a manner such that it does not disrupt the normal expression of genes in the cell. In some embodiments, the integration or retrotransposition is in the trans orientation. In some embodiments, the integration or retrotransposition occurs in the cis orientation.

Exemplary, non-limiting examples of LINE-encoded polypeptides are found in GenBank Accession Nos. AAC51261, AAC51262, AAC51263, AAC51264, AAC51265, AAC51266, AAC51267, AAC51268, AAC51269, AAC51270, AAC51271, AAC51272, AAC51273, AAC51274, AAC51275, AAC51276, AAC51277, AAC51278 and AAC51279.

The LINE-1 retrotransposon expresses a 6-kb bicistronic RNA that encodes the 40 kDa Open Reading Frame-I RNA-binding protein (ORFlp) of essential but uncertain function, and a 150 kDa ORF2 protein with endonuclease and reverse transcriptase (RT) activities. LINE-1 retrotransposition is a complex process involving transcription of the LINE-1, transport of its RNA to the cytoplasm, translation of the bicistronic RNA, formation of a ribonucleoprotein (RNP) particle, its re-import to the nucleus and target-primed reverse transcription at the integration site. A few transcription factors that interact with LINE-1 s have been identified. Transcribed LINE-1 RNA forms an RNP in cis with the proteins that are translated from the transcript. Without intending to be bound by theory, LINE-I integrates into genomic DNA by target-site primer reverse transcription (TPRT) by ORF2p cleavage at the 5′-TTTT-3′ where a poly A sequence of LINE-I RNA anneals and primes reverse transcriptase (RT) activity to make LINE-1 cDNA.

The process of TPRT, which is unique to non-LTR retrotransposons, is initiated by the targeting endonuclease that cleaves one (bottom) strand of the target site and creates a free 3′-hydroxyl end, which is used as a primer for reverse transcription. Thus, and without intending to be bound by theory, target-site selection of sequence-specific non-LTR retrotransposons may be determined by the endonuclease domain itself.

Many of the RLE-encoding non-LTR retrotransposons are also site-specific. All clades of RLE-encoding non-LTR elements (R2, R4, NeSL, CRE, and HERO) encode some DNA-binding motifs, one to three zinc fingers (ZFs) at the N-terminal end and one CCHC (cysteine-histidine) motif C-terminal to the RT domain within a multifunctional single ORF. R2-clade elements also encode a Myb-like domain. In contrast to APE-retrotransposons, the functional roles of RLE-type endonuclease itself in sequence specificity is not known clearly to date, and the target-site recognition is achieved primarily through the DNA-binding motifs mentioned above.

Accordingly, the disclosure provides for retrotransposon-base gene editors including an RNA which encodes a heterologous sequence, a sequence encoding a reverse transcriptase, and a sequence encoding an endonuclease. In some embodiments, sequence specificity of the target site may be determined either by the endonuclease or by DNA binding motifs included in the RNA sequence. In some embodiments, the same sequence may encode both the endonuclease and reverse transcriptase.

In some embodiments, the endonuclease is a mega-TAL nuclease domain, TALENS, or a zinc finger nuclease domain, for example, a mega-TAL, a TALE, or a zinc finger domain fused to or associated with a nuclease domain, e.g., a FokI nuclease domain. In some embodiments, the endonuclease is a CRISPR-Cas protein domain loaded with a specific guide nucleic acid, e.g., a guide RNA (gRNA) for a specific target locus. In some embodiments, the CRISPR-Cas protein is a Cas9, a Cas12a, a Cas12b, a Casi 3, a CasX, or a CasY protein domain.

The use of retrotransposons for targeted gene modification is described, for example, in PCT/US2020/061114, and in U.S. Pat. No. 11,384,344, each of which is incorporated herein by reference in its entirety.

### Gene Writer

Non-long terminal repeat (LTR) retrotransposons are a type of mobile genetic elements that are widespread in eukaryotic genomes. They include two classes: the apurinic/apyrimidinic endonuclease (APE)-type and the restriction enzyme-like endonuclease (RLE)-type. The APE class retrotransposons are comprised of two functional domains: an endonuclease/DNA binding domain, and a reverse transcriptase domain. The RLE class are comprised of three functional domains: a DNA binding domain, a reverse transcription domain, and an endonuclease domain. The reverse transcriptase domain of non-LTR retrotransposon functions by binding an RNA sequence template and reverse transcribing it into the host genome's target DNA. The RNA sequence template has a 3′ untranslated region which is specifically bound to the transposase, and a variable 5′ region generally having Open Reading Frame(s) (“ORF”) encoding transposase proteins. The RNA sequence template may also comprise a 5′ untranslated region which specifically binds the retrotransposase.

The elements of such non-LTR retrotransposons can be functionally modularized and/or modified to target, edit, modify or manipulate a target DNA sequence, e.g., to insert an object (e.g., heterologous) nucleic acid sequence into a target genome, e.g., a mammalian genome, by reverse transcription. Such modularized and modified nucleic acids, polypeptide compositions and systems are referred to as Gene Writer™ gene editing systems. A Gene Writer™ gene editing system comprises: (A) a polypeptide or a nucleic acid encoding a polypeptide, wherein the polypeptide comprises (i) a reverse transcriptase domain, and either (x) an endonuclease domain that contains DNA binding functionality or (y) an endonuclease domain and separate DNA binding domain; and (B) a template RNA comprising (i) a sequence that binds the polypeptide and (ii) a heterologous insert sequence. For example, the Gene Writer genome editor protein may comprise a DNA-binding domain, a reverse transcriptase domain, and an endonuclease domain. In other embodiments, the Gene Writer genome editor protein may comprise a reverse transcriptase domain and an endonuclease domain. In certain embodiments, the elements of the Gene Writer™ gene editor polypeptide can be derived from sequences of non-LTR retrotransposons, e.g., APE-type or RLE-type retrotransposons or portions or domains thereof. In some embodiments the RLE-type non-LTR retrotransposon is from the R2, NeSL, HERO, R4, or CRE clade. In some embodiments the Gene Writer genome editor is derived from R4 element X4 Line, which is found in the human genome. In some embodiments the APE-type non-LTR retrotransposon is from the R1, or Tx1 clade. In some embodiments the Gene Writer genome editor is derived from Tx1 element Mare6, which is found in the human genome. The RNA template element of a Gene Writer™ gene editor system is typically heterologous to the polypeptide element and provides an object sequence to be inserted (reverse transcribed) into the host genome. In some embodiments the Gene Writer genome editor protein is capable of target primed reverse transcription. The use of Gene Writer systems in methods of genetic editing may be found, for example, in U.S. patent application Ser. No. 16/706,448.

In some embodiments the Gene Writer genome editor is combined with a second polypeptide. In some embodiments the second polypeptide is derived from an APE-type non-LTR retrotransposon. In some embodiments the second polypeptide has a zinc knuckle-like motif. In some embodiments the second polypeptide is a homolog of Gag proteins.

### Polypeptide Component of Gene Writer Gene Editor System

The reverse transcriptase domain of the Gene Writer system may be based on a reverse transcriptase domain of an APE-type or RLE-type non-LTR retrotransposon. A wild-type reverse transcriptase domain of an APE-type or RLE-type non-LTR retrotransposon can be used in a Gene Writer system or can be modified (e.g., by insertion, deletion, or substitution of one or more residues) to alter the reverse transcriptase activity for target DNA sequences. In some embodiments the reverse transcriptase is altered from its natural sequence to have altered codon usage, e.g. improved for human cells. In some embodiments the reverse transcriptase domain is a heterologous reverse transcriptase from a different retrovirus, LTR-retrotransposon, or non-LTR retrotransposon. In certain embodiments, a Gene Writer system includes a polypeptide that comprises a reverse transcriptase domain of an RLE-type non-LTR retrotransposon from the R2, NeSL, HERO, R4, or CRE clade, or of an APE-type non-LTR retrotransposon from the R1, or Tx1 clade.

In some embodiments, the endonuclease/DNA binding domain of an APE-type retrotransposon or the endonuclease domain of an RLE-type retrotransposon can be used or can be modified (e.g., by insertion, deletion, or substitution of one or more residues) in a Gene Writer system.

In some embodiments, the DNA-binding domain can be any one or more of Cas9, TAL domain, ZF domain, Myb domain, combinations thereof, or multiples thereof.

### Pharmaceutical Compositions

Compositions comprising one or more RT-dependent gene editing systems (e.g., prime editing), VPX polypeptides or polynucleotides encoding VPX polypeptides, or variants or fragments thereof having activity in mediating the degradation of SAMHD1, and/or exogenous deoxynucleotides or deoxynucleosides, as described herein are provided. In some embodiments, the compositions further comprise a pharmaceutically acceptable carrier, diluent, excipient, or vehicle.

Compositions and preparations (e.g., physiologically or pharmaceutically acceptable compositions) containing RT-dependent gene editing systems (e.g., prime editing system), VPX polypeptides or polynucleotides encoding VPX polypeptides, or variants or fragments thereof having activity in mediating the degradation of SAMHD1, and/or deoxynucleotides or deoxynucleosides for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of an RT-dependent gene editing systems (e.g., prime editing system), VPX polypeptides or polynucleotides encoding VPX polypeptides, or variants or fragments thereof having activity in mediating the degradation of SAMHD1, and/or deoxynucleotides or deoxynucleosides, as described herein, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

### Methods of Treatment

Methods of treating a disease (e.g., any disease treatable by correction of a pathogenic human gene variant by a RT-dependent gene editing system (e.g., prime editing system), or symptoms thereof, are provided. In some embodiments, genome editing with an RT-dependent gene editing system is carried out on a cell in vitro or in vivo. In other embodiments, methods of treating a disease by genome editing is carried out, for example, using retrons, engineered retrons, retrotransposons, or Gene Writing™ together with other components useful for introducing an alteration in the genome of a cell in the presence of i) a VPX polypeptide or a polynucleotide encoding said polypeptide, where the VPX polypeptide is capable of degrading SAMHD1, and/or ii) exogenously added deoxynucleotides or deoxynucleosides.

In one embodiment, a cell (e.g., HSC or HSPC) of a subject (e.g., a subject having or at risk of developing a genetic disorder) is contacted with a Prime Editing System, which comprises a Prime Editor and a pegRNA, in the presence of i) a VPX polypeptide or a polynucleotide encoding said polypeptide, where the VPX polypeptide is capable of degrading SAMHD1, and/or ii) exogenously added deoxynucleotides or deoxynucleosides. In some embodiments, the cell is contacted with the Prime Editing System in vitro. Once the edits have been carried out on the cell in vitro, the cell or a pharmaceutical composition comprising the cell is administered to the subject. Such administration may be by local injection or by system administration (e.g., by infusion).

In some embodiments, the cell is a T cell. In some embodiments, the genome editing includes installation of gain-of-function mutations which increase the antitumor activity of T lymphocytes (e.g., CAR-T cells, tumor-infiltrating lymphocytes, etc), which may then be used, for example, in the treatment of neoplasia (e.g., cancer immunotherapy applications). Examples of gain-of-function mutations which increase the antitumor activity of T lymphocytes include, but are not limited to, disruption of T cell receptor chains to prevent graft-versus-host disease (Eyquem J et al., Nature. 2017 Mar. 2; 543 (7643): 113-117; Ottaviano G et al., Sci Transl Med. 2022 Oct. 26; 14 (668): eabq3010), mutations to the immunosuppressive checkpoint PD-1 (Stadtmauer E A et al., Science. 2020 Feb. 28; 367 (6481): eaba7365), mutations to the adenosine A2a receptor (Giuffrida L et al., Nat Commun. 2021 May 28; 12 (1): 3236), mutations to REGNASE-1 (Wei J et al., Nature. 2019 December; 576 (7787): 471-476), and mutations to RASA2 (Carnevale J et al., Nature. 2022 September; 609 (7925): 174-182). In some embodiments, the cell is a CAR-T cell (e.g., a non-activated potent CAR-T cell). In some embodiments, the methods include facilitation of the production of CAR-T cells, for example, such as with lentiviral transduction in a media supplemented with 50 μM dNs (Ghassemi S et al., Nat Biomed Eng. 2022 February; 6 (2): 118-128). Without intending to be bound by theory, the present disclosure includes methods which facilitate the efficient installation of precise therapeutic edits in resting T cells, thereby facilitating the manufacture of CAR-T cells.

In other embodiments, a cell of the subject is contacted in vivo with a Prime Editing System in the presence of i) a VPX polypeptide or a polynucleotide encoding said polypeptide, where the VPX polypeptide is capable of degrading SAMHD1, and/or ii) exogenously added deoxynucleotides or deoxynucleosides.

The disclosure provides methods of treating a subject suffering from, or at risk of, or susceptible to disease, or a symptom thereof, or delaying the progression of a disease. In some embodiments, the method includes administering to the subject (e.g., a mammalian subject), a therapeutic amount of a cell that has been edited according to the methods described herein. In other embodiments, where editing is to take place in vivo, a Prime Editing System is used to contact a cell of the subject in vivo together with VPX polypeptides or polynucleotides encoding VPX polypeptides, or variants or fragments thereof having activity in mediating the degradation of SAMHD1, and/or exogenously added deoxynucleotides or deoxynucleosides, as described herein.

In some embodiments, the methods herein include administering to the subject (including a human subject identified as in need of such treatment) an effective amount of an RT-dependent gene editing system (e.g., Prime Editing System, retron, retrotransposase), VPX polypeptides or polynucleotides encoding VPX polypeptides, or variants or fragments thereof having activity in mediating the degradation of SAMHD1, and/or exogenously added deoxynucleotides or deoxynucleosides. The treatment methods are suitably administered to subjects, particularly humans, suffering from, are susceptible to, or at risk of having a disease, or symptoms thereof, namely, any disease treatable by correction of a pathogenic human gene variant by an RT-dependent gene editing system. Nonlimiting examples of diseases treatable by correction of a pathogenic gene variant by an RT-dependent gene editing system include, but are not limited to, hemoglobinopathies (e.g., sickle cell disease, beta-thalassemia, alpha-thalassemia), enzymopathies (e.g., pyruvate kinase deficiency), membranopathies (e.g., hereditary spherocytosis), inherited bone marrow-failure disorders (e.g., Fanconi anemia, dyskeratosis congenita, severe congenital neutropenia, Diamond-Blackfan anemia, congenital amegakaryocytic thrombocytopenia), disorders of leukocyte function (e.g., chronic granulomatous disease), inherited immunity disorders (e.g., severe combined immunodeficiency), disorders of the complement system (e.g., atypical hemolytic uremic syndrome), inherited metabolic disorders (e.g., phenylketonuria), inherited lung diseases (e.g., cystic fibrosis), inherited liver disorders (e.g., hereditary tyrosinemia), bleeding disorders (e.g., hemophilia), chronic infectious diseases (e.g., HIV infection), inherited neurological conditions (e.g., Huntington's disease), lysosomal storage disorders (e.g., Mucopolysaccharidosis Type 1), disorders where gremlin or somatic mutations are associated with disease, disorders in which genetic modification could achieve a therapeutic effect, or any disease treatable by correction of a pathogenic gene variant by an RT-dependent gene editing technology, including those variants listed in the Clin Var database (www.ncbi.nlm.nih.gov/clinvar/). Exemplary diseases and methods of treating such diseases using gene editing methods may be found in Eyquem J et al. Nature. 2017 Mar. 2; 543 (7643): 113-117.

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. A subject undergoing treatment can be a non-human mammal, such as a veterinary subject, or a human subject (also referred to as a “patient”).

In addition, prophylactic methods of preventing or protecting against a disease (e.g., any disease treatable by correction of a pathogenic human gene variant by an RT-dependent gene editing system), or symptoms thereof, are provided. Such methods comprise administering a cell edited using an RT-dependent Editing System (e.g., Prime Editing System) or a therapeutically effective amount of a pharmaceutical composition comprising an RT-dependent Gene Editing System (e.g., Prime Editing System), VPX polypeptides or polynucleotides encoding VPX polypeptides, or variants or fragments thereof having activity in mediating the degradation of SAMHD1, and/or deoxynucleotides or deoxynucleosides, as described herein to a subject (e.g., a mammal, such as a human), in particular, prior to development or onset of a disease. In some embodiments, such prophylactic methods of preventing or protecting against a disease include installation of protective genetic variants for acquired or inherited diseases, such as, but not limited to, CCR5-delta32 mutation for HIV infection, reducing PCSK9 expression for hypercholesterolemia, or hereditary persistence of fetal hemoglobin mutations for beta-hemoglobinopathies.

### Methods of Delivery

A cell edited in vitro using an RT-dependent Gene Editing System (e.g., Prime Editor System) can be administered to a subject by any of the routes normally used for introducing a cell to a subject. Where in vivo editing of cells is contemplated, an RT-dependent Gene Editing System (e.g., Prime Editor System) can be administered to a subject by any of the routes normally used for introducing a recombinant polypeptide. Routes and methods of administration include, without limitation, parenteral, such as intravenous (IV), intradermal, intramuscular, intraperitoneal, intrathecal, or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides, can be administered in any suitable manner, such as with pharmaceutically acceptable carriers, diluents, or excipients as described supra. Pharmaceutically acceptable carriers are determined in part by the particular composition being administered, as well as by the particular method used to administer the composition. Accordingly, a pharmaceutical composition comprising the edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides, can be prepared using a wide variety of suitable and physiologically and pharmaceutically acceptable formulations. In some embodiments, the disclosed methods include contacting a target DNA sequence (e.g., a pathogenic human gene variant) with an RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides.

Administration of the edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides, or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., any disease treatable by correction of a pathogenic human gene variant). The dose required will vary from subject to subject depending on the species, age, weight and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides, or pharmaceutical compositions thereof, that provide a therapeutic effect or protection against disease (e.g., any disease treatable by correction of a pathogenic human gene variant) suitable for administering to a subject in need of treatment or protection.

In some embodiments, edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosidesor a pharmaceutical composition thereof, is administered as a maximum-tolerated dose (MTD). In some embodiments, MTD is the dose with estimated probability of dose limiting toxicity (DLT) closest to the target toxicity rate of 20%. In some embodiments, edited cell or the RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides or a pharmaceutical composition thereof, is administered in a therapeutically effective dose for a mammal (e.g., human). In some embodiments, the mammal is a mouse. In some embodiments, the mammal is a human.

### Kits

Also provided are kits containing an RT-dependent Gene Editing System (e.g., Prime Editor System), VPX polypeptides or polynucleotides encoding VPX polypeptides, or variants or fragments thereof having activity in mediating the degradation of SAMHD1, and/or exogenous deoxynucleotides or deoxynucleosides, VPX polypeptides or polynucleotides encoding VPX polypeptides, or variants or fragments thereof having activity in mediating the degradation of SAMHD1, and/or deoxynucleotides or deoxynucleosides, or a pharmaceutically acceptable composition thereof, as described herein, and a pharmaceutically acceptable carrier, diluent, or excipient, for administering to a subject, for example. In some embodiments, the kit is provided for treating any disease treatable by correcting a pathogenic gene variant in a subject (e.g., human). In some embodiments, the kit will contain one or more of an RT-dependent gene editing system and pegRNAs, or vectors or other polynucleotides encoding such editing systems and pegRNAs, VPX polypeptides or polynucleotides encoding VPX polypeptides, or variants or fragments thereof having activity in mediating the degradation of SAMHD1, and/or deoxynucleotides or deoxynucleosides, as disclosed herein. The VPX may be in the form of a polypeptide or a polynucleotide encoding VPX, as described herein. In some embodiments, the kit comprises a vector containing a nucleotide sequence encoding VPX as disclosed herein. As will be appreciated by the skilled practitioner in the art, such a kit may contain one or more containers, labels, carriers, diluents or excipients, as necessary, and instructions for use.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: VPX Virus-Like Particles (VLPs) Enhanced Prime Editing in Human Hematopoietic Stem and Progenitor Cells (HSPCs)

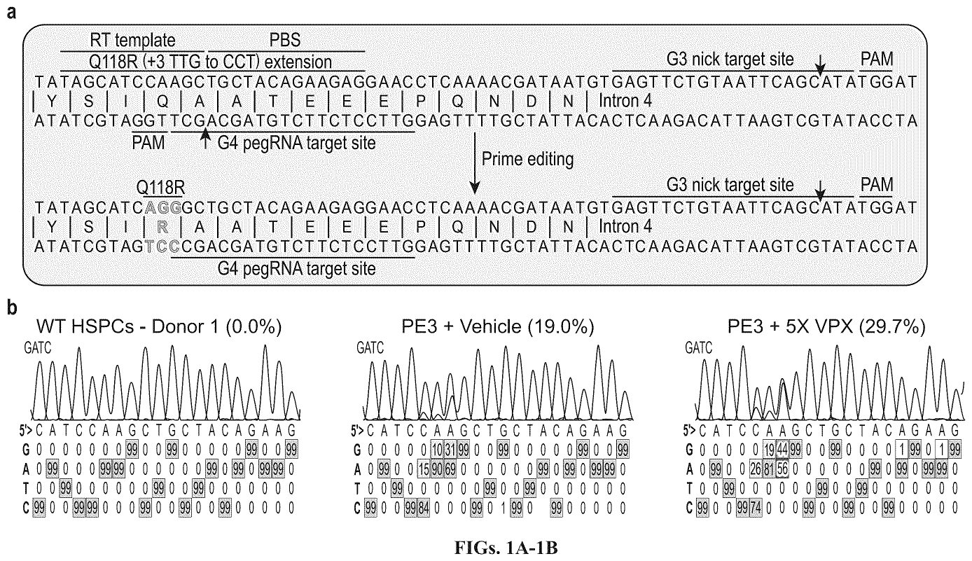

To determine whether VPX virus-like particles could enhance prime editing the following experiment was conducted. Prime editing was carried out on hematopoietic stem cells and progenitors thereof to generate a Q118R at ATP1A1 in the presence or absence of VPX VLPs. The editing was carried out as follows. Hematopoietic stem and progenitor cells (HSPCs) were thawed and cultured for 24 hours before electroporation. 5×10⁵ HSPCs were electroporated with 2000 ng Prime Editor 2 (PE2) mRNA+200 pmol of a prime editing RNA designed to introduce the Q118R mutation, synthetic ATPIA1-Q118R epegRNA+100 pmol synthetic nick sgRNA using pulse code DS-130. HSPCs were then cultured for 72 hours in the presence or absence of 5×VPX VLPs. 1× lentiBOOST™ was used in all conditions. DMEM supplemented with 10% FBS and 1% Pen/Strep was used as a vehicle control for VPX virus-like particles (VLPs). Genomic DNA was harvested 3 days post-nucleofection and sequenced to detect the presence or absence of edits. As expected, a Q118R mutation was installed at ATP1A1 using the prime editing system 3 (PE3). Interestingly, when prime editing was carried out in the presence of VPX, an increase in prime editing efficiency was observed (FIG. 1B).

### Example 2: VPX Virus-Like Particles (VLPs) and the Addition of Deoxynucleosides (dNs) Enhanced Prime Editing in HSPCs

To determine whether VPX virus-like particles and exogenously added deoxynucleosides could enhance prime editing the following experiment was conducted. Prime editing was carried out on hematopoietic stem cells and progenitors thereof to generate a Q118R at ATP141 in the presence or absence of VPX VLPs and exogenous deoxynucleosides. The editing was carried out as follows.

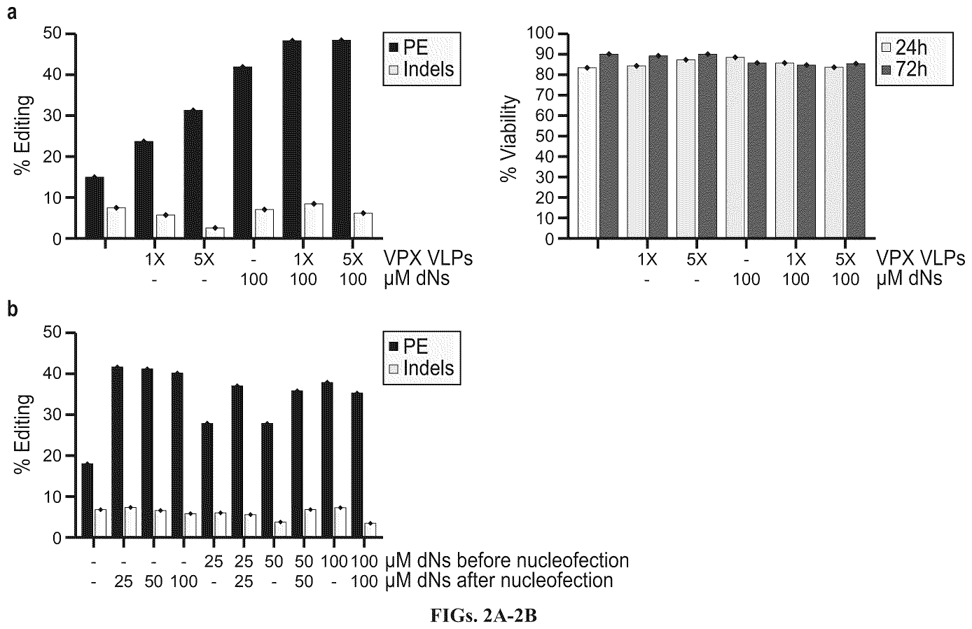

HSPCs were thawed and cultured with or without 100 μM each deoxynucleosides (dNs). After 24 hours of culture, 5×10⁵ HSPCs were electroporated with 2000 ng Prime Editor 2 (PE2) mRNA+200 pmol synthetic ATP1A1-Q118R epegRNA+100 pmol synthetic nick sgRNA using pulse code DS-130. HSPCs were then cultured for 72 hours in the presence or absence of 100 μM dNs and VPX VLPs. 1× lentiBOOST™ was used in all conditions. DMEM supplemented with 10% FBS and 1% Pen/Strep was used as a vehicle control for VPX VLPs. Genomic DNA was harvested 3 days post-nucleofection. Genomic DNA was harvested 3 days post-nucleofection and sequenced to detect the presence or absence of edits. As expected, a Q118R mutation was installed at ATP1A1 using the prime editing system 3 (PE3). Interestingly, when prime editing was carried out in the presence of VPX and deoxynucleosides, and increase in prime editing efficacy was observed (FIG. 2).

PE and indels were quantified using BEAT and tracking of indels by decomposition (TIDE) analysis from Sanger sequences, respectively. Cells were counted using a Neubauer chamber and viability was measured via Trypan blue staining. Significantly, prime editing carried out in the presence of 5×VPX VLPs and 100 uM deoxynucleosides was significantly more effective than when prime editing was carried out in the absence of the VPX VLPs and added deoxynucleosides. The results are displayed in Table 1 (below).

TABLE 1

| | | | 24 h post- | | 72 h post- | |
|---|---|---|---|---|---|---|
| Nucleo- | dNs | VPX | nucleofection | | nucleofection | |
| fection | (μM) | VLPs | Cells/ml | % Viability | Cells/ml | % Viability |
| 1 | — | — | 530 000 | 83.5 | 1 675 000 | 90.1 |
| 2 | — | 1X | 705 000 | 84.4 | 1 825 000 | 89.5 |
| 3 | — | 5X | 605 000 | 87.7 | 1 720 000 | 90.1 |
| 4 | 100 | — | 465 000 | 88.6 | 1 065 000 | 85.9 |
| 5 | 100 | 1X | 485 000 | 85.8 | 955 000 | 84.9 |
| 6 | 100 | 5X | 490 000 | 83.8 | 1 180 000 | 85.5 |

### Example 3: The Addition of Deoxynucleosides (dNs) Enhanced Prime Editing in Resting and Activated CD3+ T Cells

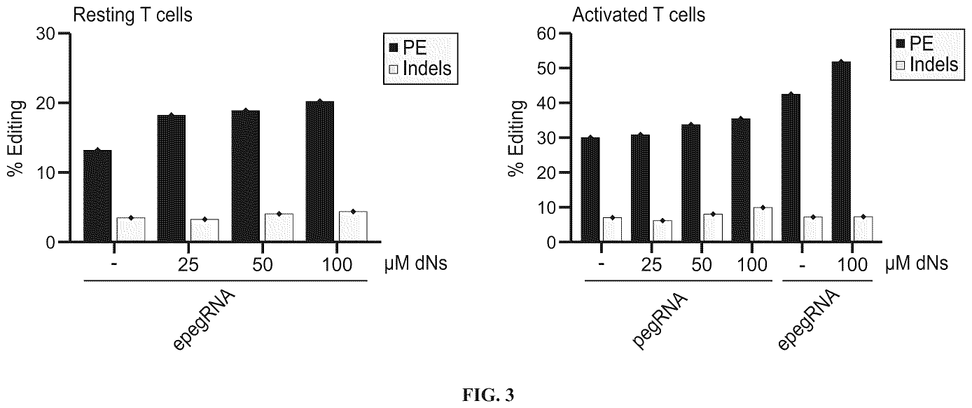

After 24 hours of culture, resting CD3⁺ T cells were either electroporated or activated for 48 hours with CD3/CD28 dynabeads. 5×10⁵ resting CD3⁺ T cells were electroporated with 2000 ng PE2 mRNA+200 pmol synthetic ATP1A1-Q118R epegRNA+100 pmol synthetic nick sgRNA using pulse code EH-115. CD3⁺ resting T cells were then cultured for 72 hours with 300 U/ml IL-2 in the presence or absence of dNs. Genomic DNA was harvested 3 days post-nucleofection. For activated T cells, 5×10⁵ CD3⁺ T cells were electroporated after 48 hours of activation with 2000 ng PE2 mRNA+200 pmol synthetic ATP1A1-Q118R epegRNA+100 pmol synthetic nick sgRNA using pulse code EH-115. CD3⁺ T cells were then cultured for 72 hours with 300 U/ml IL-2 in the presence or absence of dNs. Genomic DNA was harvested 3 days post-nucleofection. The results are illustrated in FIG. 3.

### Example 4: Nucleotide Metabolism Constrains Prime Editing in Hematopoietic Stem and Progenitor Cells

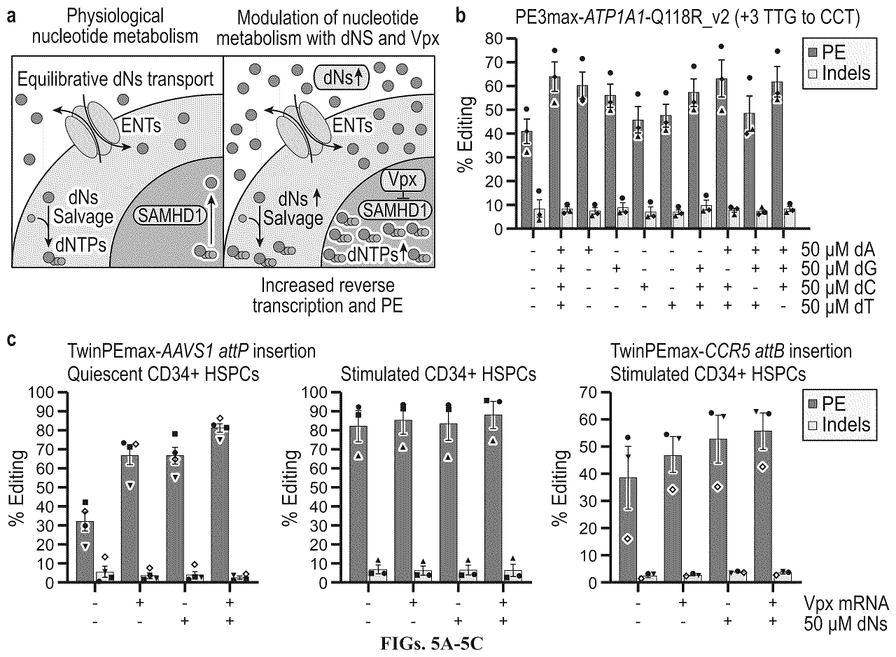

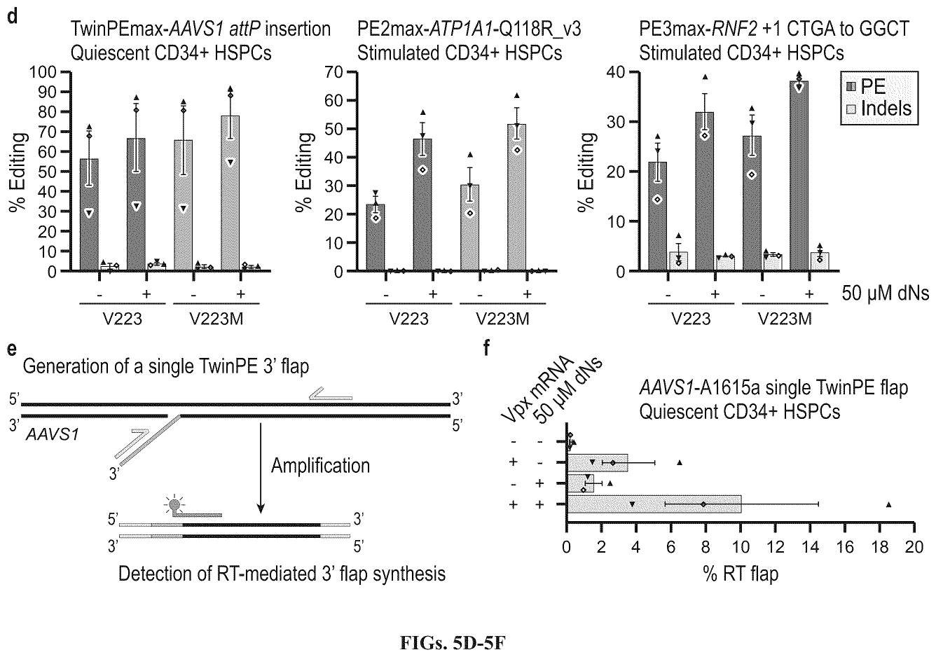

Prime editing (PE) relies on a Cas9 nickase fused to a reverse transcriptase along with an extended prime editing guide RNA (pegRNA) to introduce to living cells templated genomic modifications, including point mutations, short insertions and deletions (indels), and longer sequence replacements. While recent advances have improved the technology, efficient prime editing remains challenging in primary hematopoietic cells. One key difference between cancer cell lines and primary hematopoietic cells is the concentration of nucleotides available for reverse transcription. Nondividing cells typically have orders of magnitude lower nucleotide levels as compared to dividing cells. SAMHD1 is a triphosphohydrolase enzyme that depletes deoxyribonucleoside triphosphates (dNTPs) and acts as an antiviral factor to restrict HIV-1 infection. Since SAMHD1 expression markedly increases in hematopoietic stem and progenitor cells (HSPCs) after cytokine culture, it was hypothesized that modulating the nucleotide metabolism could enhance reverse transcription and thus prime editing (FIG. 5A).

The accessory lentiviral protein VPX, encoded by HIV-2 and SIV viruses, associates with the CRL4^{DCAF1} E3 ubiquitin ligase to target SAMHD1 for proteasomal degradation. It was first tested whether delivering VPX via virus-like particles (VLPs) transduction could enhance prime editing. HSPCs were cultured in the presence of cytokines for 24 hours, and electroporated cells with a first-generation prime editor (PE2) mRNA and a previously optimized synthetic epegRNA/nicking sgRNA pair targeting ATP1A1. Following electroporation, HSPCs were cultured for 72 hours in the presence or absence of VPX VLPs. The frequency of alleles harboring the ATP1A1-Q118R mutation increased from an average of 17% to 31% with HSPCs from two different healthy donors, as determined by Sanger sequencing (FIGS. 1-2).

Since HSPCs lack the capacity to carry out de novo nucleotide synthesis and rely on membrane transporters such as ENT1 to ensure extracellular nucleoside uptake and dNTP synthesis via the nucleoside salvage pathway, it was hypothesized that exogenous deoxynucleoside (dN) supplementation could further boost prime editing (FIG. 5A). HSPCs were treated with 100 μM of each dN before and after electroporation and observed an increase in prime editing efficiency from 15% to 42% without VPX VLPs, and to 48% with VPX VLPs (FIGS. 1-2). Cells were then treated with different concentrations of dNs before, after, and both before and after electroporation. Although dNs enhanced PE with all conditions, higher improvements (from 18% to 42%) were observed when supplementing dNs after electroporation (FIGS. 1-2). For subsequent experiments, cells were treated with or without 50 μM of each dN after electroporation to maximize prime editing efficiency.

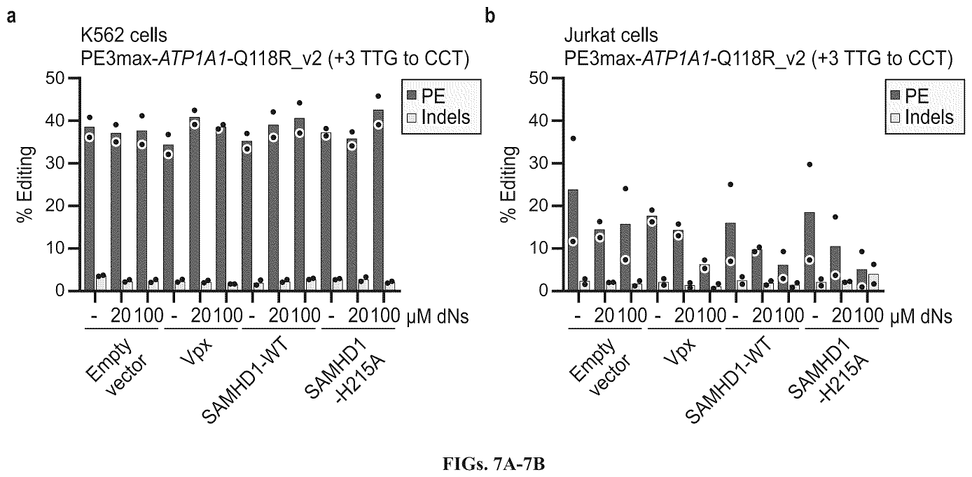

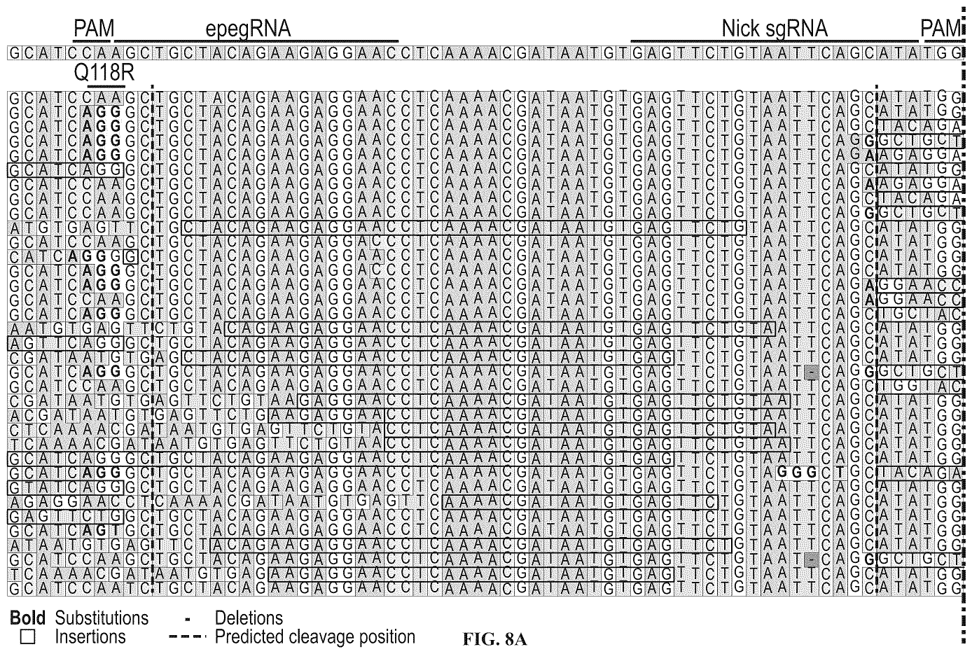

It was then tested whether all four dNs were needed for maximal PE enhancement. To further boost efficiency, PEmax² mRNA was generated, which markedly increased the basal level of editing to an average of 41% ATP1A1-Q118R allele frequency across HSPCs from three different donors (FIG. 5B). An increase was observed with all individual dNs as well as all dNs minus one conditions, but the highest level of editing was observed with all four dNs to an average of 64% edited alleles (FIG. 5B). Since all four dNTPs are needed to synthesize the ATP1A1-Q118R epegRNA flap, these results are consistent with the hypothesis that the dNTP concentration is limiting for efficient reverse transcription and PE in HSPCs. The impact of dNs supplementation and overexpression of VPX, SAMHD1, or the inactive SAMHD1-H215A variant on PE in K562 and Jurkat cells was also tested. Modulating the nucleotide metabolism had little to no impact on editing efficiency, suggesting that the concentration of dNTPs is not limiting in cancer cell lines (FIG. 7).

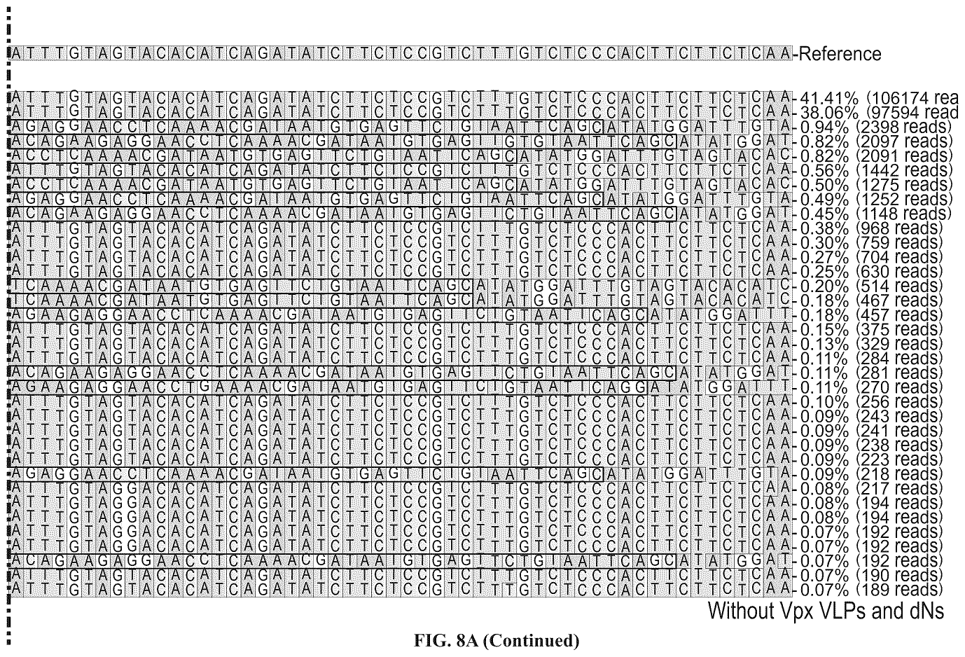

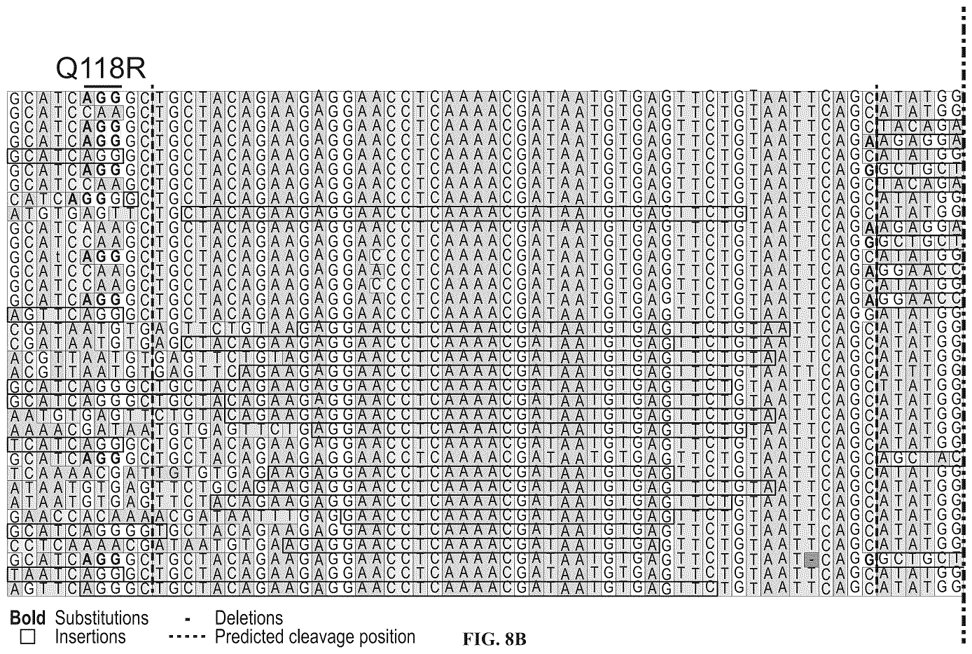

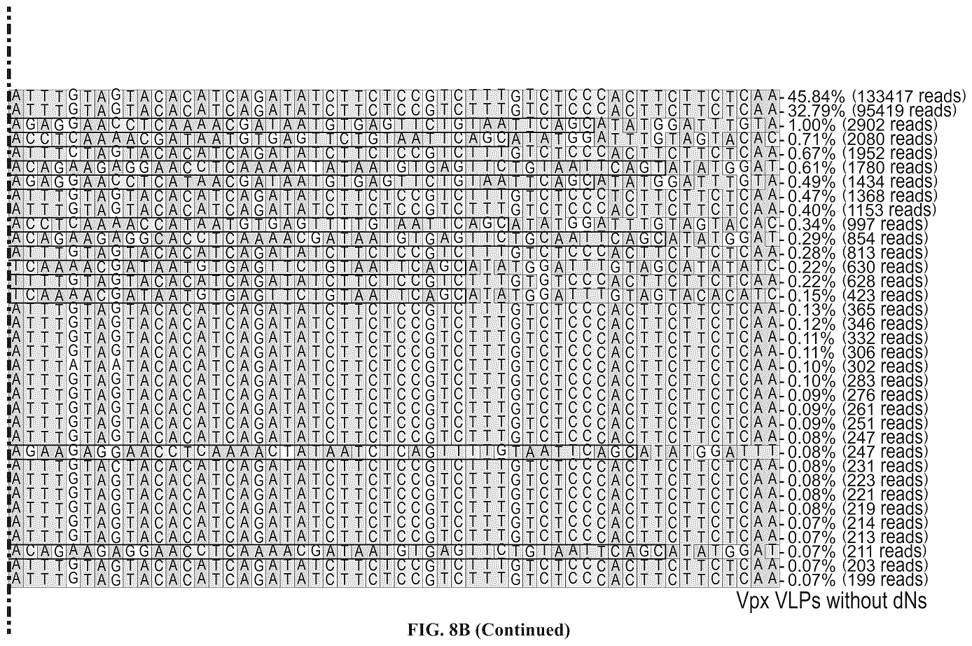

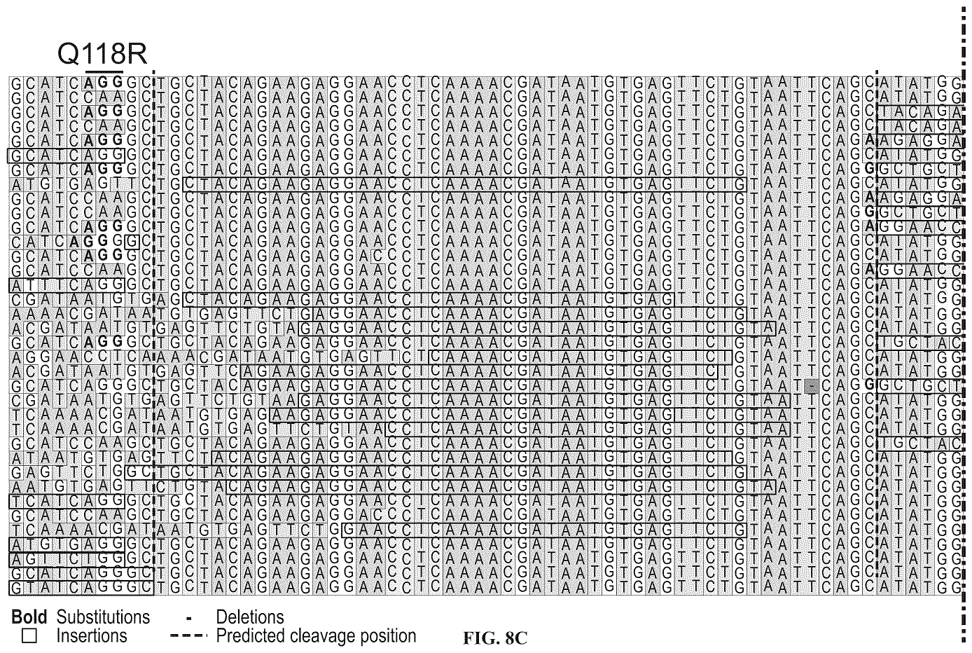

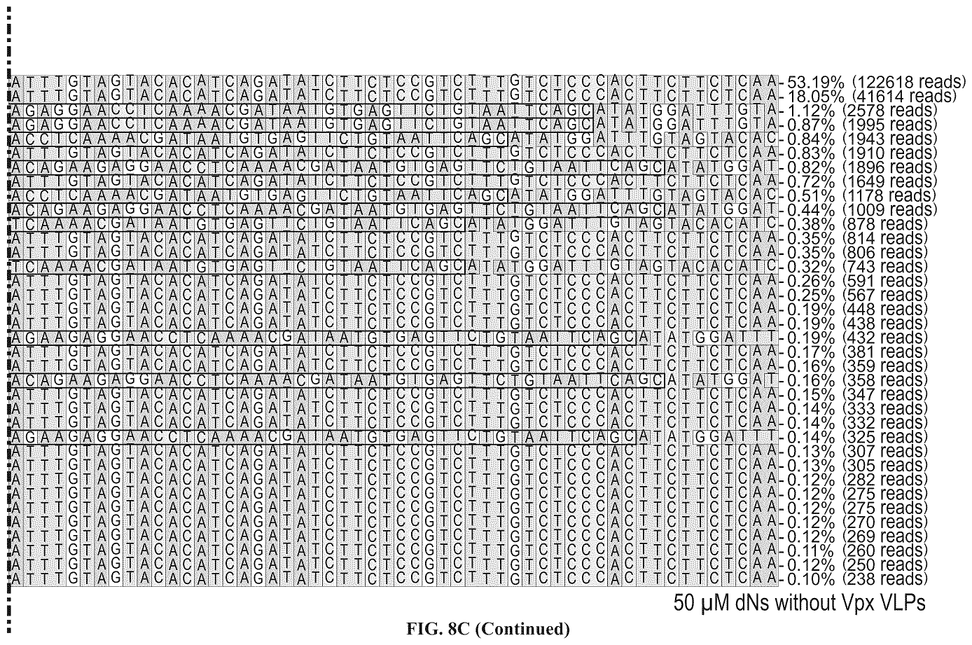

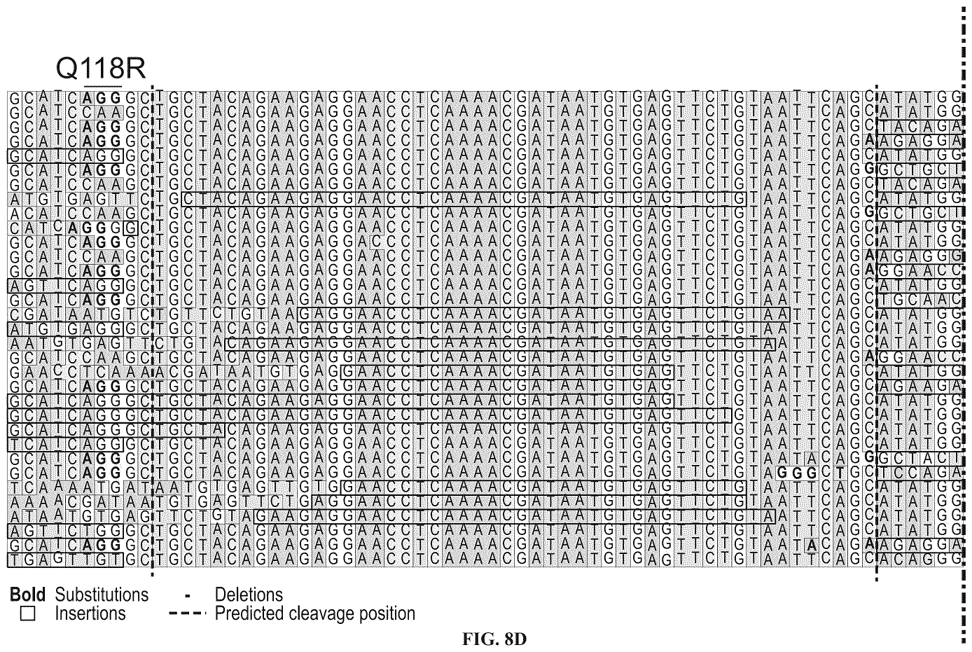

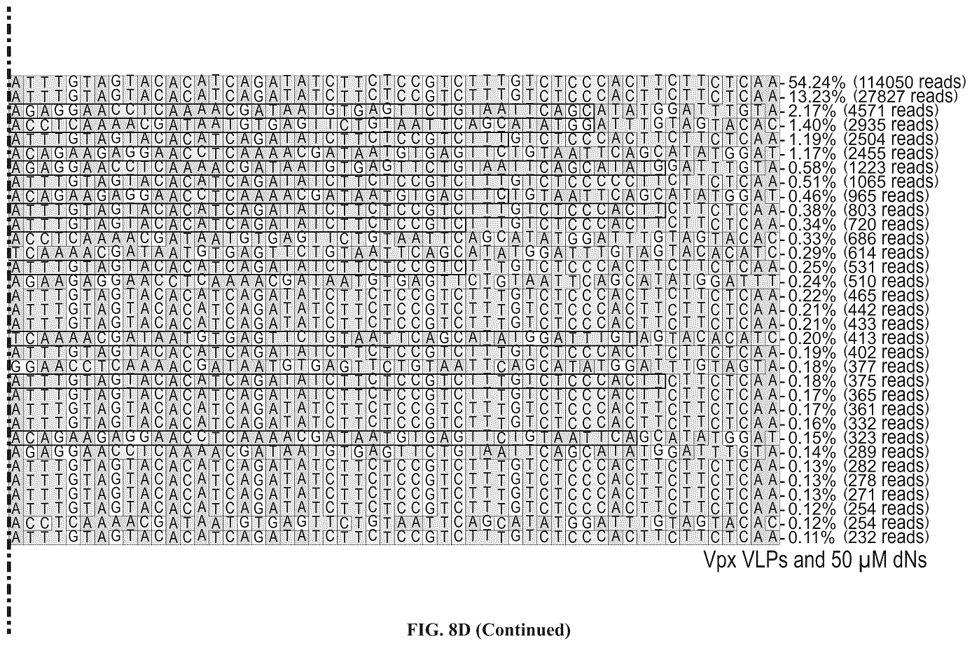

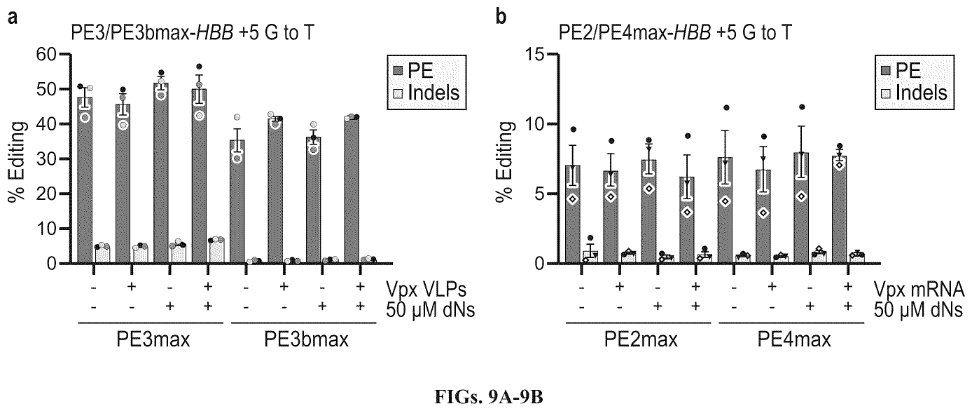

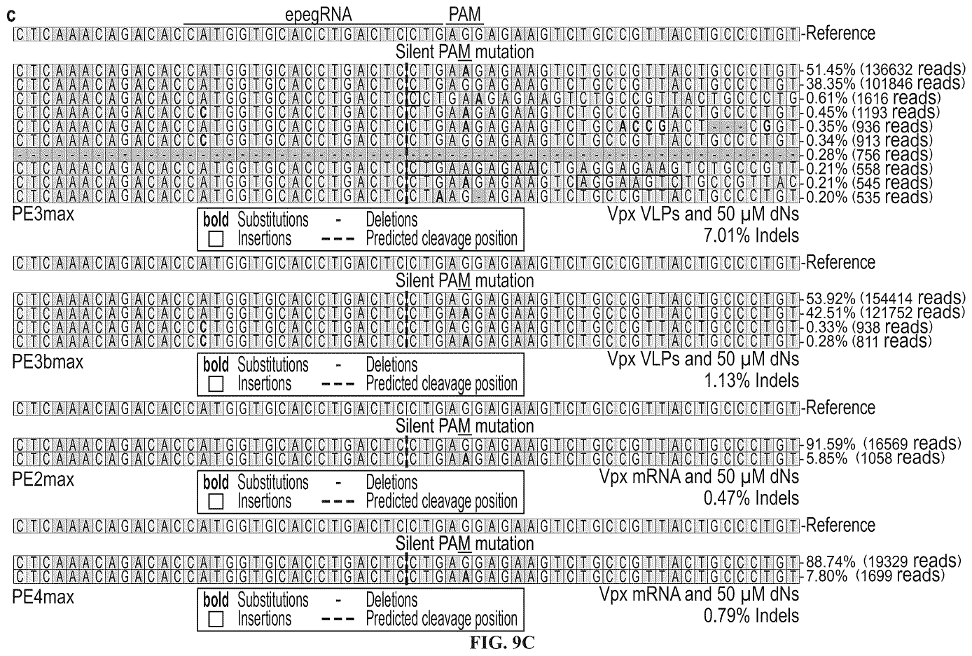

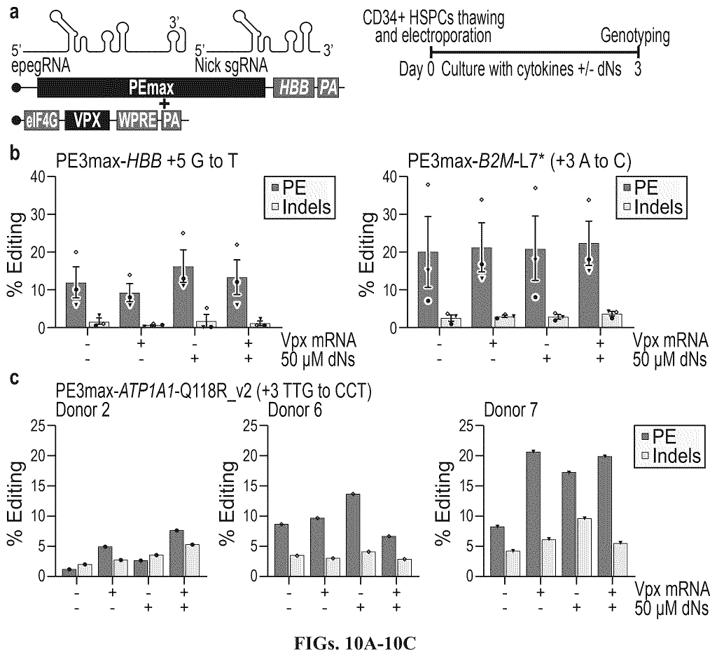

The impact of dNs supplementation and VPX VLPs was assessed using an additional epegRNA designed to correct the HBB-E6V mutation causative of sickle cell disease using PE3max and PE3bmax strategies. While Vpx VLP and dN treatments improved PE at ATP1A1 (FIG. 8), modest improvements were observed at the HBB locus with the PE3max strategy (FIG. 9). Nonetheless, the average level of editing increased from 35% to 42% with an average of just 1.25% indels with the PE3bmax strategy, as determined by amplicon sequencing (FIG. 9). This level of product purity contrasted with the high level of tandem duplications observed at ATP1A1 with the PE3max strategy in the presence or absence of dNs and Vpx VLPs (FIG. 8), corroborating previous findings in K562 cells with pegRNA and nicking sgRNA PAMs facing outward towards each other (PAM-out configuration) (Levesque et al. Nat. Commun. 13, 5909 (2022)). Of relevance, hMLH1dn mRNA co-delivery did not improve PE in HSPCs, as previously observed (Everette et al., Nat. Biomed. Eng. 7, 616-628 (2023)). (FIG. 9). Editing quiescent HSCs without cytokine culture reduces DSB genotoxicity and may ultimately enable editing HSCs in vivo. It was hypothesized that the nucleotide pool available for reverse transcription could especially limit PE in quiescent HSPCs that had not been stimulated by cytokines in vitro. Vpx mRNA was also generated to co-deliver at an equimolar ratio with PEmax mRNA during electroporation to confirm that Vpx alone could ameliorate PE (FIG. 10A). Electroporation was performed directly after thawing CD34+ HSPCs, and cells were cultured for 72 hours in the presence or absence of 50 μM dNs (FIG. 10A). While Vpx mRNA co-delivery and dN supplementation improved editing at ATP1A1, modulation of nucleotide metabolism had minimal impact on PE in quiescent HSPCs with the HBB epegRNA and an additional epegRNA designed to knockout B2M (Fiumara et al., Nat. Biotechnol. doi: 10.1038/s41587-023-01915-4 (2023)). (FIGS. 10B-10C). Of relevance, it was noted that the highest level of improvement was observed with the ATP1A1-Q118R_v2 epegRNA, which was optimized to evade DNA mismatch repair (MMR) (Levesque et al. Nat. Commun. 13, 5909 (2022)) while the HBB and B2M epegRNAs introduce a single nucleotide substitution and are thus sensitive to MMR. Since MMR antagonizes prime editing efficiency and fidelity (Chen et al., Cell 184, 5635-5652 (2021); Ferreira da Silva et al. Nat. Commun. 13, 760 (2022)), it was hypothesized that modulating the nucleotide metabolism could positively interact with MMR evasion to facilitate prime editing in HSPCs.

Twin prime editing (TwinPE) generates 3′ DNA flaps dissimilar to the target site and is not expected to produce heteroduplexes that engage MMR (Anzalone et al., Nat. Biotechnol. 40, 731-740 (2022)). The impact of modulating nucleotide metabolism on TwinPE was tested using pairs of (e) pegRNAs designed to install Bxb1 attP or attB recombinase sites at the AAVS1 and CCR5 loci, respectively (Anzalone et al., Nat. Biotechnol. 40, 731-740 (2022)). Vpx mRNA co-delivery and dN supplementation markedly improved TwinPE efficiency from an average of 32% to 81% at the AAVS1 locus in quiescent HSPCs from four donors, and from an average of 39% to 56% at the CCR5 locus in stimulated HSPCs from three donors (FIG. 5C). Consistent with the hypothesis of dNTPs being especially limiting in quiescent HSPCs, a higher basal level of twin prime editing and a reduced improvement with Vpx mRNA and dN supplementation were observed at AAVS1 in HSPCs cultured with cytokines before electroporation (FIG. 5C). Since TwinPE bypasses multiple DNA repair steps that can reject traditional prime edits, including 3′ flap annealing to the genome and heteroduplex resolution (Anzalone et al., Nat. Biotechnol. 40, 731-740 (2022); Chen & Liu, Nat. Rev. Genet. 24, 161-177 (2022); Nambiar et al., Mol. Cell 82, 348-388 (2022)), these results suggested that MMR evasion interacts positively with dN supplementation and Vpx to boost prime editing efficiency.

To substantiate these findings, it was tested whether the limited level of dNTPs constrains reverse transcription in HSPCs. The V223M mutation known to increase dNTP affinity (Skasko et al. J. Biol. Chem. 280, 12190-12200 (2005); Sharma et al., Antivir. Chem. Chemother. 16, 169-182 (2005); Palikša et al., Protein Eng. Des. Sel. 31, 79-89 (2018); Ponnienselvan et al., BioRxiv. Preprint at https://www.biorxiv.org/content/10.110 (2023)) was introduced to the M-MLV reverse transcriptase, generating the PEmax-V223M variant. This variant improved PE at three different loci in quiescent and stimulated HSPCs from three donors (FIG. 5D). While the PEmax-V223M variant enhanced PE to a lower extent than dNs supplementation in stimulated HSPCs, both strategies had an additive impact on PE, further expanding the toolkit available to overcome this metabolic constraint. A digital droplet PCR (ddPCR) assay was developed to measure PE-mediated RT based on the synthesis of a non-homologous 3′ ssDNA flap that does not integrate to its target locus (FIG. 5E). Quiescent HSPCs were electroporated and genomic DNA was harvested 6 hours post-nucleofection. A 37-fold increase on average was observed in the PE-mediated RT products at the AAVS1 locus in HSPCs from three different donors (FIG. 5F). Amplicon sequencing was performed and flap integration events were detected in only 1 out of 12 samples, confirming the amplification of non-integrated 3′ ssDNA flap intermediates via ddPCR. Altogether, these results showed that nucleotide metabolism constrains reverse transcription and restricts prime editing in HSPCs.

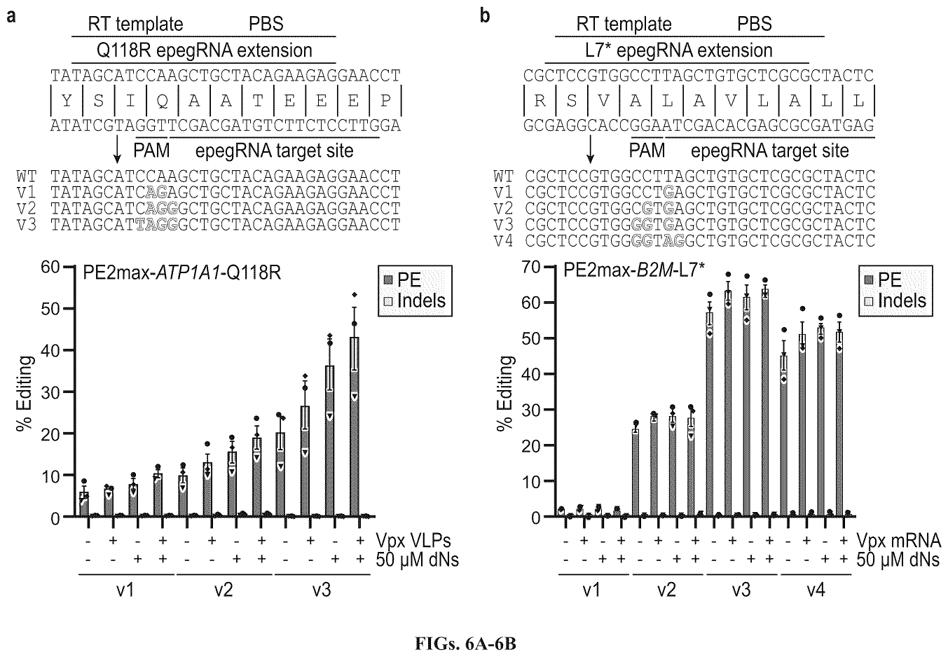

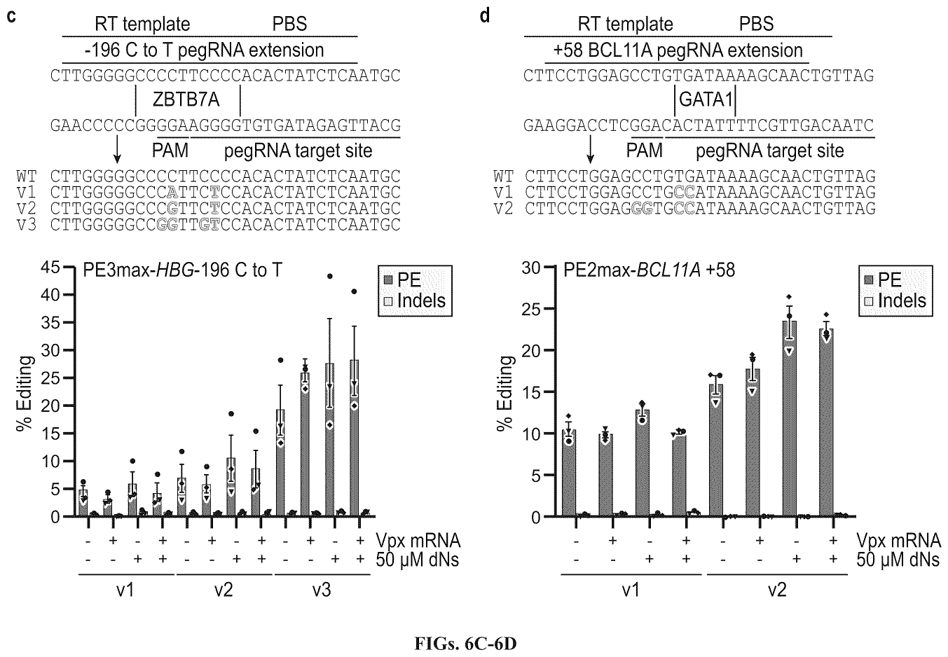

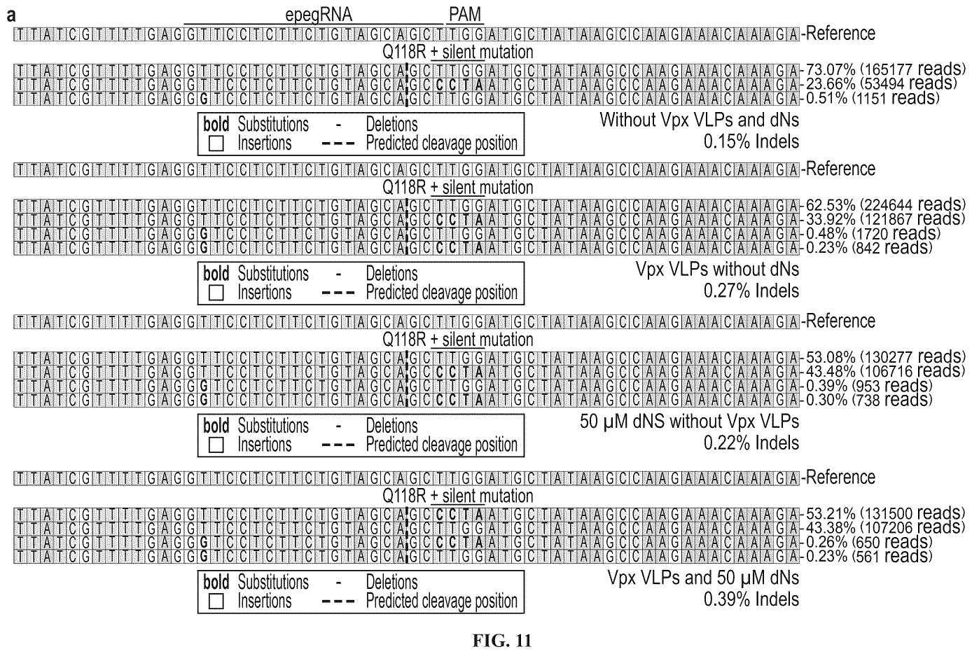

Considering the lower product purity observed when using an additional nicking sgRNA, it was tested whether modulating the nucleotide metabolism could positively interact with MMR evasion to achieve high levels of editing with the PE2max approach. A simple yet very efficient approach to evade MMR is to install additional silent mutations near the intended edit (Chen et al., Cell 184, 5635-5652 (2021); Li et al., Nat. Commun. 13, 1669 (2022)), as previously demonstrated with ATP1A1-Q118R v1-v3 epegRNAs (Levesque et al. Nat. Commun. 13, 5909 (2022)) (FIG. 6A). In HSPCs, a marked increase in PE2max efficiency was observed with three (v2) and four (v3) substitutions compared to two (v1) using ATP1A1-Q118R_v1-v3 epegRNAs (FIG. 6A), corroborating previous findings in K562 cells (Levesque et al. Nat. Commun. 13, 5909 (2022)). Treatment with Vpx VLPs and dNs further enhanced precise prime editing from an average of 20.1% to 42.9% allele frequency with the v3 epegRNA, allowing up to 53.2% PE alleles with 0.4% indels and completely abrogating tandem duplications, as determined by amplicon sequencing, i.e. >100 PE-to-indel purity ratio (FIGS. 6A, 11). Additional (e) pegRNAs were designed introducing C·C mismatches which are resistant to MMR (Su et al., J. Biol. Chem. 263, 6829-6835 (1988); Thomas et al., J. Biol. Chem. 266, 3744-3751 (1991); Lahue et al., Science 245, 160-164 (1998)) (FIGS. 6B-D). Strikingly, PE efficiency rose from an average of 2% with the original B2M epegRNA to an average of 25% and 57% with one and two C·C mismatches, respectively (FIG. 6B). Together, MMR evasion and nucleotide metabolism modulation allowed up to 68% prime editing at B2M (FIG. 6B). Co-delivering Vpx mRNA and supplementing dNs also increased PE selectively with MMR-evading pegRNAs targeting the HBG1/2 promoters and the +58 BCL11A enhancer (FIGS. 6C-6D). These data demonstrated that MMR opposes prime editing in HSPCs and that modulating nucleotide metabolism positively interacts with MMR-evasion to install prime edits with high purity and efficiency in the absence of additional nicking sgRNAs using the PE2max system.

The enhancements observed in this study suggest that the low concentration of dNTPs available for reverse transcription restricts prime editing in HSPCs. Modulating nucleotide metabolism is a straightforward and effective approach to ameliorate prime editing in therapeutically-relevant cell types, including primary cells where the cellular concentration of dNTPs is several-fold lower in primary than cancer cells (Mathews, C. K. Nat. Rev. Cancer 15, 528-539 (2015); Traut, T. W. Mol. Cell. Biochem. 140, 1-22 (1994)) and quiescent cells which have orders of magnitude lower nucleotide levels as compared to dividing cells. The marked positive interactions between the modulation of nucleotide metabolism and TwinPE could be interpreted that boosting the level of dNTPs may facilitate both reverse transcription and the DNA polymerase fill-in synthesis step required to install TwinPE modifications. Paradoxically, dNTP availability could also boost MMR, which depends on fill-in synthesis by DNA polymerase 8 after the excision of the mismatched DNA strand, and antagonize PE by restoring the original sequence more efficiently. This could partially explain the greater increase in PE efficiency observed with modulating nucleotide metabolism along with MMR-evading mutations or twin prime editing.

Altogether, modulation of nucleotide metabolism enhances prime editing in HSPCs which should facilitate clinical translation of therapeutic prime editing. The present disclosure contemplates that delivery of Vpx and/or RT variants as mRNA or VLP are expected to be straightforward approaches to implement with current modalities and even systemic deoxynuceloside therapy is contemplated. Combining modulation of nucleotide metabolism, PEmax editors, and MMR evasion designs allows unprecedented prime editing efficiency and purity in HSPCs. Considering recent progress towards delivery of genome editors to HSCs in vivo, these findings may help designing novel prime editing therapies to engineer quiescent HSCs in situ.

The results described herein above were carried out using the following methods and materials.

### Human Hematopoietic Stem and Progenitor Cells (HSPCs) and Primary CD3+ T Cells Culture

Cryopreserved human CD34+ HSPCs from mobilized peripheral blood of deidentified healthy donors were obtained from the Fred Hutchinson Cancer Research Center (Seattle, Washington). CD34+ HSPCs were cultured with X-Vivo-15 media supplemented with 100 ng/ml human Stem Cell Growth Factor (SCF), 100 ng/ml human thrombopoietin (TPO), and 100 ng/ml recombinant human FMS-like Tyrosine Kinase 3 Ligand (Flt3-L). K562 (CCL-243) and Jurkat (TIB-152) cells were obtained from the ATCC and cultured at 37° C. under 5% CO₂ in RPMI media supplemented with 10% FBS, and 1% Penicillin/Streptomycin. HEK293T (CRL-1573) cells were obtained from ATCC and cultured at 37° C. under 5% CO₂ in DMEM media supplemented with 10% FBS, and 1% Penicillin/Streptomycin. Deoxynucleosides (dA, Sigma-Aldrich D8668) (dG, Sigma-Aldrich D0901) (dC, Sigma-Aldrich D0776) (dT, Sigma-Aldrich T1895) were resuspended in water at 12.5 mM each, filter-sterilized, and stored at −20° C.

### Vpx Virus-Like Particles (VLPs)

Vpx VLPs were provided by the Genetic Perturbation Platform (GPP) of the Broad Institute of MIT and Harvard or produced in-house. Briefly, HEK293T cells were transfected with VSV-G envelope and SIV Vpx vectors, and virus-like particles were harvested from the supernatant 35-40 hours post-lipofection. The 1× concentration (1/20 of the culture volume) was established as the minimal volume to enhance lentiviral transduction of primary human monocytes. Titration of activity was performed using prime editing and cell viability as readouts, and a concentration of 5× (1/4 of the culture volume) was used for all experiments using GPP VLPs. HEK293T culture media was used as a vehicle control in all experiments using GPP VLPs. Alternatively, in-house VLPs were produced using the same protocol followed by ultracentrifugation at 24,000 RPM for 2 hours (SW 28 Beckman swinging bucket rotor) to concentrate the VLPs and avoid diluting the HSPCs culture media. Titration of activity was performed using prime editing and cell viability as readouts, and a concentration of 25×Vpx VLPs (normalized from the initial volume of producer cell line supernatant) was used for all experiments using in-house VLPs.

In Vitro Transcription, and epegRNA and Nicking sgRNA Synthesis

The PE2 and PEmax transcription template plasmids were linearized, and mRNA was transcribed using the HiScribe T7 high yield RNA kit (NEB) using NI-methylpseudouridine (Trilink) instead of uridine, and co-transcriptional capping with CleanCap AG (Trilink). The PEmax in vitro transcription plasmid template derives from a previously reported vector (Casirati, et al. Nature 621, 404-414 (2023)) and encodes for a T7 promoter, a minimal 5′-UTR, a PEmax cassette harboring a silent mutation disrupting a restriction site for the linearizing BbsI enzyme, a 2×HBB 3′ UTR, and a 110-120 bp poly (A) sequence. For SIV Vpx mRNA IVT template, a cassette harboring a 5′ UTR region with an eIF4G aptamer, a kozak sequence, the Vpx cassette, and a 3′ UTR harboring the WPRE element was cloned in the pT7-PEmax for IVT plasmid (Addgene 178113). The template was generated as previously described in Chen, P. J. et al, Cell 184, 5635-5652 (2021). Briefly, the template was PCR amplified with a forward primer that correct a T7 promoter inactivating mutation and a reverse primer that appends a 119-nt poly (A) tail (SEQ ID NO: 5) to the 3′ UTR. Following IVT, mRNAs were purified using the Monarch RNA Cleanup kit (500 μg) (NEB) and eluted in 1× nuclease-free IDTE buffer (10 mM Tris, 0.1 mM EDTA, pH 7.5). The mRNA concentration was quantified using Qubit RNA high sensitivity (HS) kit (ThermoFisher). Synthetic pegRNAs and epegRNAs were provided by Integrated DNA Technologies (IDT) and resuspended at 200 pmol/μl in nuclease-free IDTE buffer (10 mM Tris, 0.1 mM EDTA, pH 7.5). The pegRNAs and epegRNAs contained 2′-O-methyl modifications, and phosphorothioate linkages. Synthetic nicking sgRNAs were provided by IDT at a 10 nmol scale and resuspended at 200 pmol/μl in nuclease-free IDTE buffer (10 mM Tris, 0.1 mM EDTA, pH 7.5). All pegRNA, epegRNA, and nicking sgRNA sequences and chemical modifications are provided in Table 2, below.

TABLE 2
epegRNAs, pegRNAs, and Nicking sgRNAs Used

| epegRNA name | Sequence |
|---|---|
| ATPIAI_Q118R_v1 | mG*mU*mU*CCUCUUCUGUAGCAGCUGUUUUAGAGCUAGAAAUAGCAAGUUAAAAUAAGGCUAGUC |
| | CGUUAUCAACUUGAAAAAGUGGCACCGAGUCGGUGCUAGCAUCAGAGCUGCUACAGAAGAGCGCGG |
| | UUCUAUCUAGUUACGCGUUAAACCAACUAGAA*mU*mU*mU (SEQ ID NO: 6) |
| ATPIAI_Q118R_v2 | mG*mU*mU*CCUCUUCUGUAGCAGCUGUUUUAGAGCUAGAAAUAGCAAGUUAAAAUAAGGCUAGUC |
| | CGUUAUCAACUUGAAAAAGUGGCACCGAGUCGGUGCUAGCAUCAGGGCUGCUACAGAAGAGCGCGG |
| | UUCUAUCUAGUUACGCGUUAAACCAACUAGAA*mU*mU*mU (SEQ ID NO: 7) |
| ATPIAI_Q118R_v3 | mG*mU*mU*CCUCUUCUGUAGCAGCUGUUUUAGAGCUAGAAAUAGCAAGUUAAAAUAAGGCUAGUC |
| | CGUUAUCAACUUGAAAAAGUGGCACCGAGUCGGUGCUAGCAUUAGGGCUGCUACAGAAGAGCGCGG |
| | UUCUAUCUAGUUACGCGUUAAACCAACUAGAA*mU*mU*mU (SEQ ID NO: 8) |
| HBB_E6V_Correction | mC*mA*mU*GGUGCACCUGACUCCUGGUUUUAGAGCUAGAAAUAGCAAGUUAAAAUAAGGCUAGUC |
| | CGUUAUCAACUUGAAAAAGUGGCACCGAGUCGGUGCAGACUUCUCUUCAGGAGUCAGGUGCACAGA |
| | AUAAACGCGGUUCUAUCUAGUUACGCGUUAAACCAACUAGAA*mU*mU*mU (SEQ ID NO: 9) |
| AAVSI_A1615a_TwinPE | mG*mC*mA*GCUCAGGUUCUGGGAGAGUUUUAGAGCUAGAAAUAGCAAGUUAAAAUAAGGCUAGUC |
| | CGUUAUCAACUUGAAAAAGUGGCACCGAGUCGGUGCUACCGUACACCACUGAGACCGCGGUGGUUG |
| | ACCAGACAAACCUCCCAGAACCCGCGGUUCUAUCUAGUUACGCGUUAAACCAACUAGAA*mU*mU*m |
| | U (SEQ ID NO: 10) |
| AAVSI_B1705b_TwinPE | mG*mA*mU*GGAGCCAGAGAGGAUCCGUUUUAGAGCUAGAAAUAGCAAGUUAAAAUAAGGCUAGUC |
| | CGUUAUCAACUUGAAAAAGUGGCACCGAGUCGGUGCGUCUGGUCAACCACCGCGGUCUCAGUGGUG |
| | UACGGUACAAACCUUCCUCUCUGGCCGCGGUUCUAUCUAGUUACGCGUUAAACCAACUAGAA*mU*m |
| | U*mU (SEQ ID NO: 11) |
| B2M_L7Stop_v1 | mG*mA*mG*UAGCGCGAGCACAGCUAGUUUUAGAGCUAGAAAUAGCAAGUUAAAAUAAGGCUAGUC |
| | CGUUAUCAACUUGAAAAAGUGGCACCGAGUCGGUGCUCCGUGGCCUGAGCUGUGCUCGCGCCGCGG |
| | UUCUAUCUAGUUACGCGUUAAACCAACUAGAA*mU*mU*mU (SEQ ID NO: 12) |
| B2M_L7Stop_v2 | mG*mA*mG*UAGCGCGAGCACAGCUAGUUUUAGAGCUAGAAAUAGCAAGUUAAAAUAAGGCUAGUC |
| | CGUUAUCAACUUGAAAAAGUGGCACCGAGUCGGUGCUCCGUGGCGUGAGCUGUGCUCGCGCCGCGG |
| | UUCUAUCUAGUUACGCGUUAAACCAACUAGAA*mU*mU*mU (SEQ ID NO: 13) |
| B2M_L7Stop_v3 | mG*mA*mG*UAGCGCGAGCACAGCUAGUUUUAGAGCUAGAAAUAGCAAGUUAAAAUAAGGCUAGUC |
| | CGUUAUCAACUUGAAAAAGUGGCACCGAGUCGGUGCUCCGUGGGGUGAGCUGUGCUCGCGCCGCGG |
| | UUCUAUCUAGUUACGCGUUAAACCAACUAGAA*mU*mU*mU (SEQ ID NO: 14) |
| B2M_L7Stop_v4 | mG*mA*mG*UAGCGCGAGCACAGCUAGUUUUAGAGCUAGAAAUAGCAAGUUAAAAUAAGGCUAGUC |
| | CGUUAUCAACUUGAAAAAGUGGCACCGAGUCGGUGCUCCGUGGGGUAGGCUGUGCUCGCGCCGCGG |
| | UUCUAUCUAGUUACGCGUUAAACCAACUAGAA*mU*mU*mU (SEQ ID NO: 15) |
| pegRNA name | Sequence |
| CCR5_A531c_v2_TwinPE | mG*mC*mU*GUGUUUGCGUCUCUCCCGUUUUAGAGCUAGAAAUAGCAAGUUAAAAUAAGGCUAGUC |
| | CGUUAUCAACUUGAAAAAGUGGCACCGAGUCGGUGCAUCCUGACGACGGAGACCGCCGUCGUCGAC |
| | AAGCCAGAGACGCAAACAUU*mU*mU*mUU (SEQ ID NO: 16) |
| CCR5_B584b_v2_TwinPE | mG*mU*mA*UGGAAAAUGAGAGCUGCGUUUUAGAGCUAGAAAUAGCAAGUUAAAAUAAGGCUAGUC |
| | CGUUAUCAACUUGAAAAAGUGGCACCGAGUCGGUGCUUGUCGACGACGGCGGUCUCCGUCGUCAGG |
| | AUCAUGCUCUCAUUUUCUU*mU*mU*mUU (SEQ ID NO: 17) |
| RNF2_+1_CTGA_To_GGCT | mG*mU*mC*AUCUUAGUCAUUACCUGGUUUUAGAGCUAGAAAUAGCAAGUUAAAAUAAGGCUAGUC |
| | CGUUAUCAACUUGAAAAAGUGGCACCGAGUCGGUGCAACGAACACCAGCCGUAAUGACUAAGAUGU |
| | U*mU*mU*mUU (SEQ ID NO: 18) |
| HBG- | mG*mC*mA*UUGAGAUAGUGUGGGGAGUUUUAGAGCUAGAAAUAGCAAGUUAAAAUAAGGCUAGUC |
| 196_C_To_T_v1 | CGUUAUCAACUUGAAAAAGUGGCACCGAGUCGGUGCUUGGGGGCCCAUUCUCCACACUAUCUCAUU* |
| | mU*mU*mUU (SEQ ID NO: 19) |
| HBG- | mG*mC*mA*UUGAGAUAGUGUGGGGAGUUUUAGAGCUAGAAAUAGCAAGUUAAAAUAAGGCUAGUC |
| 196_C_To_T_v2 | CGUUAUCAACUUGAAAAAGUGGCACCGAGUCGGUGCUUGGGGGCCCGUUCUCCACACUAUCUCAUU* |
| | mU*mU*mUU (SEQ ID NO: 20) |
| HBG- | mG*mC*mA*UUGAGAUAGUGUGGGGAGUUUUAGAGCUAGAAAUAGCAAGUUAAAAUAAGGCUAGUC |
| 196 C To T v3 | CGUUAUCAACUUGAAAAAGUGGCACCGAGUCGGUGCUUGGGGGCCGGUUGUCCACACUAUCUCAUU* |
| | mU*mU*mUU (SEQ ID NO: 21) |
| BCL11A_+58_v1 | mC*mU*mA*ACAGUUGCUUUUAUCACGUUUUAGAGCUAGAAAUAGCAAGUUAAAAUAAGGCUAGUC |
| | CGUUAUCAACUUGAAAAAGUGGCACCGAGUCGGUGCUUCCUGGAGCCUGCCAUAAAAGCAACUGUU* |
| | mU*mU*mUU (SEQ ID NO: 22) |
| BCL11A_+58_v2 | mC*mU*mA*ACAGUUGCUUUUAUCACGUUUUAGAGCUAGAAAUAGCAAGUUAAAAUAAGGCUAGUC |
| | CGUUAUCAACUUGAAAAAGUGGCACCGAGUCGGUGCUUCCUGGAGGGUGCCAUAAAAGCAACUGUU* |
| | mU*mU*mUU (SEQ ID NO: 23) |
| Niking sgRNA | |
| name | Sequence |
| ATPIAI-G3 | mG*mA*mG*UUCUGUAAUUCAGCAUAGUUUUAGAGCUAGAAAUAGCAAGUUAAAAUAAGGCUAGUC |
| | CGUUAUCAACUUGAAAAAGUGGCACCGAGUCGGUGCmU*mU*mU*U (SEQ ID NO: 24) |
| HBB Nick 1 | mC*mC*mU*UGAUACCAACCUGCCCAGUUUUAGAGCUAGAAAUAGCAAGUUAAAAUAAGGCUAGUC |
| | CGUUAUCAACUUGAAAAAGUGGCACCGAGUCGGUGCmU*mU*mU*U (SEQ ID NO: 25) |
| HBB Nick 2 | mG*mU*mA*ACGGCAGACUUCUCUUCGUUUUAGAGCUAGAAAUAGCAAGUUAAAAUAAGGCUAGUC |
| (PE3b) | CGUUAUCAACUUGAAAAAGUGGCACCGAGUCGGUGCmU*mU*mU*U (SEQ ID NO: 26) |
| B2M Nick | mA*mG*mU*GGAGGCGUCGCGCUGGCGUUUUAGAGCUAGAAAUAGCAAGUUAAAAUAAGGCUAGUC |
| | CGUUAUCAACUUGAAAAAGUGGCACCGAGUCGGUGCmU*mU*mU*U (SEQ ID NO: 27) |
| RNF2 Nick | mT*mC*mA*ACCATTAAGCAAAACATGUUUUAGAGCUAGAAAUAGCAAGUUAAAAUAAGGCUAGUCC |
| | GUUAUCAACUUGAAAAAGUGGCACCGAGUCGGUGCmU*mU*mU*U (SEQ ID NO: 28) |
| HBG1/2 Nick | mG*mG*mA*AUGACUGAAUCGGAACAGUUUUAGAGCUAGAAAUAGCAAGUUAAAAUAAGGCUAGUC |
| | CGUUAUCAACUUGAAAAAGUGGCACCGAGUCGGUGCmU*mU*mU*U (SEQ ID NO: 29) |

Phosphorothioate linkage are highlighted with *
2'-O-Methyl modified bases are highlighted with a lowercase m

### Genome Editing Vectors

Prime editing experiments in K562 and Jurkat cell lines were performed with pCMV-PEmax (Addgene 174820). The ATP1A1-Q118R_v2 epegRNA was cloned into pU6-tevopreq1-GG-acceptor (Addgene 174038) and the ATP LA1-G3 nicking sgRNA was cloned into SpCas9_sgRNA_expression_in_pBluescript (Addgene 122089). For SIV-Vpx, SAMHD1, and the inactive SAMHD1-H215A variant overexpression under the transcriptional control of the CMV promoter, the PEmax cassette from pCMV-PEmax (Addgene 174820) was swapped with the indicated cassette.

### Nucleofection

For standard conditions, CD34⁺ HSPCs were thawed and cultured for 24 hours in the presence of cytokines prior to nucleofection. CD34⁺ HSPCs were electroporated using the P3 Primary Cell X kit S (Lonza) according to manufacturer's recommendations. Unless otherwise indicated, 2.5×10⁵ cells were electroporated with 2000 ng PE mRNA, 200 pmol (e) pegRNA, and 100 pmol nicking sgRNA using pulse code DS-130. For twin prime editing conditions, 2.5×10⁵ cells were electroporated with 2000 ng PEmax mRNA and 150 pmol of each (e) pegRNA using pulse code DS-130. For Vpx mRNA co-delivery, Vpx: PEmax mRNAs were electroporated at an equimolar ratio. For quiescent HSPCs nucleofections, HSPCs where thawed and 5×10⁵ cells were electroporated without prior culture with cytokines. Following electroporation, 80 μl of media supplemented with cytokines was added to each well and cells were incubated for 10 minutes prior to transfer to the culture plate. Where indicated, cells from one nucleofection were split in two wells in a 96-well plate with or without the indicated concentration of each deoxynucleosides. For Vpx VLPs experiments, the culture media was supplemented with 1× Lentiboost®. DMEM supplemented with 10% FBS and 1% Penicillin/Streptomycin was used as a vehicle control for Vpx VLPs from the GPP platform. CD34⁺ HSPCs were cultured for 72 hours before genotyping.

K562 cells (2×10⁵ cells) were electroporated with 750 ng PEmax vector, 250 ng epegRNA vector, and 100 ng nicking sgRNA vector using the SF nucleofection kit (Lonza) and pulse code FF-120. Jurkat cells (5×10⁵ cells) were electroporated with 500 ng PEmax vector, 250 ng epegRNA vector, and 100 ng nicking sgRNA vector using the SE nucleofection kit (Lonza) and pulse code CL-120. K562 and Jurkat cells were cultured for 72 hours before genotyping.

### Genotyping

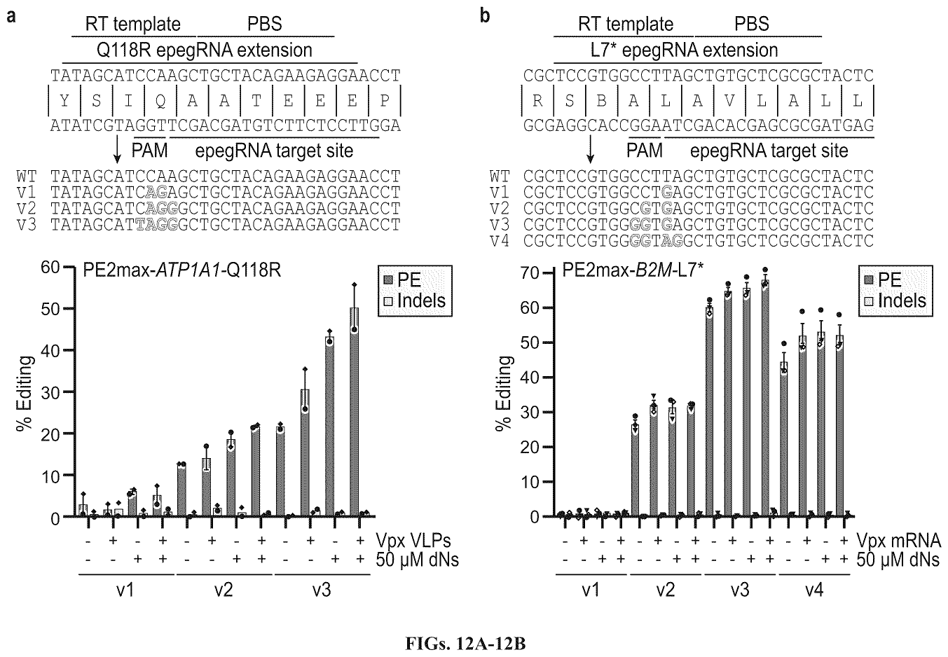

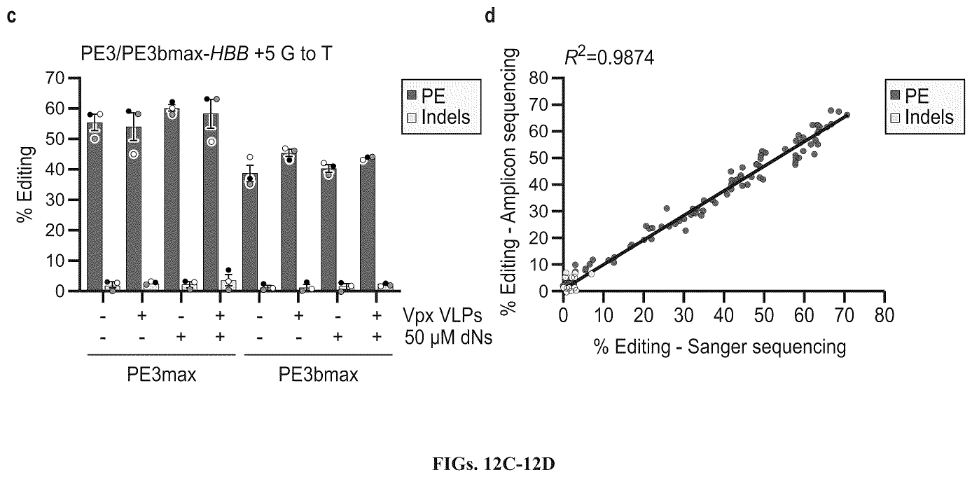

Three days post-nucleofection, cells were washed once with 500 μl PBS and genomic DNA was harvested using QuickExtract DNA extraction solution (Epicentre) following manufacturer's recommendations. For Sanger sequencing, PCR amplifications were performed with 30 cycles of amplification with Phusion high-fidelity polymerase. PCR product quality was evaluated by electrophoresis, and purification was performed using SPRI magnetic beads prior to Sanger sequencing. Sequencing traces quality was manually inspected using Geneious R11, and sequencing reactions with background noise were repeated. Sequencing traces with little to no background were used for quantification using BEAT (Xu et al., Cris. J. 2, 223-229 (2019)) for substitutions, and TIDE (Brinkman et al., Nucleic Acids Res. 42, e168 (2014)) for indels. A comparison between Sanger and amplicon sequencing was performed at three loci using PE2max, PE3max, and PE3bmax to validate quantification accuracy (FIG. 12). A strong correlation (R²=0.9874) between Sanger and amplicon sequencing was observed with 96 samples (FIG. 12D), corroborating previous observations (Brinkman et al. Nucleic Acids Res. 46, e58 (2018); Wu et al., Nat. Med. 25, 776-783 (2019); Bloh et al., Cris. J. 4, 120-131 (2021); Conant et al., Cris. J. 5, 123-130 (2022)).

For amplicon sequencing, PCR amplifications were performed with Phusion high-fidelity polymerase or KOD Hot Start DNA Polymerase and PCR product quality was evaluated by electrophoresis. One μl of locus-specific PCR product was used for indexing (PCR 2). Amplicon PCR products were purified using SPRI magnetic beads, and quality was evaluated by electrophoresis and TapeStation using D1000 high sensitivity Screen tape (Agilent). Amplicons were sequenced using paired-end 150 bp reads on an Illumina NovaSeq X system by Novogene (Durham, NC, USA) or in-house on an Illumina MiniSeq system. The percentage of prime edited or indels alleles were quantified using CRISPResso2 (Clement et al. Nat. Biotechnol. 37, 215-226 (2019)). The percentage of indel reads was determined as the percentage of modified reads and substitutions were excluded for the analysis. For twin prime editing, CRISPResso2 was run in HDR mode using the desired allele as the expected allele and the percentage of indels was determined as the % NHEJ reads+% Imperfect HDR reads, and substitutions were excluded for indels quantification. Primers used in this study and the amplicon sizes are provided in the Supplementary material section.

### Droplet Digital PCR

Directly after thawing, 5×10⁵ quiescent HSPCs were electroporated with 2000 ng PEmax mRNA and 150 pmol of AAVS1-A1615a epegRNA to synthesize a non-homologous 3′ ssDNA flap that does not integrate to its target locus, and cells were culture in the presence or absence of 50 μM each dNs. Genomic DNA was harvested 6 hours after electroporation, and genomic DNA was extracted and purified using the DNeasy Blood and Tissue Kit (Qiagen). 25-50 ng of genomic DNA was used for each ddPCR assay. The droplets were generated using a Bio-Rad QX200 AutoDG droplet digital PCR system with ddPCR supermix (no dUTP) (Bio-Rad), and HindIII-HF was supplemented (NEB) in each reaction. Following droplet generation, samples were amplified with the following conditions: 95° C. for 10 minutes, 40 cycles of 94° C. for 30 seconds and 60° C. for 60 seconds, and a final incubation at 98° C. for 10 minutes. Samples were then kept at 4° C. until analysis. Results were analyzed using the QuantaSoft software, and the percentage of alleles harboring the reverse transcribed 3′ DNA flap was determined as the ratio of flap allele relative to the genomic reference EIF2C1 refv3 (Bio-Rad). Primer and probe sequences are provided in Table 3, below.

TABLE 3
Primers and Probes Used

| | | |
|---|---|---|
| | | PCR |
| | | product |
| Primer | Sequence | size |

Primers used for Sanger sequencing

| | | |
|---|---|---|
| ATP1A1-Forward | TCTGGGTGTTATGAGTTCCTTGGG (SEQ ID NO: 30) | 740 bp |
| ATP1A1-Reverse | AACATGAATCGCTGTGCTCTGC (SEQ ID NO: 31) | |
| HBB-Forward | CTGAGGGTTTGAAGTCCAACTCC (SEQ ID NO: 32) | 760 bp |
| HBB-Reverse | CCTGTTACTTATCCCCTTCCTATGAC (SEQ ID NO: 33) | |
| B2M-Forward | CCTCCAGCCTGAAGTCCTAGAATG (SEQ ID NO: 34) | 713 bp |
| B2M-Reverse | GCGAAAGAGCGGAAGAGAAACC (SEQ ID NO: 35) | |
| RNF2-Forward | GCCAAAAGTTTCCATCAAGCCTC (SEQ ID NO: 36) | 665 bp |
| RNF2-Reverse | CAGTCTTCCTTGGTGCCTTATCAG (SEQ ID NO: 37) | |

Primers used for amplicon sequencing

| | | |
|---|---|---|
| ATP1A1-NGS-Forward | CTACACGACGCTCTTCCGATCTGGGGGTTCTCAATGTTACTGTGG (SEQ ID NO: 38) | 281 bp |
| ATP1A1-NGS-Reverse | AGACGTGTGCTCTTCCGATCTGAAGGCAACAGCTGTTCATAACC (SEQ ID NO: 39) | |
| HBB-NGS-Forward | CTACACGACGCTCTTCCGATCTAGGGCTGGGCATAAAAGTCAG (SEQ ID NO: 40) | 278 bp |
| HBB-NGS-Reverse | AGACGTGTGCTCTTCCGATCTTGCCCAGTTTCTATTGGTCTCC (SEQ ID NO: 41) | |
| AAVS1-NGS-Forward | CTACACGACGCTCTTCCGATCTCATTTCCTGGAGCCATCTCTCTC (SEQ ID NO: 42) | 283 bp |
| AAVS1-NGS-Reverse | AGACGTGTGCTCTTCCGATCTTCTTGGGAAGTGTAAGGAAGCTG (SEQ ID NO: 43) | |
| CCR5-NGS-Forward | CTACACGACGCTCTTCCGATCTCTTTAAAAGCCAGGACGGTCACC (SEQ ID NO: 44) | 283 bp |
| CCR5-NGS-Reverse | AGACGTGTGCTCTTCCGATCTCCGAGTAGCAGATGACCATGACA (SEQ ID NO: 45) | |
| B2M-NGS-Forward | CTACACGACGCTCTTCCGATCTCGCGTTTAATATAAGTGGAGGC (SEQ ID NO: 46) | 284 bp |
| B2M-NGS-Reverse | AGACGTGTGCTCTTCCGATCTGGAGAACTTGGAGAAGGGAAGT (SEQ ID NO: 47) | |
| HBG1/2-NGS-Forward | CTACACGACGCTCTTCCGATCTCGGCTGACAAAAGAAGTCCTGG (SEQ ID NO: 48) | 285 bp |
| HBG1/2-NGS-Reverse | AGACGTGTGCTCTTCCGATCTCTATTGGTCAAGGCAAGGCTGG (SEQ ID NO: 49) | |
| BCL11A-NGS-Forward | CTACACGACGCTCTTCCGATCTAGAGAGCCTTCCGAAAGAGG (SEQ ID NO: 50) | 263 bp |
| BCL11A-NGS-Reverse | AGACGTGTGCTCTTCCGATCTGGCCAGAAAAGAGATATGGCATC (SEQ ID NO: 51) | |

Primers and probes used for droplet digital PCR

| | | |
|---|---|---|
| AAVS1-A1615a_Flap_Forward | TACCGTACACCACTGAGACC (SEQ ID NO: 52) | 125 bp |
| AAVS1-A1615a_Flap_Reverse | TCTTGGGAAGTGTAAGGAAGC (SEQ ID NO: 53) | |
| AAVS1-A1615a_Flap_Probe | CTCCCAGAACCTGAGCTGCTCTG (SEQ ID NO: 54) | |
| EIF2C1_Refv3_Forward | CTAGCCATTGTGAGCTGGC (SEQ ID NO: 55) | 125 bp |
| EIF2C1 Refv3_Reverse | ACCCAATACCTCATGGATGC (SEQ ID NO: 56) | |
| EIF2C1_Refv3_Probe | CCCTGGATGTGGCCATGAGG (SEQ ID NO: 57) | |

### Exemplary Sequences Used

VSV-G plasmid (Addgene #12259)
(SEQ ID NO: 58)
TTTAAATACATCATTGCAATGAAAATAAATGTTTTTTATTAGGCAGAATCCAGAT
GCTCAAGGCCCTTCATAATATCCCCCAGTTTAGTAGTTGGACTTAGGGAACAAAG
GAACCTTTAATAGAAATTGGACAGCAAGAAAGCGAGCTTAGTGATACTTGTGGG
CCAGGGCATTAGCCACACCAGCCACCACTTTCTGATAGGCAGCCTGCACTGGTGG
GGTGAATTCCGTTTTTTTTTTTTTTTTTCATAAAAATTAAAAACTCAAATATAATT
GAGGCCTCTTTGAGCATGGTATCACAAGTTGATTTGGTCCAAACATGAAGAATCT
GTTGTGCAGGATTTGAGTTACTTTCCAAGTCGGTTCATCTCTATGTCTGTATAAAT
CTGTCTTTTCTTGGTGTGCTTTAATTTAATGCAAAGATGGATACCAACTCGGAGA
ACCAAGAATAGTCCAATGATTAACCCTATGATAAAGAAAAAAGAGGCAATAGAG
CTTTTCCAACTACTGAACCAACCTTCTACAAGCTCGATTGGATTTTTGGATAGCCC
AGTATCACCAAAAAATAAACTCTCATCATCAGGAAGTTGCGAAGCAGCGTCTTG
AATGTGAGGATGTTCGAACACCTGAGCCTTTGAGCTAAGATGAAGATCGGAGTC
CAACATACCATGTCCAATCATGTATAAAGGAAACTTATATCCTGAACTGGTCCTC
AGAACTCCATTGGGTCCAATTTCCACGTCTTCATATGGTGCCCAGTCATCCCACA
GTTCCCTTTCTGTGGTAGTTCCACTGATCATTCCGACCATTCTTGAGAGGATTGGA
GCAGCAATATCGACTCTGATGTATCTGGTCTCAAAGTATTTTAGGGTACCATTGA
TTATGGTGAAAGCAGGACCGGTTCCTGGGTTTTTAGGAGCAAGATAGCTGAGATC
CACTGGAGAGATTGGAAGACCCGCTCTGATTTTGCTCCAGGTTTCTTGGCAGAGG
GAATAATCCAAGATCCTCTCAACGTCCTGAATTAGACTTACATCCACTGAGGTCT
GAGATGGAGCAGAGATACTTGACCCTTCTGGGCATTCAGGGAATCTGGCTGCAG
CAAAGAGATCCTTATCAGCCATCTCGAACCAGACACCTGATGGGAGTCTGACTCC
CCAATGCTTGCAGTATTGCATTTTGCAGGCCTTGCCTCCAGTTTCATAAGCAAAG
TAGTTACTTCTGAACCCTGTGCCCTCCTTTCCCAGGGATGATAGCTCTCCGTCCTC
TGAGAAGAAGGTGATGTCCATGGAAATGAGGTTAGAATCACATAGCCCTTTGAC
CTTATAGTCAGAATGCCAGGTTGTAGAGTTATGGACAGTGGGGCATATGTAATTG
CTGCATTTTCCGTTGATGAACTGTGAATCAACCCATTCTCCTGTGTATTCATCAAC
CAGCACATGGTGAGGAGTCACCTGGACAATCACTGCTTCGGCATCCGTCACAGTT
GCATATCCACAACTTTGAGGAGGGAAGCCTGGATTCAGCCAAGTTCCTTGTTTCG
TTTGTTCAATGCTTTCCTTGCATTGTTCTACAGATGGAGTGAAGGATCGGATGGA
ATGTGTTATATACTTCGGTCCATACCAGCGGAAATCACAAGTAGTGACCCATTTG
GAAGCATGACACATCCAACCGTCTGCTTGAATAGCCTTGTGACTCTTGGGCATTT
TGACTTGTAAGGCTGTGCCTATTAAGTCATTATGCCAATTTAAATCTGAGCTTGA
CGGGCAATAATGGTAATTAGAAGGAACATTTTTCCAGTTTCCTTTTTGGTTGTGTG
GAAAAACTATGGTGAACTTGCAATTCACCCCAATGAATAAAAAGGCTAAGTACA
AAAGGCACTTCATAGTGTCAGAATTCAGATCTCACGTGCTTTGCCAAAGTGATGG
GCCAGCACACAGACCAGCACGTTGCCCAGGAGCTGTGGGAGGAAGATAAGAGG
TATGAACATGATTAGCAAAAGGGCCTAGCTTGGACTCAGAATAATCCAGCCTTAT
CCCAACCATAAAATAAAAGCAGAATGGTAGCTGGATTGTAGCTGCTATTAGCAA
TATGAAACCTCTTACATCAGTTACAATTTATATGCAGAAATATTTATATGCAGAA
ATATTGCTATTGCCTTAACCCAGAAATTATCACTGTTATTCTTTAGAATGGTGCAA
AGAGGCATGATACATTGTATCATTATTGCCCTGAAAGAAAGAGATTAGGGAAAG
TATTAGAAATAAGATAAACAAAAAAGTATATTAAAAGAAGAAAGCATTTTTTAA
AATTACAAATGCAAAATTACCCTGATTTGGTCAATATGTGTACCCTGTTACTTCTC
CCCTTCCTATGACATGAACTTAACCATAGAAAAGAAGGGGAAAGAAAACATCAA
GGGTCCCATAGACTCACCCTGAAGTTCTCAGGATCCGAGCTCGGTACCACATGTA
AGCTTCGAGGGGAGGCTGGATCGGTCCCGGTGTCTTCTATGGAGGTCAAAACAG
CGTGGATGGCGTCTCCAGGCGATCTGACGGTTCACTAAACGAGCTCTGCTTATAT
AGACCTCCCACCGTACACGCCTACCGCCCATTTGCGTCAATGGGGCGGAGTTGTT
ACGACATTTTGGAAAGTCCCGTTGATTTTGGTGCCAAAACAAACTCCCATTGACG
TCAATGGGGTGGAGACTTGGAAATCCCCGTGAGTCAAACCGCTATCCACGCCCA
TTGATGTACTGCCAAAACCGCATCACCATGGTAATAGCGATGACTAATACGTAG
ATGTACTGCCAAGTAGGAAAGTCCCATAAGGTCATGTACTGGGCATAATGCCAG
GCGGGCCATTTACCGTCATTGACGTCAATAGGGGGCGTACTTGGCATATGATACA
CTTGATGTACTGCCAAGTGGGCAGTTTACCGTAAATACTCCACCCATTGACGTCA
ATGGAAAGTCCCTATTGGCGTTACTATGGGAACATACGTCATTATTGACGTCAAT
GGGCGGGGGTCGTTGGGCGGTCAGCCAGGCGGGCCATTTACCGTAAGTTATGTA
ACGCGGAACTCCATATATGGGCTATGAACTAATGACCCCGTAATTGATTACTATT
AATAACTAGTCAATAATCAATGTCAACATGGCGGTAATGTTGGACATGAGCCAA
TATAAATGTACATATTATGATATGGATACAACGTATGCAATGGGCCAAGCTCATG
GCTGACTAATTTTTTTTATTTATGCAGAGGCCGAGGCCGGATCCTCTAGCCCATG
GGGGCCCCCTCAGGGGATCCACGTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTGATTCTGT
GGATAACCGTATTACCGCCTTTGAGTGAGCTGATACCGCTCGCCGCAGCCGAACG
ACCGAGCGCAGCGAGTCAGTGAGCGAGGAAGCAGATCTGCGGCCGCACTAGTGC
TAGACTGCCATGTCGAGGGATTCCGGGTCACTGTGAGTGGGGGAGGCAGGGAAG
AAGGGCTCACAGGACAGTCAAACCATGCCCCCTGTTTTTCCTTCTTCAAGTAGAC
CTCTATAAGACAACAGAGACAACTAAGGCTGAGTGGCCAGGCGAGGAGAAACC
ATCTCGCCGTAAAACATGGAAGGAACACTTCAGGGGAAAGGTGGTATCTCTAAG
CAAGAGAACTGAGTGGAGTCAAGGCTGAGAGATGCAGGATAAGCAAATGGGTA
GTGAAAAGACATTCATGAGGACAGCTAAAACAATAAGTAATGTAAAATACAGCA
TAGCAAAACTTTAACCTCCAAATCAAGCCTCTACTTGAATCCTTTTCTGAGGGAT
GAATAAGGCATAGGCATCAGGGGCTGTTGCCAATGTGCATTAGCTGTTTGCAGCC
TCACCTTCTTTCATGGAGTTTAAGATATAGTGTATTTTCCCAAGGTTTGAACTAGC
TCTTCATTTCTTTATGTTTTAAATGCACTGACCTCCCACATTCCCTTTTTAGTAAAA
TATTCAGAAATAA
VPX (SIV)
(SEQ ID NO: 3)
ATGTCAGATCCCAGGGAGAGAATCCCACCTGGAAACAGTGGAGAAGAGACAAT
AGGAGAGGCCTTCGAATGGCTAAACAGAACAGTAGAGGAGATAAACAGAGAGG
CAGTAAACCACCTACCAAGGGAGCTGATTTTCCAGGTTTGGCAAAGGTCTTGGG
AATACTGGCATGATGAACAAGGGATGTCACAAAGCTATGTAAAATACAGATACT
TGTGTTTAATGCAAAAGGCTTTATTTATGCATTGCAAGAAAGGCTGTAGATGTCT
AGGGGAAGGACACGGGGCAGGAGGATGGAGACCAGGACCTCCTCCTCCTCCCCC
TCCAGGACTAGCATGA
VPX (HIV2)
(SEQ ID NO: 4)
ATGACAGACCCCAGAGAAAGGGTACCGCCAGGAAACAGTGGAGAAGAGACCAT
TGGAGAGGCCTTCGAGTGGCTAGAGAGGACCATAGAAGCCTTAAACAGGGAGGC
AGTGAACCATCTGCCCCGAGAGCTCATTTTCCAGGTGTGGCAAAGGTCCTGGAG
ATATTGGCATGATGAACAAGGGATGTCAGCAAGCTACACAAAGTATAGATATTT
GTGCCTAATGCAAAAAGCTATATTTACACATTTCAAGAGAGGGTGCACTTGCTGG
GGGGAGGACATGGGCCGGGAAGGATTGGAAGACCAAGGACCTCCCCCTCCTCCC
CCTCCAGGTCTAGTCTAA
VPX (SIV) VLPs: Complete coding sequence of POL, VIF,
VPX, and VPR from pSIV VPX003
(SEQ ID NO: 59)
GTGTTGGAATTGTGGGAAGGAGGGACACTCTGCAAGGCAATGCAGAGCCCCAAG
AAGACAGGGATGCTGGAAATGTGGAAAAATGGACCATGTTATGGCCAAATGCCC
AGACAGACAGGCGGGTTTTTTAGGCCTTGGTCCATGGGGAAAGAAGCCCCGCAA
TTTCCCCATGGCTCAAGTGCATCAGGGGCTGACGCCAACTGCTCCCCCAGAGGAC
CCAGCTGTGGATCTGCTAAAGAACTACATGCAGTTGGGCAAGCAGCAGAGAGAA
AGCAGAGAGAAGCCTTACAAGGAGGTGACAGAGGATTTGCTGCACCTCAATTCT
CTCTTTGGAGGAGACCAGTAGTCACTGCTCATATTGAAGGACAGCCTGTAGAAGT
ATTATTGGATACAGGGGCTGATGATTCTATTGTAACAGGAATAGAGTTAGGTCCA
CATTATACCCCAAAAATAGTAGGAGGAATAGGAGGTTTTATTAATACTAAAGAA
TACAAAAATGTAAAAATAGAAGTTTTAGGCAAAAGGATTAAAGGGACAATCATG
ACAGGGGACACTCCGATTAACATTTTTGGTAGGAATTTGCTAACAGCTCTGGGGA
TGTCTCTAAATCTTCCCATAGCTAAGGTAGAGCCTGTAAAAGTCACCTTAAAGCC
AGGAAAGGTTGGACCAAAATTGAAGCAGTGGCCATTATCAAAAGAAAAGATAGT
TGCATTAAGAGAAATCTGTGAAAAGATGGAAAAGGATGGTCAGTTGGAGGAAGC
TCCCCCGACCAATCCATACAACACCCCCACATTTGCCATAAAGAAAAAAGATAA
GAACAAATGGAGAATGCTGATAGATTTTAGGGAACTAAATAGGGTCACTCAGGA
CTTTACAGAAGTCCAATTAGGAATACCACACCCTGCAGGACTAGCAAAAAGGAA
AAGGATTACAGTACTGGATATAGGTGATGCATATTTCTCCATACCTCTAGATGAA
GAATTTAGGCAGTACACTGCCTTTACTTTACCATCAGTAAATAATGCAGAGCCAG
GAAAACGATACATTTATAAGGTTCTGCCTCAGGGATGGAAGGGGTCACCAGCCA
TCTTCCAATACACTATGAGACATGTGCTAGAACCCTTCAGGAAGGCAAATCCAG
ATGTGACCTTAGTCCAGTATATGGATGACATCTTAATAGCTAGTGACAGGACAGA
CCTGGAACATGACAGGGTAGTTTTACAGCTAAAGGAACTCTTAAATAGCATAGG
GTTCTCTACCCCAGAAGAGAAATTCCAAAAAGATCCCCCATTTCAATGGATGGG
GTACGAATTGTGGCCGACAAAATGGAAGTTGCAAAAGATAGAGTTGCCACAAAG
AGAGACCTGGACAGTGAATGATATACAGAAGTTAGTAGGAGTATTAAATTGGGC
AGCTCAAATTTATCCAGGTATAAAAACCAAACATCTCTGTAGGTTAATTAGAGGA
AAAATGACTCTAACAGAGGAAGTTCAGTGGACTGAGATGGCAGAAGCAGAATAT
GAGGAAAATAAGATAATTCTCAGTCAGGAACAAGAAGGATGTTATTACCAAGAA
GGCAAGCCATTAGAAGCCACGGTAATAAAGAGTCAGGACAATCAGTGGTCTTAT
AAAATTCACCAAGAAGACAAAATACTGAAAGTAGGAAAATTTGCAAAGATAAA
GAATACACATACCAATGGAGTTAGACTATTAGCACATGTAATACAGAAAATAGG
AAAGGAAGCAATAGTGATCTGGGGACAGGTCCCAAAATTCCACTTACCAGTTGA
GAGGGATGTATGGGAACAGTGGTGGACAGACTATTGGCAGGTAACCTGGATACC
GGAGTGGGATTTTATCTCAACGCCACCACTAGTAAGATTAGTCTTCAATCTAGTG
AAGGACCCTATAGAGGGAGAAGAAACCTATTATACAGATGGATCATGTAATAAA
CAGTCAAAAGAAGGGAAAGCAGGATATATCACAGATAGGGGCAAAGACAAAGT
AAAAGTGTTAGAACAGACTACTAATCAACAAGCAGAATTAGAAGCATTTCTCAT
GGCATTGACAGACTCAGGGCCAAAGACAAATATTATAGTAGATTCACAATATGT
TATGGGAATAATAACAGGATGCCCTACAGAATCAGAGAGCAGGCTAGTTAACCA
AATAATAGAAGAAATGATTAAAAAGTCAGAAATTTATGTAGCATGGGTACCAGC
ACACAAAGGTATAGGAGGAAACCAAGAAATAGACCACCTAGTTAGTCAGGGGA
TTAGACAAGTTCTCTTCTTGGAAAAGATAGAGCCAGCACAAGAAGAACATGATA
AATACCATAGTAATGTAAAAGAATTGGTATTCAAATTTGGATTACCCAGAATAGT
GGCCAGACAGATAGTAGACACCTGTGATAAATGTCATCAGAAAGGAGAAGCTAT
ACATGGGCAGGTAAATTCAGATCTAGGGACTTGGCAAATGGACTGTACCCATCT
AGAAGGAAAAATAGTCATAGTTGCAGTACATGTAGCTAGTGGATTCATAGAAGC
AGAAGTAATTCCACAAGAGACAGGAAGACAGACAGCACTATTTCTGTTAAAATT
GGCAGGCAGATGGCCTATTACACATCTACACACAGATAATGGTGCTAACTTTGCC
TCGCAAGAAGTAAAGATGGTTGCATGGTGGGCAGGGATAGAGCACACCTTTGGG
GTACCATACAATCCACAGAGTCAGGGAGTAGTGGAAGCAATGAATCACCACCTG
AAAAATCAAATAGATAGAATCAGGGAACAAGCAAATTCAGTAGAAACCATAGT
ATTAATGGCAGTTCATTGCATGAATTTTAAAAGAAGGGGAGGAATAGGGGATAT
GACTCCAGCAGAAAGATTAATTAACATGATCACTACAGAACAAGAAATACAATT
TCAACAATCAAAAAACTCAAAATTTAAAAATTTTCGGGTCTATTACAGAGAAGG
CAGAGATCAACTGTGGAAGGGACCCGGTGAGCTATTGTGGAAAGGGGAAGGAG
CAGTCATCTTAAAGGTAGGGACAGACATTAAGGTAGTACCCAGAAGAAAGGCTA
AAATTATCAAAGATTATGGAGGAGGAAAAGAGGTGGATAGCAGTTCCCACATGG
AGGATACCGGAGAGGCTAGAGAGGTGGCATAGCCTCATAAAATATCTGAAATAT
AAAACTAAAGATCTACAAAAGGTTTGCTATGTGCCCCATTTTAAGGTCGGATGGG
CATGGTGGACCTGCAGCAGAGTAATCTTCCCCCTACAGGAAGGAAGCCATTTAG
AAGTACAAGGGTATTGGCATTTGACACCAGAAAGAGGGTGGCTCAGTACTTATG
CAGTGAGGATAACATGGTACTCAAGGAACTTTTGGACAGATGTAACACCAGACT
ATGCAGACATTTTACTGCATAGCACTTATTTCCCTTGCTTTACAGCGGGAGAAGT
GAGAAGGGCCATCAGGGGAGAACAACTGCTGTCTTGCTGCAAGTTCCCGAGAGC
ACATAGGTACCAGGTACCAAGCCTACAGTACTTAGCACTAAAAGTAGTAAGCGA
TGTCAGATCCCAGGGAGAGAATCCCACCTGGAAACAGTGGAGAAGAGACAATA
GGAGAGGCCTTCGAATGGCTAAACAGAACAGTAGAGGAGATAAACAGAGAGGC
AGTAAACCACCTACCAAGGGAGCTGATTTTCCAGGTTTGGCAAAGGTCTTGGGA
ATACTGGCATGATGAACAAGGGATGTCACAAAGCTATGTAAAATACAGATACTT
GTGTTTAATGCAAAAGGCTTTATTTATGCATTGCAAGAAAGGCTGTAGATGTCTA
GGGGAAGGACACGGGGCAGGAGGATGGAGACCAGGACCTCCTCCTCCTCCCCCT
CCAGGACTAGCATAAATGGAAGAAAGACCTCCGGAAAATGAAGGCCCACAAAG
GGAACCATGGGATGAATGGGTAGTGGAGGTTCTGGAAGAATTGAAAGAAGAAG
CTTTAAAACATTTTGATCCTCGCTTGCTAACTGCACTTGGTAATCATATCTATAAT
AGACATGGAGACACCCTTGAGGGAGCAGGAGAACTCATTAGAATCCTCCAACGA
GCGCTCTTCATGCATTTTAGAGGCGGATGCAACCACTCCAGAATCGGCCAACCTG
GGGGAGGAAATCCTCTCTCAACTATACCGCCCTCTTGA
epegRNA and nick sgRNA
ATPIA1-Q118R_v2 epegRNA¹
(SEQ ID NO: 60)
GUUCCUCUUCUGUAGCAGCUGUUUUAGAGCUAGAAAUAGCAAGUUAAAAUAA
GGCUAGUCCGUUAUCAACUUGAAAAAGUGGCACCGAGUCGGUGCUAGCAUCA
GGGCUGCUACAGAAGAGCGCGGUUCUAUCUAGUUACGCGUUAAACCAACUAG
AAUUU
ATP1A1-G3 nick sgRNA¹
(SEQ ID NO: 61)
GAGUUCUGUAAUUCAGCAUAGUUUUAGAGCUAGAAAUAGCAAGUUAAAAUAA
GGCUAGUCCGUUAUCAACUUGAAAAAGUGGCACCGAGUCGGUGCUUUU
HBB-E6V correction epegRNA²
(SEQ ID NO: 62)
CAUGGUGCACCUGACUCCUGGUUUUAGAGCUAGAAAUAGCAAGUUAAAAUAA
GGCUAGUCCGUUAUCAACUUGAAAAAGUGGCACCGAGUCGGUGCAGACUUCUC
UUCAGGAGUCAGGUGCACAGAAUAAACGCGGUUCUAUCUAGUUACGCGUUAA
ACCAACUAGAAUUU
HBB-E6V PE3 nick sgRNA²
(SEQ ID NO: 63)
CCUUGAUACCAACCUGCCCAGUUUUAGAGCUAGAAAUAGCAAGUUAAAAUAA
GGCUAGUCCGUUAUCAACUUGAAAAAGUGGCACCGAGUCGGUGCUUUU
HBB-E6V PE3b nick sgRNA²
(SEQ ID NO: 64)
GUAACGGCAGACUUCUCUUCGUUUUAGAGCUAGAAAUAGCAAGUUAAAAUAA
GGCUAGUCCGUUAUCAACUUGAAAAAGUGGCACCGAGUCGGUGCUUUU
CCR5-Delta32 epegRNA³
(SEQ ID NO: 65)
AGAUGACUAUCUUUAAUGUCGUUUUAGAGCUAGAAAUAGCAAGUUAAAAUAA
GGCUAGUCCGUUAUCAACUUGAAAAAGUGGCACCGAGUCGGUGCAUUACACCU
GCAGCUCUCAUUUUCCUUAUAUUAAAGAUAGUCAUCCCACCAUACGCGGUUCU
AUCUAGUUACGCGUUAAACCAACUAGAAUUU
CCR5-Delta32 nick sgRNA³
(SEQ ID NO: 66)
CAGGACGGUCACCUUUGGGGGUUUUAGAGCUAGAAAUAGCAAGUUAAAAUAA
GGCUAGUCCGUUAUCAACUUGAAAAAGUGGCACCGAGUCGGUGCUUUU

### References for epegRNAs

1. Levesque, S. et al. Marker-free coselection for successive rounds of prime editing in human cells. Nat. Commun. 13, 5909 (2022).
2. Everette, K. A. et al. Ex vivo prime editing of patient haematopoietic stem cells rescues sickle-cell disease phenotypes after engraftment in mice. Nat. Biomed. Eng. (2023) doi: 10.1038/s41551-023-01026-0.
3. Liu, P. et al. Improved prime editors enable pathogenic allele correction and cancer modelling in adult mice. Nat. Commun. 12, 2121 (2021).

PE2
(SEQ ID NO: 67)
ATGAAACGGACAGCCGACGGAAGCGAGTTCGAGTCACCAAAGAAGAAGCGGAA
AGTCGACAAGAAGTACAGCATCGGCCTGGACATCGGCACCAACTCTGTGGGCTG
GGCCGTGATCACCGACGAGTACAAGGTGCCCAGCAAGAAATTCAAGGTGCTGGG
CAACACCGACCGGCACAGCATCAAGAAGAACCTGATCGGAGCCCTGCTGTTCGA
CAGCGGCGAAACAGCCGAGGCCACCCGGCTGAAGAGAACCGCCAGAAGAAGAT
ACACCAGACGGAAGAACCGGATCTGCTATCTGCAAGAGATCTTCAGCAACGAGA
TGGCCAAGGTGGACGACAGCTTCTTCCACAGACTGGAAGAGTCCTTCCTGGTGG
AAGAGGATAAGAAGCACGAGCGGCACCCCATCTTCGGCAACATCGTGGACGAGG
TGGCCTACCACGAGAAGTACCCCACCATCTACCACCTGAGAAAGAAACTGGTGG
ACAGCACCGACAAGGCCGACCTGCGGCTGATCTATCTGGCCCTGGCCCACATGA
TCAAGTTCCGGGGCCACTTCCTGATCGAGGGCGACCTGAACCCCGACAACAGCG
ACGTGGACAAGCTGTTCATCCAGCTGGTGCAGACCTACAACCAGCTGTTCGAGG
AAAACCCCATCAACGCCAGCGGCGTGGACGCCAAGGCCATCCTGTCTGCCAGAC
TGAGCAAGAGCAGACGGCTGGAAAATCTGATCGCCCAGCTGCCCGGCGAGAAGA
AGAATGGCCTGTTCGGAAACCTGATTGCCCTGAGCCTGGGCCTGACCCCCAACTT
CAAGAGCAACTTCGACCTGGCCGAGGATGCCAAACTGCAGCTGAGCAAGGACAC
CTACGACGACGACCTGGACAACCTGCTGGCCCAGATCGGCGACCAGTACGCCGA
CCTGTTTCTGGCCGCCAAGAACCTGTCCGACGCCATCCTGCTGAGCGACATCCTG
AGAGTGAACACCGAGATCACCAAGGCCCCCCTGAGCGCCTCTATGATCAAGAGA
TACGACGAGCACCACCAGGACCTGACCCTGCTGAAAGCTCTCGTGCGGCAGCAG
CTGCCTGAGAAGTACAAAGAGATTTTCTTCGACCAGAGCAAGAACGGCTACGCC
GGCTACATTGACGGCGGAGCCAGCCAGGAAGAGTTCTACAAGTTCATCAAGCCC
ATCCTGGAAAAGATGGACGGCACCGAGGAACTGCTCGTGAAGCTGAACAGAGA
GGACCTGCTGCGGAAGCAGCGGACCTTCGACAACGGCAGCATCCCCCACCAGAT
CCACCTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCCTAC
TACGTGGGCCCTCTGGCCAGGGGAAACAGCAGATTCGCCTGGATGACCAGAAAG
AGCGAGGAAACCATCACCCCCTGGAACTTCGAGGAAGTGGTGGACAAGGGCGCT
TCCGCCCAGAGCTTCATCGAGCGGATGACCAACTTCGATAAGAACCTGCCCAAC
GAGAAGGTGCTGCCCAAGCACAGCCTGCTGTACGAGTACTTCACCGTGTATAAC
GAGCTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGGAAA
GTGACCGTGAAGCAGCTGAAAGAGGACTACTTCAAGAAAATCGAGTGCTTCGAC
TCCGTGGAAATCTCCGGCGTGGAAGATCGGTTCAACGCCTCCCTGGGCACATACC
ACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGA
TGATCGAGGAACGGCTGAAAACCTATGCCCACCTGTTCGACGACAAAGTGATGA
AGCAGCTGAAGCGGCGGAGATACACCGGCTGGGGCAGGCTGAGCCGGAAGCTG
ATCAACGGCATCCGGGACAAGCAGTCCGGCAAGACAATCCTGGATTTCCTGAAG
TCCGACGGCTTCGCCAACAGAAACTTCATGCAGCTGATCCACGACGACAGCCTG
ACCTTTAAAGAGGACATCCAGAAAGCCCAGGTGTCCGGCCAGGGCGATAGCCTG
CACGAGCACATTGCCAATCTGGCCGGCAGCCCCGCCATTAAGAAGGGCATCCTG
CAGACAGTGAAGGTGGTGGACGAGCTCGTGAAAGTGATGGGCCGGCACAAGCCC
GAGAACATCGTGATCGAAATGGCCAGAGAGAACCAGACCACCCAGAAGGGACA
GAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAG
AAGCTGTACCTGTACTACCTGCAGAATGGGGGGATATGTACGTGGACCAGGAA
CTGGACATCAACCGGCTGTCCGACTACGATGTGGACGCTATCGTGCCTCAGAGCT
TTCTGAAGGACGACTCCATCGACAACAAGGTGCTGACCAGAAGCGACAAGAACC
GGGGCAAGAGCGACAACGTGCCCTCCGAAGAGGTCGTGAAGAAGATGAAGAAC
TACTGGCGGCAGCTGCTGAACGCCAAGCTGATTACCCAGAGAAAGTTCGACAAT
CTGACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCCTG
GACTCCCGGATGAACACTAAGTACGACGAGAATGACAAGCTGATCCGGGAAGTG
AAAGTGATCACCCTGAAGTCCAAGCTGGTGTCCGATTTCCGGAAGGATTTCCAGT
TTTACAAAGTGCGCGAGATCAACAACTACCACCACGCCCACGACGCCTACCTGA
ACGCCGTCGTGGGAACCGCCCTGATCAAAAAGTACCCTAAGCTGGAAAGCGAGT
TCGTGTACGGCGACTACAAGGTGTACGACGTGCGGAAGATGATCGCCAAGAGCG
AGCAGGAAATCGGCAAGGCTACCGCCAAGTACTTCTTCTACAGCAACATCATGA
ACTTTTTCAAGACCGAGATTACCCTGGCCAACGGCGAGATCCGGAAGCGGCCTCT
GATCGAGACAAACGGCGAAACCGGGGAGATCGTGTGGGATAAGGGCCGGGATT
TTGCCACCGTGCGGAAAGTGCTGAGCATGCCCCAAGTGAATATCGTGAAAAAGA
CCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGG
GCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACA
AAGAAGTGAAAAAGGACCTGATCATCAAGCTGCCTAAGTACTCCCTGTTCGAGC
TGGAAAACGGCCGGAAGAGAATGCTGGCCTCTGCCGGCGAACTGCAGAAGGGA
AACGAACTGGCCCTGCCCTCCAAATATGTGAACTTCCTGTACCTGGCCAGCCACT
ATGAGAAGCTGAAGGGCTCCCCCGAGGATAATGAGCAGAAACAGCTGTTTGTGG
AACAGCACAAGCACTACCTGGACGAGATCATCGAGCAGATCAGCGAGTTCTCCA
AGAGAGTGATCCTGGCCGACGCTAATCTGGACAAAGTGCTGTCCGCCTACAACA
AGCACCGGGATAAGCCCATCAGAGAGCAGGCCGAGAATATCATCCACCTGTTTA
CCCTGACCAATCTGGGAGCCCCTGCCGCCTTCAAGTACTTTGACACCACCATCGA
CCGGAAGAGGTACACCAGCACCAAAGAGGTGCTGGACGCCACCCTGATCCACCA
GAGCATCACCGGCCTGTACGAGACACGGATCGACCTGTCTCAGCTGGGAGGTGA
CTCTGGAGGATCTAGCGGAGGATCCTCTGGCAGCGAGACACCAGGAACAAGCGA
GTCAGCAACACCAGAGAGCAGTGGCGGCAGCAGCGGCGGCAGCAGCACCCTAA
ATATAGAAGATGAGTATCGGCTACATGAGACCTCAAAAGAGCCAGATGTTTCTC
TAGGGTCCACATGGCTGTCTGATTTTCCTCAGGCCTGGGCGGAAACCGGGGGCAT
GGGACTGGCAGTTCGCCAAGCTCCTCTGATCATACCTCTGAAAGCAACCTCTACC
CCCGTGTCCATAAAACAATACCCCATGTCACAAGAAGCCAGACTGGGGATCAAG
CCCCACATACAGAGACTGTTGGACCAGGGAATACTGGTACCCTGCCAGTCCCCCT
GGAACACGCCCCTGCTACCCGTTAAGAAACCAGGGACTAATGATTATAGGCCTG
TCCAGGATCTGAGAGAAGTCAACAAGCGGGTGGAAGACATCCACCCCACCGTGC
CCAACCCTTACAACCTCTTGAGCGGGCTCCCACCGTCCCACCAGTGGTACACTGT
GCTTGATTTAAAGGATGCCTTTTTCTGCCTGAGACTCCACCCCACCAGTCAGCCT
CTCTTCGCCTTTGAGTGGAGAGATCCAGAGATGGGAATCTCAGGACAATTGACCT
GGACCAGACTCCCACAGGGTTTCAAAAACAGTCCCACCCTGTTTAATGAGGCACT
GCACAGAGACCTAGCAGACTTCCGGATCCAGCACCCAGACTTGATCCTGCTACA
GTACGTGGATGACTTACTGCTGGCCGCCACTTCTGAGCTAGACTGCCAACAAGGT
ACTCGGGCCCTGTTACAAACCCTAGGGAACCTCGGGTATCGGGCCTCGGCCAAG
AAAGCCCAAATTTGCCAGAAACAGGTCAAGTATCTGGGGTATCTTCTAAAAGAG
GGTCAGAGATGGCTGACTGAGGCCAGAAAAGAGACTGTGATGGGGCAGCCTACT
CCGAAGACCCCTCGACAACTAAGGGAGTTCCTAGGGAAGGCAGGCTTCTGTCGC
CTCTTCATCCCTGGGTTTGCAGAAATGGCAGCCCCCCTGTACCCTCTCACCAAAC
CGGGGACTCTGTTTAATTGGGGCCCAGACCAACAAAAGGCCTATCAAGAAATCA
AGCAAGCTCTTCTAACTGCCCCAGCCCTGGGGTTGCCAGATTTGACTAAGCCCTT
TGAACTCTTTGTCGACGAGAAGCAGGGCTACGCCAAAGGTGTCCTAACGCAAAA
ACTGGGACCTTGGCGTCGGCCGGTGGCCTACCTGTCCAAAAAGCTAGACCCAGT
AGCAGCTGGGTGGCCCCCTTGCCTACGGATGGTAGCAGCCATTGCCGTACTGACA
AAGGATGCAGGCAAGCTAACCATGGGACAGCCACTAGTCATTCTGGCCCCCCAT
GCAGTAGAGGCACTAGTCAAACAACCCCCCGACCGCTGGCTTTCCAACGCCCGG
ATGACTCACTATCAGGCCTTGCTTTTGGACACGGACCGGGTCCAGTTCGGACCGG
TGGTAGCCCTGAACCCGGCTACGCTGCTCCCACTGCCTGAGGAAGGGCTGCAAC
ACAACTGCCTTGATATCCTGGCCGAAGCCCACGGAACCCGACCCGACCTAACGG
ACCAGCCGCTCCCAGACGCCGACCACACCTGGTACACGGATGGAAGCAGTCTCT
TACAAGAGGGACAGCGTAAGGCGGGAGCTGCGGTGACCACCGAGACCGAGGTA
ATCTGGGCTAAAGCCCTGCCAGCCGGGACATCCGCTCAGCGGGCTGAACTGATA
GCACTCACCCAGGCCCTAAAGATGGCAGAAGGTAAGAAGCTAAATGTTTATACT
GATAGCCGTTATGCTTTTGCTACTGCCCATATCCATGGAGAAATATACAGAAGGC
GTGGGTGGCTCACATCAGAAGGCAAAGAGATCAAAAATAAAGACGAGATCTTGG
CCCTACTAAAAGCCCTCTTTCTGCCCAAAAGACTTAGCATAATCCATTGTCCAGG
ACATCAAAAGGGACACAGCGCCGAGGCTAGAGGCAACCGGATGGCTGACCAAG
CGGCCCGAAAGGCAGCCATCACAGAGACTCCAGACACCTCTACCCTCCTCATAG
AAAATTCATCACCCTCTGGCGGCTCAAAAAGAACCGCCGACGGCAGCGAATTCG
AGCCCAAGAAGAAGAGGAAAGTCTAA
PEmax
(SEQ ID NO: 68)
ATGAAACGGACAGCCGACGGAAGCGAGTTCGAGTCACCAAAGAAGAAGCGGAA
AGTCGACAAGAAGTACAGCATCGGCCTGGACATCGGCACCAACTCTGTGGGCTG
GGCCGTGATCACCGACGAGTACAAGGTGCCCAGCAAGAAATTCAAGGTGCTGGG
CAACACCGACCGGCACAGCATCAAGAAGAACCTGATCGGAGCCCTGCTGTTCGA
CAGCGGCGAAACAGCCGAGGCCACCCGGCTGAAGAGAACCGCCAGAAGAAGAT
ACACCAGACGGAAGAACCGGATCTGCTATCTGCAAGAGATCTTCAGCAACGAGA
TGGCCAAGGTGGACGACAGCTTCTTCCACAGACTGGAAGAGTCCTTCCTGGTGG
AAGAGGATAAGAAGCACGAGCGGCACCCCATCTTCGGCAACATCGTGGACGAGG
TGGCCTACCACGAGAAGTACCCCACCATCTACCACCTGAGAAAGAAACTGGTGG
ACAGCACCGACAAGGCCGACCTGCGGCTGATCTATCTGGCCCTGGCCCACATGA
TCAAGTTCCGGGGCCACTTCCTGATCGAGGGCGACCTGAACCCCGACAACAGCG
ACGTGGACAAGCTGTTCATCCAGCTGGTGCAGACCTACAACCAGCTGTTCGAGG
AAAACCCCATCAACGCCAGCGGCGTGGACGCCAAGGCCATCCTGTCTGCCAGAC
TGAGCAAGAGCAGAAAGCTGGAAAATCTGATCGCCCAGCTGCCCGGCGAGAAG
AAGAATGGCCTGTTCGGAAACCTGATTGCCCTGAGCCTGGGCCTGACCCCCAACT
TCAAGAGCAACTTCGACCTGGCCGAGGATGCCAAACTGCAGCTGAGCAAGGACA
CCTACGACGACGACCTGGACAACCTGCTGGCCCAGATCGGCGACCAGTACGCCG
ACCTGTTTCTGGCCGCCAAGAACCTGTCCGACGCCATCCTGCTGAGCGACATCCT
GAGAGTGAACACCGAGATCACCAAGGCCCCCCTGAGCGCCTCTATGATCAAGAG
ATACGACGAGCACCACCAGGACCTGACCCTGCTGAAAGCTCTCGTGCGGCAGCA
GCTGCCTGAGAAGTACAAAGAGATTTTCTTCGACCAGAGCAAGAACGGCTACGC
CGGCTACATTGACGGCGGAGCCAGCCAGGAAGAGTTCTACAAGTTCATCAAGCC
CATCCTGGAAAAGATGGACGGCACCGAGGAACTGCTCGTGAAGCTGAAGAGAG
AGGACCTGCTGCGGAAGCAGCGGACCTTCGACAACGGCAGCATCCCCCACCAGA
TCCACCTGGGAGAGCTGCACGCCATTCTGCGGCGGCAGGAAGATTTTTACCCATT
CCTGAAGGACAACCGGGAAAAGATCGAGAAGATCCTGACCTTCCGCATCCCCTA
CTACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCGC
TTCCGCCCAGAGCTTCATCGAGCGGATGACCAACTTCGATAAGAACCTGCCCAAC
GAGAAGGTGCTGCCCAAGCACAGCCTGCTGTACGAGTACTTCACCGTGTATAAC
GAGCTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGGAAA
GTGACCGTGAAGCAGCTGAAAGAGGACTACTTCAAGAAAATCGAGTGCTTCGAC
TCCGTGGAAATCTCCGGCGTGGAAGATCGGTTCAACGCCTCCCTGGGCACATACC
ACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGA
TGATCGAGGAACGGCTGAAAACCTATGCCCACCTGTTCGACGACAAAGTGATGA
AGCAGCTGAAGCGGCGGAGATACACCGGCTGGGGCAGGCTGAGCCGGAAGCTG
ATCAACGGCATCCGGGACAAGCAGTCCGGCAAGACAATCCTGGATTTCCTGAAG
TCCGACGGCTTCGCCAACAGAAACTTCATGCAGCTGATCCACGACGACAGCCTG
ACCTTTAAAGAGGACATCCAGAAAGCCCAGGTGTCCGGCCAGGGCGATAGCCTG
CACGAGCACATTGCCAATCTGGCCGGCAGCCCCGCCATTAAGAAGGGCATCCTG
CAGACAGTGAAGGTGGTGGACGAGCTCGTGAAAGTGATGGGCCGGCACAAGCCC
GAGAACATCGTGATCGAAATGGCCAGAGAGAACCAGACCACCCAGAAGGGACA
GAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAG
AAGCTGTACCTGTACTACCTGCAGAATGGGCGGGATATGTACGTGGACCAGGAA
CTGGACATCAACCGGCTGTCCGACTACGATGTGGACGCTATCGTGCCTCAGAGCT
TTCTGAAGGACGACTCCATCGACAACAAGGTGCTGACCAGAAGCGACAAGAACC
GGGGCAAGAGCGACAACGTGCCCTCCGAAGAGGTCGTGAAGAAGATGAAGAAC
TACTGGCGGCAGCTGCTGAACGCCAAGCTGATTACCCAGAGAAAGTTCGACAAT
CTGACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCCTG
GACTCCCGGATGAACACTAAGTACGACGAGAATGACAAGCTGATCCGGGAAGTG
AAAGTGATCACCCTGAAGTCCAAGCTGGTGTCCGATTTCCGGAAGGATTTCCAGT
TTTACAAAGTGCGCGAGATCAACAACTACCACCACGCCCACGACGCCTACCTGA
ACGCCGTCGTGGGAACCGCCCTGATCAAAAAGTACCCTAAGCTGGAAAGCGAGT
TCGTGTACGGCGACTACAAGGTGTACGACGTGCGGAAGATGATCGCCAAGAGCG
AGCAGGAAATCGGCAAGGCTACCGCCAAGTACTTCTTCTACAGCAACATCATGA
ACTTTTTCAAGACCGAGATTACCCTGGCCAACGGCGAGATCCGGAAGCGGCCTCT
GATCGAGACAAACGGCGAAACCGGGGAGATCGTGTGGGATAAGGGCCGGGATT
TTGCCACCGTGCGGAAAGTGCTGAGCATGCCCCAAGTGAATATCGTGAAAAAGA
CCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGG
GCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACA
AAGAAGTGAAAAAGGACCTGATCATCAAGCTGCCTAAGTACTCCCTGTTCGAGC
TGGAAAACGGCCGGAAGAGAATGCTGGCCTCTGCCGGCGAACTGCAGAAGGGA
AACGAACTGGCCCTGCCCTCCAAATATGTGAACTTCCTGTACCTGGCCAGCCACT
ATGAGAAGCTGAAGGGCTCCCCCGAGGATAATGAGCAGAAACAGCTGTTTGTGG
AACAGCACAAGCACTACCTGGACGAGATCATCGAGCAGATCAGCGAGTTCTCCA
AGAGAGTGATCCTGGCCGACGCTAATCTGGACAAAGTGCTGTCCGCCTACAACA
AGCACCGGGATAAGCCCATCAGAGAGCAGGCCGAGAATATCATCCACCTGTTTA
CCCTGACCAATCTGGGAGCCCCTGCCGCCTTCAAGTACTTTGACACCACCATCGA
CCGGAAGAGGTACACCAGCACCAAAGAGGTGCTGGACGCCACCCTGATCCACCA
GAGCATCACCGGCCTGTACGAGACACGGATCGACCTGTCTCAGCTGGGAGGTGA
CTCCGGCGGAAGCTCTGGTGGCAGCAAGCGGACCGCCGACGGCTCTGAATTCGA
GAGCCCTAAGAAGAAAAGAAAGGTGAGCGGAGGCTCTAGCGGCGGAAGCACCC
TGAACATTGAAGACGAGTATAGACTGCATGAAACAAGCAAGGAACCCGACGTGT
CCCTGGGCTCCACCTGGCTGTCCGACTTTCCCCAGGCCTGGGCCGAGACAGGAGG
AATGGGCCTGGCCGTGCGGCAGGCACCCCTGATCATCCCTCTGAAGGCCACCTCT
ACACCCGTGAGCATCAAGCAGTACCCTATGTCTCAGGAGGCCAGACTGGGCATC
AAGCCTCACATCCAGAGGCTGCTGGACCAGGGCATCCTGGTGCCATGCCAGAGC
CCCTGGAACACACCACTGCTGCCCGTGAAGAAGCCAGGCACCAATGACTATAGA
CCCGTGCAGGATCTGAGAGAGGTGAACAAGAGGGTGGAGGATATCCACCCCACC
GTGCCCAACCCTTACAATCTGCTGTCCGGCCTGCCCCCTTCTCACCAGTGGTATA
CAGTGCTGGACCTGAAGGATGCCTTCTTTTGTCTGAGACTGCACCCTACCAGCCA
GCCACTGTTCGCCTTTGAGTGGAGGGACCCTGAGATGGGCATCTCTGGCCAGCTG
ACCTGGACACGCCTGCCTCAGGGCTTCAAGAATAGCCCAACACTGTTTAACGAG
GCCCTGCACCGCGACCTGGCAGATTTCCGGATCCAGCACCCAGATCTGATCCTGC
TGCAGTACGTGGACGATCTGCTGCTGGCCGCCACCAGCGAGCTGGATTGCCAGC
AGGGAACACGCGCCCTGCTGCAGACCCTGGGAAACCTGGGATATAGGGCATCCG
CCAAGAAGGCCCAGATCTGTCAGAAGCAGGTGAAGTACCTGGGCTATCTGCTGA
AGGAGGGCCAGAGATGGCTGACAGAGGCCAGGAAGGAGACAGTGATGGGCCAG
CCAACACCCAAGACCCCAAGACAGCTGAGGGAGTTCCTGGGCAAAGCAGGATTT
TGCAGGCTGTTCATCCCAGGATTCGCAGAGATGGCAGCACCTCTGTACCCACTGA
CCAAGCCGGGCACCCTGTTTAATTGGGGCCCTGACCAGCAGAAGGCCTATCAGG
AGATCAAGCAGGCCCTGCTGACAGCACCAGCCCTGGGCCTGCCAGACCTGACCA
AGCCTTTCGAGCTGTTTGTGGATGAGAAGCAGGGCTACGCCAAGGGCGTGCTGA
CCCAGAAGCTGGGACCATGGAGACGGCCCGTGGCCTATCTGTCCAAGAAGCTGG
ACCCAGTGGCAGCAGGATGGCCACCATGCCTGAGGATGGTGGCAGCAATCGCCG
TGCTGACAAAGGATGCCGGCAAGCTGACCATGGGACAGCCACTGGTCATCCTGG
CACCACACGCAGTGGAGGCCCTGGTGAAGCAGCCTCCAGATCGCTGGCTGTCTA
ACGCCCGGATGACACACTACCAGGCCCTGCTGCTGGACACCGATCGCGTGCAGT
TTGGCCCTGTGGTGGCCCTGAATCCAGCCACCCTGCTGCCTCTGCCAGAGGAGGG
CCTGCAGCACAACTGTCTGGACATCCTGGCAGAGGCACACGGAACAAGGCCAGA
CCTGACCGATCAGCCCCTGCCTGACGCCGATCACACATGGTATACCGATGGAAG
CTCCCTGCTGCAGGAGGGCCAGAGGAAGGCAGGAGCAGCAGTGACCACAGAGA
CAGAAGTGATCTGGGCCAAGGCCCTGCCAGCAGGCACATCCGCCCAGCGGGCCG
AGCTGATCGCCCTGACCCAGGCCCTGAAGATGGCCGAGGGCAAGAAGCTGAACG
TGTACACAGACTCCAGATATGCCTTCGCCACCGCACACATCCACGGAGAGATCTA
CAGGCGCCGGGGCTGGCTGACCTCTGAGGGCAAGGAGATCAAGAACAAGGATG
AGATCCTGGCCCTGCTGAAGGCCCTGTTTCTGCCCAAGCGGCTGAGCATCATCCA
CTGTCCTGGACACCAGAAGGGACACTCCGCCGAGGCAAGGGGCAATCGGATGGC
CGACCAGGCCGCCAGAAAGGCTGCTATTACTGAAACTCCCGACACTTCCACTCTG
CTGATTGAAAACTCCTCCCCTTCTGGCGGCTCAAAAAGAACCGCCGACGGCAGC
GAATTCGAGTCTCCCAAGAAGAAGAGGAAAGTCGGCTCTGGCCCTGCCGCTAAG
AGAGTGAAGCTGGACTAA

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for enhancing prime editing in a hematopoietic stem and progenitor cell (HSPC), the method comprising contacting the HSPC with the following components under conditions permissive for genome editing, the components comprising
a programmable DNA binding protein;
a DNA nickase;
a polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide or a primer binding sequence that binds the nicked single stranded DNA and a template sequence to specify the edit to be introduced by the polymerase; and
a VPX polypeptide or a polynucleotide encoding a VPX polypeptide, and/or exogenous deoxynucleosides or deoxynucleotides.

2. The method of claim 1, wherein the programmable DNA binding protein comprises a Cas9 having nickase activity.

3. A method of modifying an HSPC genome, the method comprising contacting the HSPC with the following components under conditions permissive for genome editing, the components comprising
a programmable DNA binding protein;
a DNA nickase;
a polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide; and
a VPX polypeptide or a polynucleotide encoding a VPX polypeptide and/or exogenous deoxynucleosides or deoxynucleotides.

4. The method of claim 3, wherein the programmable DNA binding protein comprises a Cas9 having nickase activity.

5. The method of claim 1, wherein the polymerase is a reverse transcriptase that comprises a mutation that increases dNTPs affinity.

6. The method of claim 5, wherein the mutation is V223M.

7. The method of any of claim 1, wherein the polymerase is RNA-dependent RNA polymerase nsp12.

8. An HSPC comprising:
a programmable DNA binding protein;
a DNA nickase;
a polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide; and
a VPX polypeptide or a polynucleotide encoding a VPX polypeptide.

9. A method for enhancing prime editing in a hematopoietic stem and progenitor cell (HSPC), the method comprising contacting the HSPC with the following components under conditions permissive for genome editing, the components comprising
a programmable DNA binding protein;
a DNA nickase;
a DNA polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide; and
a VPX polypeptide or a polynucleotide encoding a VPX polypeptide,
wherein the template is designed to evade mismatch repair.

10. A method for enhancing prime editing in a hematopoietic stem and progenitor cell (HSPC), the method comprising contacting the HSPC with the following components under conditions permissive for genome editing, the components comprising
a programmable DNA binding protein;
a DNA nickase;
a DNA polymerase;
a prime editing guide RNA comprising a gRNA target sequence that binds target DNA, a primer binding sequence that primes DNA synthesis, and a template containing an edited RNA sequence, wherein the guide RNA is capable of directing the fusion protein to a target sequence in a polynucleotide;
a VPX polypeptide or a polynucleotide encoding a VPX polypeptide; and
a mismatch repair inhibitor.

11. The method of claim 10, wherein the mismatch repair inhibitor is a dominant negative human MutL homolog (MLH1) peptide.

12. A method for enhancing prime editing in a human hematopoietic stem and progenitor cell (HSPC), the method comprising contacting the HSPC with the following components under conditions permissive for genome editing, the components comprising
a programmable DNA binding protein;
a DNA nickase;
a reverse transcriptase;
a pair of prime editing guide RNAs each comprising a gRNA target sequence that binds target DNA, a primer binding sequence that primes DNA synthesis, and a template containing an edited RNA sequence, wherein the guide RNA is capable of directing the fusion protein to a target sequence in a polynucleotide;
a VPX polypeptide or a polynucleotide encoding a VPX polypeptide,
wherein the template in each prime editing guide RNA is complementary to the template in the other prime editing guide RNA.

13. The method of claim 12, wherein the pair of prime editing guide RNAs is designed to prevent mismatches when a genome of the HSPC is edited by the components.

14. The method of claim 12, wherein the polymerase is a DNA-dependent or an RNA-dependent DNA polymerase.

15. The method of claim 12, wherein the RNA-dependent DNA polymerase is reverse transcriptase.

16. The method of claim 15, wherein the reverse transcriptase comprises a mutation that increases dNTPs affinity.

17. The method of claim 16, wherein the mutation is V223M.

18. The method of claim 12, wherein the polymerase is an RNA-dependent RNA polymerase.

19. The method of claim 18, wherein the RNA-dependent RNA polymerase is nsp12.

20. The method of any one of claim 1, wherein the template is designed to evade mismatch repair.
